(19) United States
(12) Patent Application Publication
(10) Pub. No.: US 2023/0381335 A1
(43) Pub. Date: Nov. 30, 2023

(54) LUNG CANCER COMBINATION THERAPY WITH IL-2 CONJUGATES AND AN ANTI-PD-1 ANTIBODY OR ANTIGEN-BINDING FRAGMENT THEREOF

(71) Applicants: SYNTHORX, INC., La Jolla, CA (US); MSD INTERNATIONAL GMBH, Luzern (CH)

(72) Inventors: Giovanni Abbadessa, Winchester, MA (US); Carolina E. Caffaro, San Diego, CA (US); Brigitte Demers, Paris (FR); Joseph Leveque, San Diego, CA (US); Wan-Ju Meng, Cambridge, MA (US); Jerod Ptacin, San Diego, CA (US)

(21) Appl. No.: 18/447,808

(22) Filed: Aug. 10, 2023

Related U.S. Application Data
(63) Continuation of application No. PCT/US2022/016217, filed on Feb. 11, 2022.
(60) Provisional application No. 63/149,078, filed on Feb. 12, 2021.
(60) Provisional application No. 63/253,903, filed on Oct. 8, 2021.
(60) Provisional application No. 63/276,954, filed on Nov. 8, 2021.

(51) Int. Cl. A61K 47/68; C07K 16/28; C07K 16/24; A61K 31/282; A61K 33/243; A61P 35/00; A61K 31/337; A61K 31/167; A61K 31/138

(52) U.S. Cl. CPC A61K 47/6845; C07K 16/2818; C07K 16/246; A61K 31/282; A61K 33/243; A61P 35/00; A61K 31/337; A61K 31/167; A61K 31/138

(57) ABSTRACT

Disclosed herein are methods for treating a lung cancer in a subject in need thereof, comprising administering IL-2 conjugates in combination with the anti-PD-1 antibody or antigen-binding fragment thereof (e.g., pembrolizumab).

### CROSS-REFERENCE TO RELATED APPLICATIONS

This application is a continuation of International Patent Application No.: PCT/US2022/016217, filed Feb. 11, 2022, which claims the benefit of priority to U.S. Provisional Application No. 63/149,078, filed on Feb. 12, 2021, U.S. Provisional Application No. 63/253,903, filed on Oct. 8, 2021, and U.S. Provisional Application No. 63/276,954, filed on Nov. 8, 2021, the disclosure of each of which is hereby incorporated by reference in its entirety.

### REFERENCE TO A SEQUENCE LISTING

This application incorporates by reference the Sequence Listing submitted in Computer Readable Form as file SeqList_052838-00136.xml, created on Aug. 9, 2023 and is 22,018 bytes in size.

### BACKGROUND OF THE DISCLOSURE

Distinct populations of T cells modulate the immune system to maintain immune homeostasis and tolerance. For example, regulatory T (Treg) cells prevent inappropriate responses by the immune system by preventing pathological self-reactivity while cytotoxic T cells target and destroy infected cells and/or cancerous cells. In some instances, modulation of the different populations of T cells provides an option for treatment of a disease or indication.

Cytokines comprise a family of cell signaling proteins such as chemokines, interferons, interleukins, lymphokines, tumor necrosis factors, and other growth factors playing roles in innate and adaptive immune cell homeostasis. Cytokines are produced by immune cells such as macrophages, B lymphocytes, T lymphocytes and mast cells, endothelial cells, fibroblasts, and different stromal cells. In some instances, cytokines modulate the balance between humoral and cell-based immune responses.

Interleukins are signaling proteins that modulate the development and differentiation of T and B lymphocytes, cells of the monocytic lineage, neutrophils, basophils, eosinophils, megakaryocytes, and hematopoietic cells. Interleukins are produced by helper CD4+ T and B lymphocytes, monocytes, macrophages, endothelial cells, and other tissue residents.

In some instances, interleukin 2 (IL-2) signaling is used to modulate T cell responses and subsequently for treatment of a cancer.

PD-1 is recognized as an important player in immune regulation and the maintenance of peripheral tolerance. PD-1 is moderately expressed on naive T, B and NKT cells and up-regulated by T/B cell receptor signaling on lymphocytes, monocytes and myeloid cells (Sharpe et al., The function of programmed cell death 1 and its ligands in regulating autoimmunity and infection. Nature Immunology (2007); 8:239-245).

Two known ligands for PD-1, PD-L1 (B7-H1) and PD-L2 (B7-DC), are expressed in human cancers arising in various tissues. In large sample sets of e.g. ovarian, renal, colorectal, pancreatic, liver cancers and melanoma, it was shown that PD-L1 expression correlated with poor prognosis and reduced overall survival irrespective of subsequent treatment (Dong et al., Nat Med. 8(8):793-800 (2002); Yang et al. Invest Ophthalmol Vis Sci. 49: 2518-2525 (2008); Ghebeh et al. Neoplasia 8:190-198 (2006); Hamanishi et al., Proc. Natl. Acad. Sci. USA 104: 3360-3365 (2007); Thompson et al., Cancer 5: 206-211 (2006); Nomi et al., Clin. Cancer Research 13:2151-2157 (2007); Ohigashi et al., Clin. Cancer Research 11: 2947-2953 (2005); Inman et al., Cancer 109: 1499-1505 (2007); Shimauchi et al. Int. J. Cancer 121:2585-2590 (2007); Gao et al. Clin. Cancer Research 15: 971-979 (2009); Nakanishi J Cancer Immunol Immunother. 56: 1173-1182 (2007); and Hino et al., Cancer 00: 1-9 (2010)).

Similarly, PD-1 expression on tumor infiltrating lymphocytes was found to mark dysfunctional T cells in breast cancer and melanoma (Ghebeh et al, BMC Cancer. 2008 8:5714-15 (2008); Ahmadzadeh et al., Blood 114: 1537-1544 (2009)) and to correlate with poor prognosis in renal cancer (Thompson et al., Clinical Cancer Research 15: 1757-1761(2007)). Thus, it has been proposed that PD-L1 expressing tumor cells interact with PD-1 expressing T cells to attenuate T cell activation and evasion of immune surveillance, thereby contributing to an impaired immune response against the tumor.

Immune checkpoint therapies targeting the PD-1 axis have resulted in groundbreaking improvements in clinical response in multiple human cancers (Brahmer et al., N Engl J Med 2012, 366: 2455-65; Garon et al. N Engl J Med 2015, 372: 2018-28; Hamid et al., N Engl J Med 2013, 369: 134-44; Robert et al., Lancet 2014, 384: 1109-17; Robert et al., N Engl J Med 2015, 372: 2521-32; Robert et al., N Engl J Med 2015, 372: 320-30; Topalian et al., N Engl J Med 2012, 366: 2443-54; Topalian et al., J Clin Oncol 2014, 32: 1020-30; Wolchok et al., N Engl J Med 2013, 369: 122-33). Immune therapies targeting the PD-1 axis include monoclonal antibodies directed to the PD-1 receptor (KEYTRUDA (pembrolizumab), Merck and Co., Inc., Kenilworth, NJ, USA and OPDIVO (nivolumab), Bristol-Myers Squibb Company, Princeton, NJ, USA) and also those that bind to the PD-L1 ligand (MPDL3280A; TECENTRIQ™ (atezolizumab), Genentech, San Francisco, CA, USA; IMFINZI (durvalumab), AstraZeneca Pharmaceuticals LP, Wilmington, DE; BAVENCIO (avelumab), Merck KGaA, Darmstadt, Germany). Both therapeutic approaches have demonstrated anti-tumor effects in numerous cancer types.

Accordingly, in one aspect, provided herein are methods of treating lung cancer in a subject comprising administering an IL-2 conjugate in combination with an anti-PD-1 antibody or antigen-binding fragment thereof, e.g., the anti-PD-1 antibody pembrolizumab.

### SUMMARY OF THE DISCLOSURE

Described herein are methods of treating cancer in a subject in need thereof, comprising administering to a subject (a) about 8 μg/kg, 16 μg/kg, 24 μg/kg, or 32 μg/kg of an IL-2 conjugate, and (b) an anti-PD-1 antibody or antigen-binding fragment thereof, wherein the IL-2 conjugate comprises the amino acid sequence of SEQ ID NO: 1 having an unnatural amino acid residue described herein at position 64, e.g., the amino acid sequence of SEQ ID NO: 2, wherein the anti-PD-1 antibody or antigen-binding fragment thereof comprises light chain complementarity determining regions (CDRs) comprising a sequence of amino acids as set forth in SEQ ID NOs: 11, 12 and 13 and heavy chain CDRs comprising a sequence of amino acids as set forth in SEQ ID NOs: 16, 17 and 18.

In some embodiments, administration of the IL-2 conjugate and the anti-PD-1 antibody or antigen-binding fragment thereof provides improved results in treatment of lung cancer or a subtype thereof relative to existing therapies. For example, improved results may be in terms of the frequency of favorable outcomes, such as complete responses, elimination of target lesions, reduction of the size of target lesions, partial responses, stable disease, or slowing the growth of target lesions. In some embodiments, administration of the IL-2 conjugate and the anti-PD-1 antibody or antigen-binding fragment thereof provides improved safety relative to existing lung cancer or IL-2 therapies, or to monotherapy using an IL-2 conjugate or the anti-PD-1 antibody or antigen-binding fragment thereof alone. For example, improved safety may be in terms of avoidance or reduced frequency of adverse events, such as Grade 4 adverse events; vascular leak syndrome (e.g., Grade 2, Grade 3, and/or Grade 4 vascular leak syndrome); capillary leak syndrome; extravasation of plasma proteins and fluid into the extravascular space in the subject; hypotension and/or reduced organ perfusion in the subject; impaired neutrophil function in the subject; a drop in mean arterial blood pressure in the subject following administration; a systolic blood pressure below 90 mm Hg or a 20 mm Hg drop from baseline systolic pressure; eosinophilia; edema or impairment of kidney or liver function; or reduced chemotaxis in the subject. In some embodiments, improved safety may be in terms of absence of increased risk of disseminated infection in the subject; absence of exacerbation of a pre-existing or initial presentation of an autoimmune disease or an inflammatory disorder in the subject. In some embodiments, administration of the IL-2 conjugate and the anti-PD-1 antibody or antigen-binding fragment thereof provides improved results in terms of a combination of one or more of the favorable outcomes discussed above or disclosed elsewhere herein or frequencies thereof and one or more of the improvements in safety discussed above or disclosed elsewhere herein.

Exemplary embodiments include the following.

Embodiment 1 is a method of treating lung cancer in a subject in need thereof, comprising administering to the subject (a) an IL-2 conjugate, and (b) an anti-PD-1 antibody or antigen-binding fragment thereof, wherein:
the IL-2 conjugate comprises the amino acid sequence of SEQ ID NO: 1 wherein the amino acid at position P64 is replaced by the structure of Formula (I):

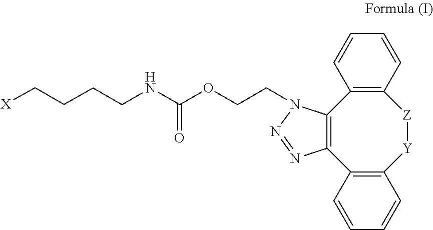

wherein:
Z is CH₂ and Y is

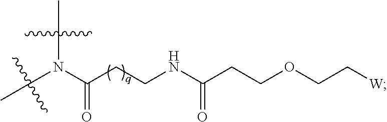

Y is CH₂ and Z is

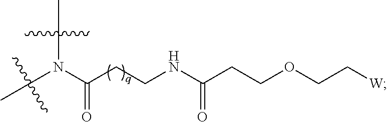

Z is CH₂ and Y is

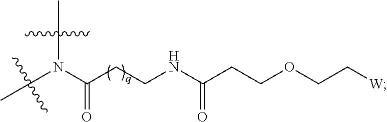

or
Y is CH₂ and Z is

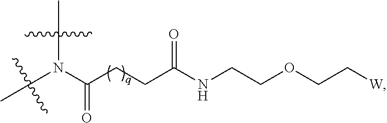

W is a PEG group having an average molecular weight of about 25 kDa-35 kDa;
q is 1, 2, or 3;
X is an L-amino acid having the structure:

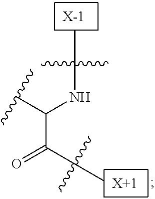 [SMILES: "X-1" ; *NC(*)C(*)=O ; "X+1"]

X−1 indicates the point of attachment to the preceding amino acid residue; and
X+1 indicates the point of attachment to the following amino acid residue; and
wherein the anti-PD-1 antibody or antigen-binding fragment thereof comprises light chain complementarity determining regions (CDRs) comprising a sequence of amino acids as set forth in SEQ ID NOs: 11, 12 and 13 and heavy chain CDRs comprising a sequence of amino acids as set forth in SEQ ID NOs: 16, 17 and 18.

Embodiment 2 is a method of treating lung cancer in a subject in need thereof, comprising administering to the subject (a) an IL-2 conjugate, and (b) an anti-PD-1 antibody or antigen-binding fragment thereof, wherein:
the lung cancer is non-squamous non-small cell lung cancer (NSCLC), pleural mesothelioma, unresectable lung cancer, stage IV lung cancer, NSCLC having a PD-L1 tumor proportion score greater than or equal to 50%, or NSCLC having a PD-L1 tumor progession score of less than 50% or of 1-49%; and
the IL-2 conjugate comprises the amino acid sequence of SEQ ID NO: 1 wherein the amino acid at position P64 is replaced by the structure of Formula (I):

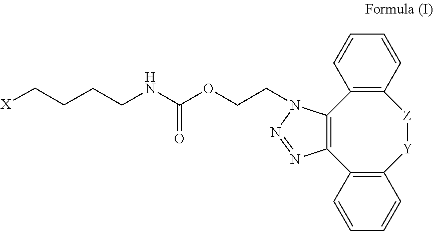

wherein:
Z is CH₂ and Y is

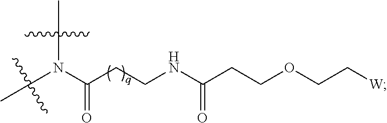

Y is CH₂ and Z is

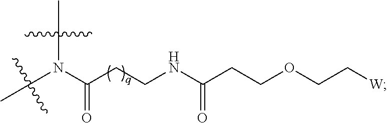

Z is CH₂ and Y is

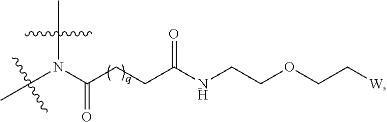

or
Y is CH₂ and Z is

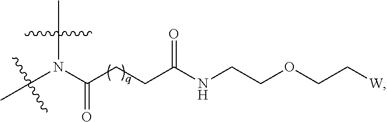

W is a PEG group having an average molecular weight of about 25 kDa-35 kDa;
q is 1, 2, or 3;
X is an L-amino acid having the structure:

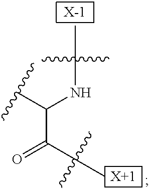

X−1 indicates the point of attachment to the preceding amino acid residue; and
X+1 indicates the point of attachment to the following amino acid residue,
wherein the anti-PD-1 antibody or antigen-binding fragment thereof comprises light chain complementarity determining regions (CDRs) comprising a sequence of amino acids as set forth in SEQ ID NOs: 11, 12 and 13 and heavy chain CDRs comprising a sequence of amino acids as set forth in SEQ ID NOs: 16, 17 and 18.

Embodiment 3 is a method of treating lung cancer in a subject in need thereof, comprising:
selecting a subject having lung cancer, wherein the subject is selected on the basis of one or more attributes comprising (i) the lung cancer being non-squamous non-small cell lung cancer (NSCLC); (ii) the lung cancer being pleural mesothelioma; (iii) the lung cancer being unresectable lung cancer; (iv) the lung cancer being stage IV lung cancer; (v) the lung cancer being NSCLC having a PD-L1 tumor proportion score greater than or equal to 50%; (vi) the lung cancer being NSCLC having a PD-L1 tumor progession score of less than 50% or of 1-49%; and
administering to the subject (a) an IL-2 conjugate, and (b) an anti-PD-1 antibody or antigen-binding fragment thereof, wherein:
the IL-2 conjugate comprises the amino acid sequence of SEQ ID NO: 1 wherein the amino acid at position P64 is replaced by the structure of Formula (I):

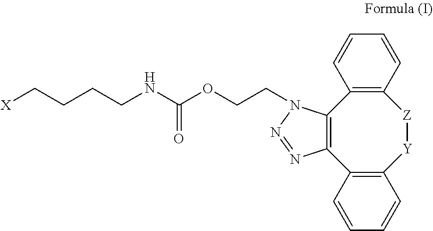

wherein:
Z is CH₂ and Y is

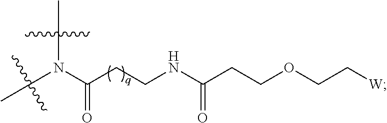

Y is CH₂ and Z is

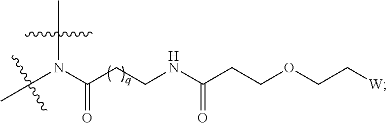

Z is CH₂ and Y is

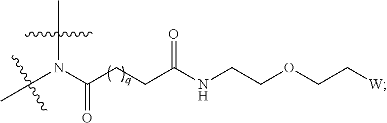

or
Y is CH₂ and Z is

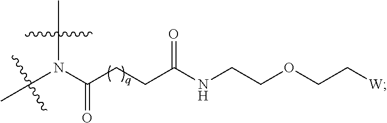

W is a PEG group having an average molecular weight of about 25 kDa-35 kDa;
q is 1, 2, or 3;
X is an L-amino acid having the structure:

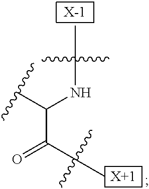

X−1 indicates the point of attachment to the preceding amino acid residue; and
X+1 indicates the point of attachment to the following amino acid residue,
wherein the anti-PD-1 antibody or antigen-binding fragment thereof comprises light chain complementarity determining regions (CDRs) comprising a sequence of amino acids as set forth in SEQ ID NOs: 11, 12 and 13 and heavy chain CDRs comprising a sequence of amino acids as set forth in SEQ ID NOs: 16, 17 and 18.

Embodiment 4 is the method of any one of embodiments 1-3, further comprising administering cisplatin to the subject.

Embodiment 5 is a method of treating lung cancer in a subject in need thereof, comprising administering to the subject (a) an IL-2 conjugate, (b) an anti-PD-1 antibody or antigen-binding fragment thereof, and (c) cisplatin, wherein:
the IL-2 conjugate comprises the amino acid sequence of SEQ ID NO: 1 wherein the amino acid at position P64 is replaced by the structure of Formula (I):

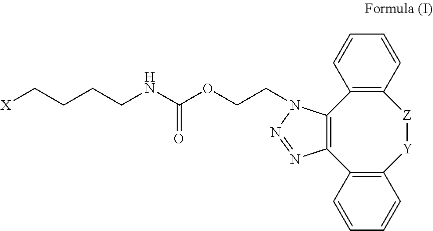

wherein:
Z is CH₂ and Y is

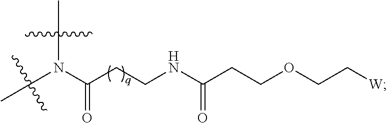

Y is CH₂ and Z is

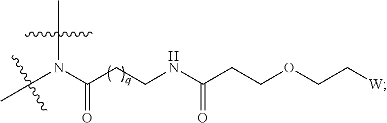

Z is CH₂ and Y is

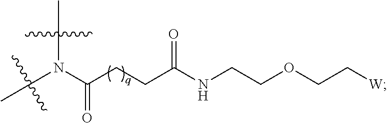

or
Y is CH₂ and Z is

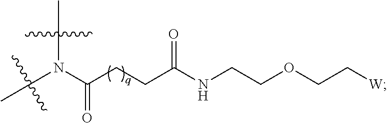

W is a PEG group having an average molecular weight of about 25 kDa-35 kDa;
q is 1, 2, or 3;
X is an L-amino acid having the structure:

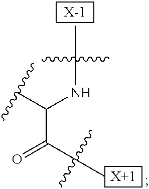

X−1 indicates the point of attachment to the preceding amino acid residue; and
X+1 indicates the point of attachment to the following amino acid residue, wherein the anti-PD-1 antibody or antigen-binding fragment thereof comprises light chain complementarity determining regions (CDRs) comprising a sequence of amino acids as set forth in SEQ ID NOs: 11, 12 and 13 and heavy chain CDRs comprising a sequence of amino acids as set forth in SEQ ID NOs: 16, 17 and 18.

Embodiment 6 is the method of any one of embodiments 1-5, wherein the lung cancer is NSCLC.

Embodiment 7 is the method of any one of embodiments 1-6, wherein the lung cancer is unresectable.

Embodiment 8 is the method of any one of embodiments 1-7, wherein the lung cancer is stage IV.

Embodiment 9 is the method of any one of embodiments 1-8, wherein the lung cancer is non-squamous NSCLC.

Embodiment 10 is the method of any one of embodiments 1-9, wherein the lung cancer is pleural mesothelioma.

Embodiment 11 is the method of any one of embodiments 1-10, comprising administering to the subject about 8 μg/kg of the IL-2 conjugate.

Embodiment 12 is the method of any one of embodiments 1-10, comprising administering to the subject about 16 μg/kg of the IL-2 conjugate.

Embodiment 13 is the method of any one of embodiments 1-10, comprising administering to the subject about 24 μg/kg of the IL-2 conjugate.

Embodiment 14 is the method of any one of embodiments 1-10, comprising administering to the subject about 32 μg/kg of the IL-2 conjugate.

Embodiment 15 is the method of any one of embodiments 1-14, further comprising administering pemetrexed to the subject.

Embodiment 16 is the method of any one of embodiments 1-15, further comprising administering carboplatin to the subject.

Embodiment 17 is the method of any one of embodiments 1-16, further comprising administering nab-paclitaxel to the subject.

Embodiment 18 is the method of any one of embodiments 1-17, wherein in the IL-2 conjugate the PEG group has an average molecular weight of about 30 kDa.

Embodiment 19 is the method of any one of embodiments 1-18, wherein in the IL-2 conjugate Z is CH₂ and Y is

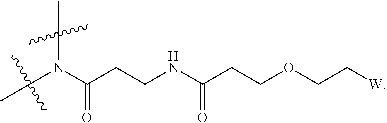

Embodiment 20 is the method of any one of embodiments 1-18, wherein in the IL-2 conjugate Y is CH₂ and Z is

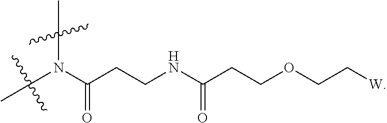

Embodiment 21 is the method of any one of embodiments 1-18, wherein in the IL-2 conjugate Z is CH₂ and Y is

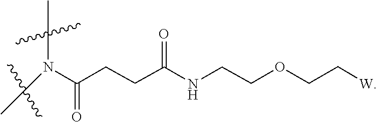

Embodiment 22 is the method of any one of embodiments 1-18, wherein in the IL-2 conjugate Y is CH₂ and Z is

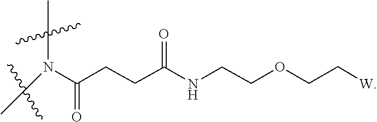

Embodiment 23 is the method of any one of embodiments 1-18, wherein the structure of Formula (I) has the structure of Formula (IV) or Formula (V), or is a mixture of Formula (IV) and Formula (V):

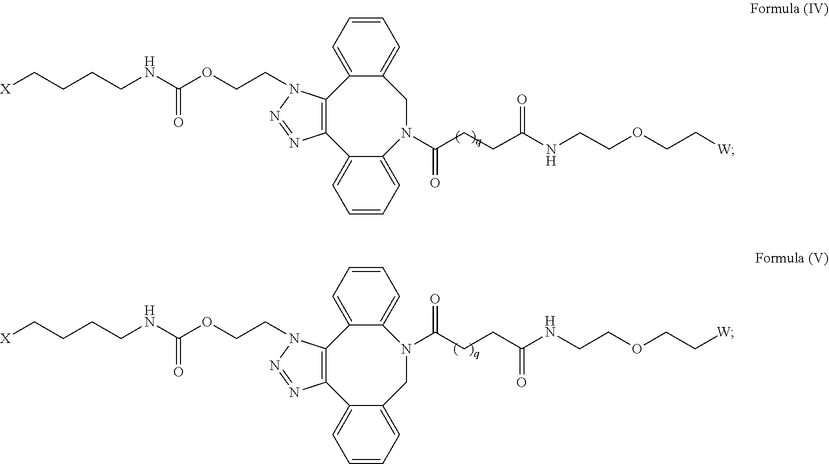

wherein:
q is 1, 2, or 3;
X is an L-amino acid having the structure:

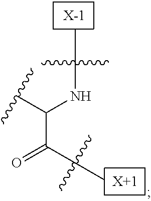

X−1 indicates the point of attachment to the preceding amino acid residue; and
X+1 indicates the point of attachment to the following amino acid residue.

Embodiment 24 is the method of any one of embodiments 1-18, wherein the structure of Formula (I) has the structure of Formula (XII) or Formula (XIII), or is a mixture of Formula (XII) and Formula (XIII):

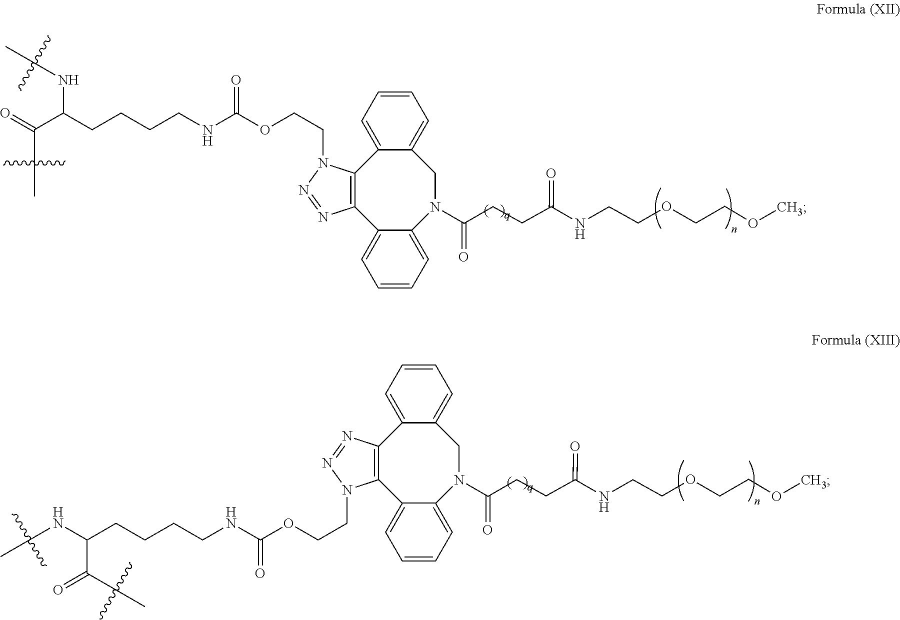

wherein:
n is an integer such that —(OCH₂CH₂)ₙ—OCH₃ has a molecular weight of about 30 kDa;
q is 1, 2, or 3; and
the wavy lines indicate covalent bonds to amino acid residues within SEQ ID NO: 1 that are not replaced.

Embodiment 25 is the method of any one of embodiments 1-24, wherein q is 1.

Embodiment 26 is the method of any one of embodiments 1-24, wherein q is 2.

Embodiment 27 is the method of any one of embodiments 1-24, wherein q is 3.

Embodiment 28 is the method of any one of embodiments 1-27, wherein the IL-2 conjugate is administered to the subject about once every two weeks, about once every three weeks, or about once every 4 weeks.

Embodiment 29 is the method of any one of embodiments 1-28, wherein the IL-2 conjugate and the anti-PD-1 antibody or antigen-binding fragment thereof are administered to the subject about once every two weeks, about once every three weeks, or about once every 4 weeks.

Embodiment 30 is the method of any one of embodiments 1-29, wherein the IL-2 conjugate is a pharmaceutically acceptable salt, solvate, or hydrate.

Embodiment 31 is the method of any one of embodiments 1-30, wherein the method comprises administering:
(i) about 200 mg of an anti-PD-1 antibody, or antigen binding fragment thereof to the patient every approximately three weeks; or
(ii) about 400 mg of an anti-PD-1 antibody, or antigen binding fragment thereof, to the patient every approximately six weeks.

Embodiment 32 is the method of any one of embodiments 1-31, wherein the IL-2 conjugate and the anti-PD-1 antibody or antigen-binding fragment thereof are administered separately.

Embodiment 33 is the method of embodiment 32, wherein the IL-2 conjugate and the anti-PD-1 antibody or antigen-binding fragment thereof are administered sequentially.

Embodiment 34 is the method of embodiment 32 or 33, wherein the IL-2 conjugate is administered before the anti-PD-1 antibody or antigen-binding fragment thereof.

Embodiment 35 is the method of embodiment 32 or 33, wherein the IL-2 conjugate is administered after the anti-PD-1 antibody or antigen-binding fragment thereof.

Embodiment 36 is the method of any one of embodiments 1-35, wherein the IL-2 conjugate is administered to the subject by subcutaneous administration.

Embodiment 37 is the method of any one of embodiments 1-36, wherein the IL-2 conjugate and the anti-PD-1 antibody or antigen-binding fragment thereof are administered to the subject by subcutaneous administration.

Embodiment 38 is the method of any one of embodiments 1-35, wherein the IL-2 conjugate is administered to the subject by intravenous administration.

Embodiment 39 is the method of any one of embodiments 1-35, wherein the IL-2 conjugate and the anti-PD-1 antibody or antigen-binding fragment thereof are administered to the subject by intravenous administration.

Embodiment 40 is the method of any one of embodiments 1-39, further comprising administering acetaminophen to the subject.

Embodiment 41 is the method of any one of embodiments 1-40, further comprising administering diphenhydramine to the subject.

Embodiment 42 is the method of embodiment 40 or 41, wherein the acetaminophen and/or diphenhydramine is administered to the subject before administering the IL-2 conjugate.

Embodiment 43 is the method of any one of embodiments 1-42, further comprising selecting the subject to whom the IL-2 conjugate and the anti-PD-1 antibody or antigen-binding fragment thereof are administered at least in part on the basis of the lung cancer being non-squamous NSCLC.

Embodiment 44 is the method of any one of embodiments 1-43, further comprising selecting the subject to whom the IL-2 conjugate and the anti-PD-1 antibody or antigen-binding fragment thereof are administered at least in part on the basis of the lung cancer being pleural mesothelioma.

Embodiment 45 is the method of any one of embodiments 1-44, further comprising selecting the subject to whom the IL-2 conjugate and the anti-PD-1 antibody or antigen-binding fragment thereof are administered at least in part on the basis of the lung cancer being unresectable lung cancer.

Embodiment 46 is the method of any one of embodiments 1-45, further comprising selecting the subject to whom the IL-2 conjugate and the anti-PD-1 antibody or antigen-binding fragment thereof are administered at least in part on the basis of the lung cancer being stage IV lung cancer.

Embodiment 47 is the method of any one of embodiments 1-46, further comprising selecting the subject to whom the IL-2 conjugate and the anti-PD-1 antibody or antigen-binding fragment thereof are administered at least in part on the basis of the lung cancer being NSCLC having a PD-L1 tumor proportion score greater than or equal to 50%.

Embodiment 48 is the method of any one of embodiments 1-47, further comprising selecting the subject to whom the IL-2 conjugate and the anti-PD-1 antibody or antigen-binding fragment thereof are administered at least in part on the basis of the lung cancer being NSCLC having a PD-L1 tumor progession score of less than 50% or of 1-49%.

Embodiment 49 is an IL-2 conjugate for use in the method of any one of embodiments 1-48.

Embodiment 50 is use of an IL-2 conjugate for the manufacture of a medicament for the method of any one of embodiments 1-49.

Embodiment 51 is the method, IL-2 conjugate for use, or use of any of the preceding embodiments, wherein the anti-PD-1 antibody or antigen-binding fragment thereof comprises:
(a) a heavy chain variable region comprising a sequence of amino acids as set forth in SEQ ID NO:19, or a variant of SEQ ID NO:19, and
(b) a light chain variable region comprising a sequence of amino acids as set forth in SEQ ID NO:14, or a variant of SEQ ID NO:14.

Embodiment 52 is the method, IL-2 conjugate for use, or use of any of the preceding embodiments, wherein the anti-PD-1 antibody or antigen-binding fragment thereof is a monoclonal antibody comprising a heavy chain comprising a sequence of amino acids as set forth in SEQ ID NO:20 and a light chain comprising a sequence of amino acids as set forth in SEQ ID NO: 15.

Embodiment 53 is the method, IL-2 conjugate for use, or use of any of the preceding embodiments, wherein the IL-2 conjugate is pegenzileukin.

In some embodiments of any of the methods, compositions, kits and uses described herein, the anti-PD-1 antibody or antigen-binding fragment thereof comprises: (a) a heavy chain variable region comprising a sequence of amino acids as set forth in SEQ ID NO:19, or a variant of SEQ ID NO:19, and (b) a light chain variable region comprising: (i) a sequence of amino acids as set forth in SEQ ID NO:14 or a variant of SEQ ID NO:14. In some embodiments of any of the methods, compositions, kits and uses described herein, the anti-PD-1 antibody or antigen-binding fragment thereof comprises a heavy chain variable region comprising a sequence of amino acids as set forth in SEQ ID NO:19 and a light chain variable region comprising a sequence of amino acids as set forth in SEQ ID NO: 14. In some embodiments of any of the methods, compositions, kits and uses described herein, the anti-PD-1 antibody or antigen-binding fragment thereof is a monoclonal antibody comprising: (a) a heavy chain comprising a sequence of amino acids as set forth in SEQ ID NO:20, or a variant of SEQ ID NO:20, and (b) a light chain comprising a sequence of amino acids as set forth in SEQ ID NO:15 or a variant of SEQ ID NO:15. In some embodiments of any of the methods, compositions, kits and uses described herein, the anti-PD-1 antibody or antigen-binding fragment thereof is a monoclonal antibody comprising a heavy chain comprising a sequence of amino acids as set forth in SEQ ID NO:20 and a light chain comprising a sequence of amino acids as set forth in SEQ ID NO: 15.

In select embodiments of the of any of the methods, compositions, kits and uses described herein, the antibody or antigen-binding fragment is pembrolizumab. In some embodiments of any of the methods and uses described herein, the method or use comprises administering (i) about 200 mg of an anti-PD-1 antibody (e.g., pembrolizumab) or antigen binding fragment thereof to the patient every approximately three weeks or (ii) about 400 mg of an anti-PD-1 antibody (e.g., pembrolizumab), or antigen binding fragment thereof, to the patient every approximately six weeks.

In all of the methods, compositions, kits and uses described herein, the anti-PD-1 antibody or antigen-binding fragment inhibits the binding of PD-L1 to PD-1, and preferably also inhibits the binding of PD-L2 to PD-1. In some embodiments of any of the methods, compositions, kits and uses described herein, the anti-PD-1 antibody or antigen-binding fragment is a monoclonal antibody, which specifically binds to PD-1 and blocks the binding of PD-L1 to PD-1. In particular embodiments of any of the methods and uses described herein, the anti-PD-1 antibody comprises a heavy chain and a light chain, and wherein the heavy and light chains comprise the amino acid sequences shown in FIG. 1 (SEQ ID NO:15 and SEQ ID NO:20).

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows the change in peripheral CD8+ T_{eff} counts in the indicated subjects at specified times following administration of 8 μg/kg IL-2 conjugate and pembrolizumab. Here and elsewhere, designations such as “C1D1” indicate the treatment cycle and day (e.g., treatment cycle 1, day 1). “PRE” indicates the baseline measurement before administration; 24HR indicates 24 hours after administration; and so on.

FIG. 1B shows the change in peak peripheral CD8+ T_{eff} cell expansion following administration of the first dose of IL-2 conjugate and pembrolizumab. Data is normalized to pre-treatment (C1D1) CD8+ T cell count. Listed values indicate median fold changes.

FIG. 1C shows the change in peripheral CD8+ T_{eff} counts in the indicated subjects at specified times following administration of 16 μg/kg IL-2 conjugate and pembrolizumab.

FIG. 2 shows the percentage of CD8+ T_{eff} cells expressing Ki67 in the indicated subjects at specified times following administration of 8 μg/kg IL-2 conjugate and pembrolizumab.

FIG. 3A shows the change in peripheral natural killer (NK) cell counts in the indicated subjects at specified times following administration of 8 μg/kg IL-2 conjugate and pembrolizumab.

FIG. 3B shows the change in peak peripheral NK cell expansion following administration of the first dose of IL-2 conjugate and pembrolizumab. Data is normalized to pre-treatment (C1D1) NK cell count. Listed values indicate median fold changes.

FIG. 3C shows the change in peripheral natural killer (NK) cell counts in the indicated subjects at specified times following administration of 16 μg/kg IL-2 conjugate and pembrolizumab.

FIG. 4 shows the percentage of NK cells expressing Ki67 in the indicated subjects at specified times following administration of 8 μg/kg IL-2 conjugate and pembrolizumab.

FIG. 5A shows the change in peripheral CD4+ T_{reg} counts in the indicated subjects at specified times following administration of 8 μg/kg IL-2 conjugate and pembrolizumab.

FIG. 5B shows the change in peak peripheral CD4+ T_{reg} cell expansion following administration of the first dose of IL-2 conjugate and pembrolizumab. Data is normalized to pre-treatment (C1D1) CD4+ T cell count. Listed values indicate median fold changes.

FIG. 5C shows the change in peripheral CD4+ T_{reg} counts in the indicated subjects at specified times following administration of 16 μg/kg IL-2 conjugate and pembrolizumab.

FIG. 6 shows the percentage of CD4+ T_{reg} cells expressing Ki67 in the indicated subjects at specified times following administration of 8 μg/kg IL-2 conjugate and pembrolizumab.

FIG. 7A shows the change in eosinophil cell counts in the indicated subjects at specified times following administration of 8 μg/kg IL-2 conjugate and pembrolizumab.

FIG. 7B shows the change in peak peripheral eosinophil cell expansion following administration of the first dose of IL-2 conjugate and pembrolizumab. Data is normalized to pre-treatment (C1D1) eosinophil cell count. Listed values indicate median fold changes.

FIG. 7C shows the change in eosinophil cell counts in the indicated subjects at specified times following administration of 16 μg/kg IL-2 conjugate and pembrolizumab.

FIG. 8A shows serum levels of IFN-γ, IL-5, and IL-6 in the indicated subjects at specified times following administration of 8 μg/kg IL-2 conjugate and pembrolizumab.

FIG. 8B shows the serum level of IL-5 following administration of 8 μg/kg IL-2 conjugate and pembrolizumab. BLQ=below limit of quantification. Data is plotted as mean (range BLQ to maximum value).

FIG. 8C shows the serum level of IL-6 following administration of 8 μg/kg IL-2 conjugate and pembrolizumab. BLQ=below limit of quantification. Data is plotted as mean (range BLQ to maximum value).

FIG. 8D shows serum levels of IFN-γ, IL-5, and IL-6 in the indicated subjects at specified times following administration of 16 μg/kg IL-2 conjugate and pembrolizumab.

FIG. 9A and FIG. 9B show mean concentrations of the IL-2 conjugate, administered at a dose of 8 μg/kg with pembrolizumab, after 1 and 2 cycles, respectively.

FIG. 9C and FIG. 9D show mean concentrations of the IL-2 conjugate, administered at a dose of 16 μg/kg with pembrolizumab, after 1 and 2 cycles, respectively.

FIG. 10 shows the change in peripheral CD8+T_{eff} cell counts in the indicated subjects at specified times following administration of 24 μg/kg IL-2 conjugate and pembrolizumab.

FIG. 11 shows the change in peripheral NK cell counts in the indicated subjects at specified times following administration of 24 μg/kg IL-2 conjugate and pembrolizumab.

FIG. 12 shows the change in peripheral CD4+ T_{reg} cell counts in the indicated subjects at specified times following administration of 24 μg/kg IL-2 conjugate and pembrolizumab.

FIG. 13 shows the change in peripheral eosinophil cell counts in the indicated subjects at specified times following administration of 24 μg/kg IL-2 conjugate and pembrolizumab.

FIG. 14A and FIG. 14B show mean concentrations of the IL-2 conjugate, administered at a dose of 24 μg/kg with pembrolizumab, after 1 and 2 cycles, respectively.

FIG. 15 shows the levels of IFN-γ, IL-6, and IL-5 in the indicated subjects treated with 24 μg/kg of the IL-2 conjugate and pembrolizumab at specified times following administration of the IL-2 conjugate.

FIG. 16 shows the change in peripheral CD8+T_{eff} cell counts in the indicated subjects at specified times following administration of 32 μg/kg IL-2 conjugate and pembrolizumab.

FIG. 17 shows the peripheral CD4+ T_{reg} cell counts in the indicated subjects at specified times following administration of 32 μg/kg IL-2 conjugate and pembrolizumab.

FIG. 18A and FIG. 18B show mean concentrations of the IL-2 conjugate, administered at a dose of 32 μg/kg with pembrolizumab, after 1 and 2 cycles, respectively.

FIG. 19 shows the levels of IFN-γ, IL-6, and IL-5 in the indicated subjects treated with 32 μg/kg of the IL-2 conjugate and pembrolizumab at specified times following administration of the IL-2 conjugate.

FIG. 20 shows a graph of anti-tumor activity of Compound A dosed IV on a QWx3 Schedule from Study 1 in Example 3. Black arrows denote days of dosing with Compound A.

FIG. 21 shows a graph of tumor volumes with Compound A dosed IV on a QWx3 Schedule from Study 1 in Example 3.

FIG. 22 shows tumor volumes on Day 15 post treatment for each animal treated QWx3 dosing with Compound A from Study 1 in Example 3. Black arrows denote days of dosing with Compound A.

FIG. 23 shows tumor volumes on Day 15 post treatment for each animal with Q2Wx2 dosing with Compound A from Study 1 in Example 3.

FIG. 24 shows mean tumor growth curves from treatment of mice with vehicle, 6 mg/kg Compound A as a single agent, anti-PD-1 antibody as a single agent, and the combination of 6 mg/kg Compound A and anti-PD-1 antibody from Study 2 of Example 3. Black arrows denote days of dosing with Compound A.

FIG. 25 shows a graph of % TGI data on Day 15 post treatment in the group treated with the combination of Compound A and anti-PD-1 antibody, compared to the groups treated with vehicle, Compound A alone or the anti-PD-1 antibody alone from Study 2 of Example 3. *p<0.05, **p<0.01, and ***p<0.01; vs. vehicle control. ^{⊥}p<0.05 vs. anti-PD-1 antibody. #p<0.05 vs. Compound A. Data represent mean tumor volume±SEM (14 mice/group).

FIG. 26 shows a graph of Kaplan-Meier survival curves for treatment groups from Study 2 of Example 3. *p<0.05 vs. vehicle control. ^{⊥}p<0.05 vs. anti-PD-1 antibody. #p<0.05 vs. Compound A.

FIG. 27 represents mean tumor growth curves when Compound A was dosed a single agent at 1 mg/kg, 3 mg/kg, 6 mg/kg, and 9 mg/kg in Study 3 of Example 3. Data represent mean tumor volume±SEM (14 mice/group; except 12 mice/group for 9 mg/kg Compound A). Black arrows denote days of Compound A dosing.

FIG. 28 represent individual tumor volumes on Day 15 post-treatment from Study 3 of Example 3. Data represent individual tumor volumes; the mean±SEM and % TGI compared to the vehicle control are also displayed. ***p<0.01 vs. vehicle control.

FIG. 29 shows a graph of Kaplan-Meier survival curves for treatment groups treated with vehicle (control), anti-PD-1 antibody alone, Compound A alone, and the combination of Compound A and anti-PD-1 antibody. *p<0.05 vs. vehicle control from Study 3 of Example 3. ^{⊥}p<0.05 vs. anti-PD-1 antibody. #p<0.05 vs. Compound A.

FIG. 30 shows amino acid sequences of the light chain and heavy chain for an exemplary anti-PD-1 monoclonal antibody useful in the present invention (SEQ ID NOs: 15 and 20, respectively). Light chain and heavy chain variable regions are underlined (SEQ ID NOs: 14 and 19) and CDRs are bold and boxed.

### DETAILED DESCRIPTION OF THE DISCLOSURE

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. To the extent any material incorporated herein by reference is inconsistent with the express content of this disclosure, the express content controls. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless the context requires otherwise. Furthermore, use of the term “including” as well as other forms, such as “include”, “includes,” and “included,” is not limiting.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error, such as for example, within 15%, 10%, or 5%.

The section headings used herein are for organizational purposes only and are not to be construed as limiting the subject matter described.

As used herein, the terms “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

As used herein, the term “unnatural amino acid” refers to an amino acid other than one of the 20 naturally occurring amino acids. Exemplary unnatural amino acids are described in Young et al., “Beyond the canonical 20 amino acids: expanding the genetic lexicon,” J of Biological Chemistry 285(15): 11039-11044 (2010), the disclosure of which is incorporated herein by reference.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments. “Parental antibodies” are antibodies obtained by exposure of an immune system to an antigen prior to modification of the antibodies for an intended use, such as humanization of an antibody for use as a human therapeutic.

In general, the basic antibody structural unit comprises a tetramer. Each tetramer 5 includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., ₂^{nd} 15 ed. Raven Press, N.Y. (1989).

The variable regions of each light/heavy chain pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J Biol. Chem. 252:6609-6616; Chothia, et al., (1987) J Mol. Biol. 196:901-917 or Chothia, et al., (1989) Nature 342:878-883.

The term “hypervariable region” refers to the amino acid residues of an antibody that are responsible for antigen-binding. The hypervariable region comprises amino acid residues from a “complementarity determining region” or “CDR” (i.e. CDRL1, CDRL2 and CDRL3 in the light chain variable domain and CDRH1, CDRH2 and CDRH3 in the heavy chain variable domain). See Kabat et al. (1991) Sequences of Proteins of lmmunological Interest, 5^{th} Ed. Public Health Service, National Institutes of Health, Bethesda, Md. (defining the CDR 35 regions of an antibody by sequence); see also Chothia and Lesk (1987) J Mol. Biol. 196: 901-917 (defining the CDR regions of an antibody by structure). The term “framework” or “FR” residues refers to those variable domain residues other than the hypervariable region residues defined herein as CDR residues.

Unless otherwise indicated, an “antibody fragment” or “antigen binding fragment” refers to antigen binding fragments of antibodies, i.e. antibody fragments that retain the ability to specifically bind to the antigen bound by the full-length antibody, e.g. fragments that retain one or more CDR regions. Examples of antibody binding fragments include, but are not limited to, Fab, Fab′, F(ab′)2, and Fv fragments.

An antibody that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g. without producing undesired results such as false positives. Antibodies, or binding fragments thereof, useful in the present invention will bind to the target protein with an affinity that is at least two-fold greater, preferably at least ten times greater, more preferably at least 20-times greater, and most preferably at least 100-times greater than the affinity with non-target proteins. As used herein, an antibody is said to bind specifically to a polypeptide comprising a given amino acid sequence, e.g. the amino acid sequence of a mature human PD-1 or human PD-L1 molecule, if it binds to polypeptides comprising that sequence but does not bind to proteins lacking that sequence.

“CDR” or “CDRs” means complementarity determining region(s) in an immunoglobulin variable region, generally defined using the Kabat numbering system.

“Chothia” means an antibody numbering system described in Al-Lazikani et al., JMB 273:927-948 (1997).

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in the art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 1.

TABLE 1
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Kabat,” as used herein, means an immunoglobulin alignment and numbering system pioneered by Elvin A. Kabat ((1991) Sequences of Proteins of Immunological Interest, 5th Ed. Public Health Service, National Institutes of Health, Bethesda, Md.).

An “anti-PD-1 antibody” useful in the any of the treatment methods, compositions and uses of the present invention include monoclonal antibodies (mAb), or antigen binding fragments thereof, which specifically bind to human PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any of the treatment methods, compositions and uses of the present invention in which a human individual is being treated, the PD-1 antibody or antigen binding fragment thereof is a PD-1 antagonist that blocks binding of human PD-L1 to human PD-1, or blocks binding of both human PD-L1 and PD-L2 to human PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively. An anti-PD-1 antibody may be a human antibody, a humanized antibody or a chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)2, scFv and Fv fragments.

“PD-L1” or “PD-L2” expression means any detectable level of expression of the designated PD-L protein on the cell surface or of the designated PD-L mRNA within a cell or tissue, unless otherwise defined. PD-L protein expression may be detected with a diagnostic PD-L antibody in an IHC assay of a tumor tissue section or by flow cytometry. Alternatively, PD-L protein expression by tumor cells may be detected by PET imaging, using a binding agent (e.g., antibody fragment, affibody and the like) that specifically binds to the desired PD-L target, e.g., PD-L1 or PD-L2. Techniques for detecting and measuring PD-L mRNA expression include RT-PCR and real-time quantitative RT-PCR.

As used herein, “nucleotide” refers to a compound comprising a nucleoside moiety and a phosphate moiety. Exemplary natural nucleotides include, without limitation, adenosine triphosphate (ATP), uridine triphosphate (UTP), cytidine triphosphate (CTP), guanosine triphosphate (GTP), adenosine diphosphate (ADP), uridine diphosphate (UDP), cytidine diphosphate (CDP), guanosine diphosphate (GDP), adenosine monophosphate (AMP), uridine monophosphate (UMP), cytidine monophosphate (CMP), and guanosine monophosphate (GMP), deoxyadenosine triphosphate (dATP), deoxythymidine triphosphate (dTTP), deoxycytidine triphosphate (dCTP), deoxyguanosine triphosphate (dGTP), deoxyadenosine diphosphate (dADP), thymidine diphosphate (dTDP), deoxycytidine diphosphate (dCDP), deoxyguanosine diphosphate (dGDP), deoxyadenosine monophosphate (dAMP), deoxythymidine monophosphate (dTMP), deoxycytidine monophosphate (dCMP), and deoxyguanosine monophosphate (dGMP). Exemplary natural deoxyribonucleotides, which comprise a deoxyribose as the sugar moiety, include dATP, dTTP, dCTP, dGTP, dADP, dTDP, dCDP, dGDP, dAMP, dTMP, dCMP, and dGMP. Exemplary natural ribonucleotides, which comprise a ribose as the sugar moiety, include ATP, UTP, CTP, GTP, ADP, UDP, CDP, GDP, AMP, UMP, CMP, and GMP.

As used herein, “base” or “nucleobase” refers to at least the nucleobase portion of a nucleoside or nucleotide (nucleoside and nucleotide encompass the ribo or deoxyribo variants), which may in some cases contain further modifications to the sugar portion of the nucleoside or nucleotide. In some cases, “base” is also used to represent the entire nucleoside or nucleotide (for example, a “base” may be incorporated by a DNA polymerase into DNA, or by an RNA polymerase into RNA). However, the term “base” should not be interpreted as necessarily representing the entire nucleoside or nucleotide unless required by the context. In the chemical structures provided herein of a base or nucleobase, only the base of the nucleoside or nucleotide is shown, with the sugar moiety and, optionally, any phosphate residues omitted for clarity. As used in the chemical structures provided herein of a base or nucleobase, the wavy line represents connection to a nucleoside or nucleotide, in which the sugar portion of the nucleoside or nucleotide may be further modified. In some embodiments, the wavy line represents attachment of the base or nucleobase to the sugar portion, such as a pentose, of the nucleoside or nucleotide. In some embodiments, the pentose is a ribose or a deoxyribose.

In some embodiments, a nucleobase is generally the heterocyclic base portion of a nucleoside. Nucleobases may be naturally occurring, may be modified, may bear no similarity to natural bases, and/or may be synthesized, e.g., by organic synthesis. In certain embodiments, a nucleobase comprises any atom or group of atoms in a nucleoside or nucleotide, where the atom or group of atoms is capable of interacting with a base of another nucleic acid with or without the use of hydrogen bonds. In certain embodiments, an unnatural nucleobase is not derived from a natural nucleobase. It should be noted that unnatural nucleobases do not necessarily possess basic properties, however, they are referred to as nucleobases for simplicity. In some embodiments, when referring to a nucleobase, a “(d)” indicates that the nucleobase can be attached to a deoxyribose or a ribose, while “d” without parentheses indicates that the nucleobase is attached to deoxyribose.

As used herein, a “nucleoside” is a compound comprising a nucleobase moiety and a sugar moiety. Nucleosides include, but are not limited to, naturally occurring nucleosides (as found in DNA and RNA), abasic nucleosides, modified nucleosides, and nucleosides having mimetic bases and/or sugar groups. Nucleosides include nucleosides comprising any variety of substituents. A nucleoside can be a glycoside compound formed through glycosidic linking between a nucleic acid base and a reducing group of a sugar.

An “analog” of a chemical structure, as the term is used herein, refers to a chemical structure that preserves substantial similarity with the parent structure, although it may not be readily derived synthetically from the parent structure. In some embodiments, a nucleotide analog is an unnatural nucleotide. In some embodiments, a nucleoside analog is an unnatural nucleoside. A related chemical structure that is readily derived synthetically from a parent chemical structure is referred to as a “derivative.”

As used herein, “dose-limiting toxicity” (DLT) is defined as an adverse event occurring within Day 1 through Day 29 (inclusive)±1 day of a treatment cycle that was not clearly or incontrovertibly solely related to an extraneous cause and that meets the criteria set forth in Example 2 for DLT.

As used herein, “severe cytokine release syndrome” refers to level 4 or 5 cytokine release syndrome as described in Teachey et al., Cancer Discov. 2016; 6(6); 664-79, the disclosure of which is incorporated herein by reference.

As used herein, “pembrolizumab” refers to the humanized anti-PD-1 antibody marketed under the brand name “Keytruda” by Merck & Co.

As used herein, “carboplatin” refers to the chemotherapy drug also marketed under the brand name “Novaplus”.

As used herein, “cisplatin” refers to the chemotherapy drug also marketed under the brand name “Platinol-AQ”.

As used herein, “Nab-paclitaxel” refers to the chemotherapy drug also marketed under the brand name “Abraxane”.

As used herein, “Pemetrexed” refers to the chemotherapy drug also marketed under the brand name “Alimta”.

Although various features of the invention may be described in the context of a single embodiment, the features may also be provided separately or in any suitable combination. Conversely, although the invention may be described herein in the context of separate embodiments for clarity, the invention may also be implemented in a single embodiment.

### IL-2 Conjugates

Interleukin 2 (IL-2) is a pleiotropic type-1 cytokine whose structure comprises a 15.5 kDa four α-helix bundle. The precursor form of IL-2 is 153 amino acid residues in length, with the first 20 amino acids forming a signal peptide and residues 21-153 forming the mature form. IL-2 is produced primarily by CD4+ T cells post antigen stimulation and to a lesser extent, by CD8+ cells, Natural Killer (NK) cells, and Natural killer T (NKT) cells, activated dendritic cells (DCs), and mast cells. IL-2 signaling occurs through interaction with specific combinations of IL-2 receptor (IL-2R) subunits, IL-2Rα (also known as CD25), IL-2RP (also known as CD122), and IL-2Rγ (also known as CD132). Interaction of IL-2 with the IL-2Rα forms the “low-affinity” IL-2 receptor complex with a K_{d} of about 10⁻⁸ M. Interaction of IL-2 with IL-2RP and IL-2Rγ forms the “intermediate-affinity” IL-2 receptor complex with a K_{d} of about 10-9 M. Interaction of IL-2 with all three subunits, IL-2Rα, IL-2Rβ, and IL-2Rγ, forms the “high-affinity” IL-2 receptor complex with a K_{d} of about >10⁻¹¹ M.

In some instances, IL-2 signaling via the “high-affinity” IL-2Rαβγ complex modulates the activation and proliferation of regulatory T cells. Regulatory T cells, or CD4⁺CD25⁺Foxp3⁺ regulatory T (Treg) cells, mediate maintenance of immune homeostasis by suppression of effector cells such as CD4⁺ T cells, CD8⁺ T cells, B cells, NK cells, and NKT cells. In some instances, Treg cells are generated from the thymus (tTreg cells) or are induced from naïve T cells in the periphery (pTreg cells). In some cases, Treg cells are considered as the mediator of peripheral tolerance. Indeed, in one study, transfer of CD25-depleted peripheral CD4⁺ T cells produced a variety of autoimmune diseases in nude mice, whereas cotransfer of CD4⁺CD25⁺ T cells suppressed the development of autoimmunity (Sakaguchi, et al., “Immunologic self-tolerance maintained by activated T cells expressing IL-2 receptor alpha-chains (CD25),” J Immunol. 155(3): 1151-1164 (1995), the disclosure of which is incorporated herein by reference). Augmentation of the Treg cell population down-regulates effector T cell proliferation and suppresses autoimmunity and T cell anti-tumor responses.

IL-2 signaling via the “intermediate-affinity” IL-2Rβγ complex modulates the activation and proliferation of CD8+ effector T (T_{eff}) cells, NK cells, and NKT cells. CD8⁺ T_{eff} cells (also known as cytotoxic T cells, Tc cells, cytotoxic T lymphocytes, CTLs, T-killer cells, cytolytic T cells, Tcon, or killer T cells) are T lymphocytes that recognize and kill damaged cells, cancerous cells, and pathogen-infected cells. NK and NKT cells are types of lymphocytes that, similar to CD8⁺ T_{eff} cells, target cancerous cells and pathogen-infected cells.

In some instances, IL-2 signaling is utilized to modulate T cell responses and subsequently for treatment of a cancer. For example, IL-2 is administered in a high-dose form to induce expansion of T_{eff} cell populations for treatment of a cancer. However, high-dose IL-2 further leads to concomitant stimulation of Treg cells that dampen anti-tumor immune responses. High-dose IL-2 also induces toxic adverse events mediated by the engagement of IL-2R alpha chain-expressing cells in the vasculature, including type 2 innate immune cells (ILC-2), eosinophils and endothelial cells. This leads to eosinophilia, capillary leak and vascular leak syndrome (VLS).

Adoptive cell therapy enables physicians to effectively harness a patient's own immune cells to fight diseases such as proliferative disease (e.g., cancer) as well as infectious disease. The effect of IL-2 signaling may be further enhanced by the presence of additional agents or methods in combination therapy. For example, programmed cell death protein 1, also known as PD-1 or CD279, is a cell surface receptor expressed on T cells and pro-B cells which plays a role in regulating the immune system's response to the cells of the human body. PD-1 down-regulates the immune system and promotes self-tolerance by suppressing T cell inflammatory activity. This prevents autoimmune diseases but can also prevent the immune system from killing cancer cells. PD-1 guards against autoimmunity through two mechanisms. First, PD-1 promotes apoptosis (programmed cell death) of antigen-specific T-cells in lymph nodes. Second, PD-1 reduces apoptosis in regulatory T cells (anti-inflammatory, suppressive T cells). Pembrolizumab is a humanized anti-PD-1 antibody that can block PD-1, activate the immune system to attack tumors, and is approved for treatment of certain cancers.

Provided herein are methods of treating a cancer in a subject in need thereof, comprising administering to the subject (a) about 8 μg/kg, 16 μg/kg, 24 μg/kg, or 32 μg/kg of an IL-2 conjugate, and (b) an anti-PD-1 antibody or antigen-binding fragment thereof, wherein the anti-PD-1 antibody or antigen-binding fragment thereof comprises light chain complementarity determining regions (CDRs) comprising a sequence of amino acids as set forth in SEQ ID NOs: 11, 12 and 13 and heavy chain CDRs comprising a sequence of amino acids as set forth in SEQ ID NOs: 16, 17 and 18 (e.g., pembrolizumab).

In some embodiments, the IL-2 sequence comprises the sequence of SEQ ID NO: 1:

(SEQ ID NO: 1)
PTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGS
ETTFMCEYADETATIVEFLNRWITFSQSIISTLT

wherein the amino acid at position P64 is replaced by the structure of Formula (I):

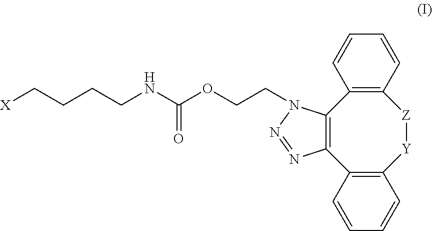

wherein:
Z is CH₂ and Y is

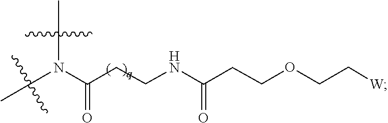

Y is CH₂ and Z is

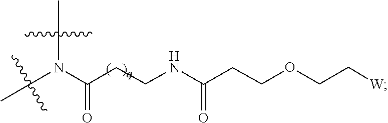

Z is CH₂ and Y is

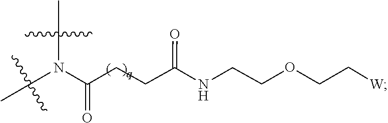

or
Y is CH₂ and Z is

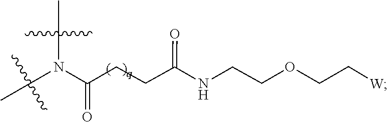

W is a PEG group having an average molecular weight of about 25 kDa-35 kDa;
q is 1, 2, or 3;
X is an L-amino acid having the structure:

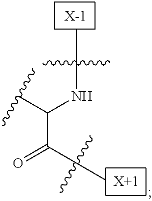

X−1 indicates the point of attachment to the preceding amino acid residue; and
X+1 indicates the point of attachment to the following amino acid residue.

In any of the embodiments or variations of Formula (I) described herein, the IL-2 conjugate is a pharmaceutically acceptable salt, solvate, or hydrate. In some embodiments, the IL-2 conjugate is a pharmaceutically acceptable salt. In some embodiments, the IL-2 conjugate is a solvate. In some embodiments, the IL-2 conjugate is a hydrate.

In any of the embodiments or variations of Formula (I) described herein and pharmaceutical compositions comprising the same, average molecular weight encompasses both weight average molecular weight and number average molecular weight; in other words, for example, both a 30 kDa number average molecular weight and a 30 kDa weight average molecular weight qualify as a 30 kDa molecular weight. In some embodiments, the average molecular weight is weight average molecular weight. In other embodiments, the average molecular weight is number average molecular weight. It is understood that in the methods provided herein, administering an IL-2 conjugate as described herein to a subject comprises administering more than a single molecule of IL-2 conjugate; as such, use of the term “average” to describe the molecular weight of the PEG group refers to the average molecular weight of the PEG groups of the IL-2 conjugate molecules in a dose administered to the subject.

In some embodiments of Formula (I), Z is CH₂ and Y is

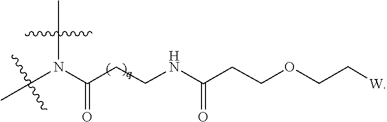

In some embodiments of Formula (I), Y is CH₂ and Z is

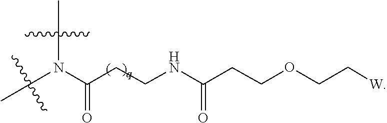

In some embodiments of Formula (I), Z is CH₂ and Y is

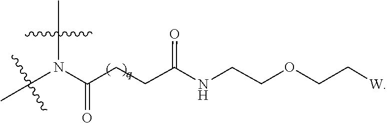

In some embodiments of Formula (I), Y is CH₂ and Z is

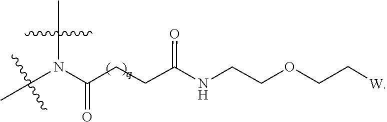

In some embodiments of Formula (I), q is 1. In some embodiments of Formula (I), q is 2. In some embodiments of Formula (I), q is 3.

In some embodiments of Formula (I), W is a PEG group having an average molecular weight of about 25 kDa. In some embodiments of Formula (I), W is a PEG group having an average molecular weight of about 30 kDa. In some embodiments of Formula (I), W is a PEG group having an average molecular weight of about 35 kDa.

In some embodiments of Formula (I), q is 1 and structure of Formula (I) is the structure of Formula (Ia):

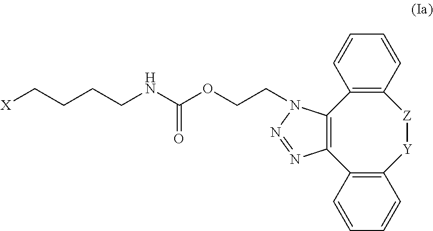

wherein:
Z is CH₂ and Y is

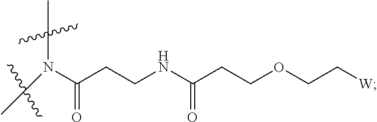

Y is CH₂ and Z is

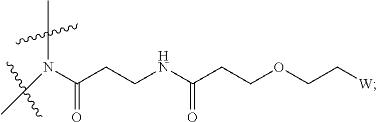

Z is CH₂ and Y is

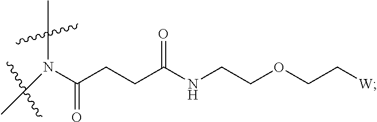

or
Y is CH₂ and Z is

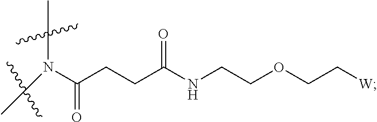

W is a PEG group having an average molecular weight of about 25 kDa-35 kDa;
X is an L-amino acid having the structure:

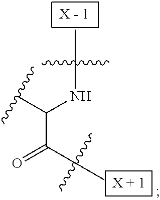 [SMILES: *NC(*)C(*)=O]

X−1 indicates the point of attachment to the preceding amino acid residue; and
X+1 indicates the point of attachment to the following amino acid residue.

In some embodiments of Formula (Ia), Z is CH₂ and Y is

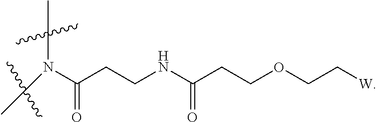

In some embodiments of Formula (Ia), Y is CH₂ and Z is

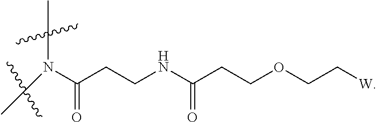

In other embodiments of Formula (Ia), Z is CH₂ and Y is

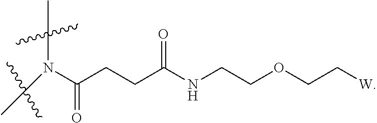

In some embodiments of Formula (Ia), Y is CH₂ and Z is

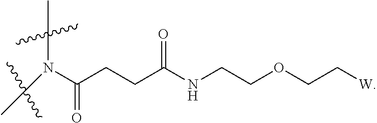

In some embodiments of Formula (Ia), the PEG group has an average molecular weight of about 30 kDa.

In some embodiments, the IL-2 conjugate comprises the sequence of SEQ ID NO: 2:

(SEQ ID NO: 2)
PTSSSTKKTQLQLEHLLLDLQMILNGINNYKNPKLTRMLTFKFYMPKKA
TELKHLQCLEEELK[AzK L1 PEG30KD]LEEVLNLAQSKNFHLRPRD
LISNINVIVLELKGSETTFMCEYADETATIVEFLNRWITFSQSIISTLT

wherein [AzK_L1_PEG30kD] is N6-((2-azidoethoxy)-carbonyl)-L-lysine stably-conjugated to PEG via DBCO-mediated click chemistry to form a compound comprising a structure of Formula (IV) or Formula (V), wherein q is 1 (such as Formula (IVa) or Formula (Va)), and wherein the PEG group has an average molecular weight of about 25-35 kiloDaltons (e.g., about 30 kDa), capped with a methoxy group. The term “DBCO” means a chemical moiety comprising a dibenzocyclooctyne group, such as comprising the mPEG-DBCO compound illustrated in Schemes 1 and 2 of Example 1.

The ratio of regioisomers generated from the click reaction is about 1:1 or greater than 1:1.

PEGs will typically comprise a number of (OCH₂CH₂) monomers (or (CH₂CH₂O) monomers, depending on how the PEG is defined). In some embodiments, the number of (OCH₂CH₂) monomers (or (CH₂CH₂O) monomers) is such that the average molecular weight of the PEG group is about 30 kDa.

In some instances, the PEG is an end-capped polymer, that is, a polymer having at least one terminus capped with a relatively inert group, such as a lower C₁₋₆ alkoxy group, or a hydroxyl group. In some embodiments, the PEG group is a methoxy-PEG (commonly referred to as mPEG), which is a linear form of PEG wherein one terminus of the polymer is a methoxy (—OCH₃) group, and the other terminus is a hydroxyl or other functional group that can be optionally chemically modified.

In some embodiments, the PEG group is a linear or branched PEG group. In some embodiments, the PEG group is a linear PEG group. In some embodiments, the PEG group is a branched PEG group. In some embodiments, the PEG group is a methoxy PEG group. In some embodiments, the PEG group is a linear or branched methoxy PEG group. In some embodiments, the PEG group is a linear methoxy PEG group. In some embodiments, the PEG group is a branched methoxy PEG group. For example, included within the scope of the present disclosure are IL-2 conjugates comprising a PEG group having a molecular weight of 30,000 Da±3,000 Da, or 30,000 Da±4,500 Da, or 30,000 Da±5,000 Da.

In some embodiments, the IL-2 conjugate comprises the amino acid sequence of SEQ ID NO: 1 in which the amino acid residue P64 is replaced by the structure of Formula (IV) or Formula (V), or a mixture of Formula (IV) and Formula (V):

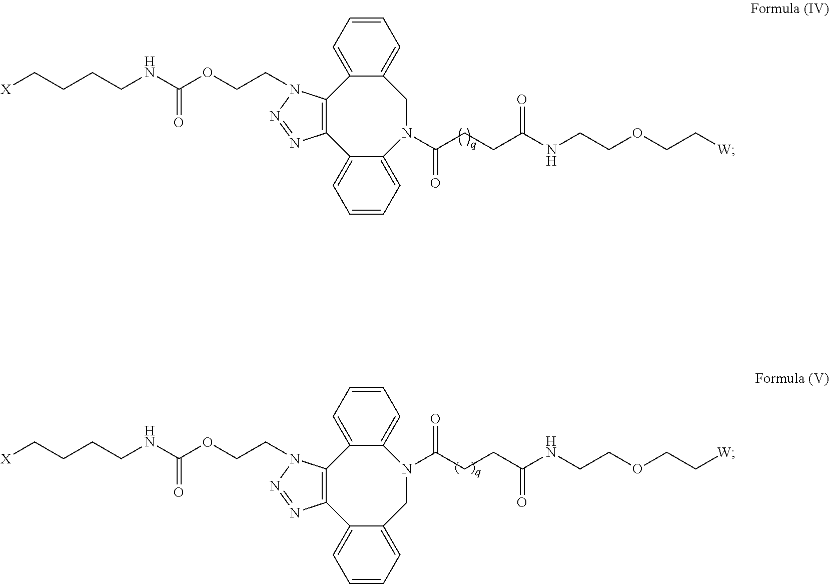

wherein:
W is a PEG group having an average molecular weight of about 25 kDa-35 kDa;
q is 1, 2, or 3; and
X has the structure:

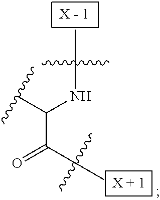 [SMILES: *NC(*)C(*)=O]

X−1 indicates the point of attachment to the preceding amino acid residue; and
X+1 indicates the point of attachment to the following amino acid residue.

In some embodiments of Formula (IV) or Formula (V), or a mixture of Formula (IV) or Formula (V), q is 1. In some embodiments of Formula (IV) or Formula (V), or a mixture of Formula (IV) or Formula (V), q is 2. In some embodiments of Formula (IV) or Formula (V), or a mixture of Formula (IV) or Formula (V), q is 3.

In some embodiments of Formula (IV) or Formula (V), or a mixture of Formula (IV) or Formula (V), W is a PEG group having an average molecular weight of about 25 kDa. In some embodiments of Formula (IV) or Formula (V), or a mixture of Formula (IV) or Formula (V), W is a PEG group having an average molecular weight of about 30 kDa. In some embodiments of Formula (IV) or Formula (V), or a mixture of Formula (IV) or Formula (V), W is a PEG group having an average molecular weight of about 35 kDa.

In any of the embodiments described herein, the structure of Formula (I) has the structure of Formula (IV) or Formula (V), or is a mixture of Formula (IV) and Formula (V). In some embodiments, the structure of Formula (I) has the structure of Formula (IV). In some embodiments, the structure of Formula (I) has the structure of Formula (V). In some embodiments, the structure of Formula (I) is a mixture of Formula (IV) and Formula (V).

In some embodiments of Formula (IV) or Formula (V), or a mixture of Formula (IV) and Formula (V), q is 1, the structure of Formula (IV) is the structure of Formula (IVa), and the structure of Formula (V) is the structure of Formula (Va):

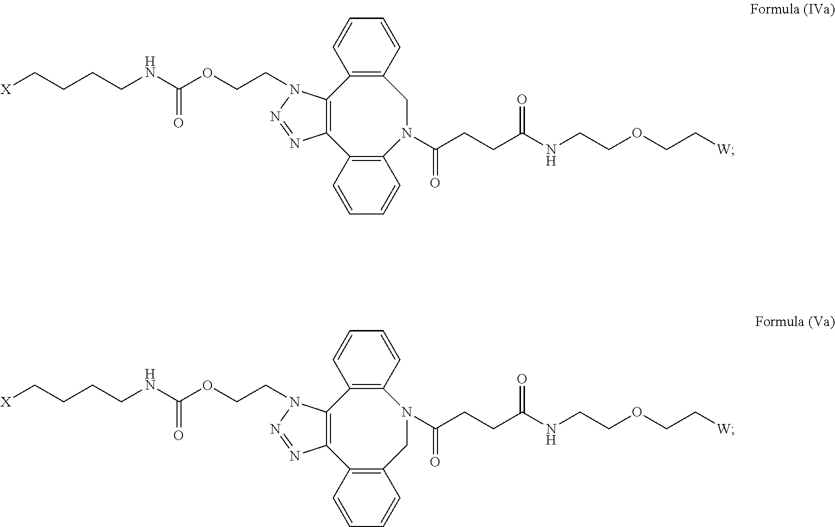

wherein:
W is a PEG group having an average molecular weight of about 25 kDa-35 kDa; and
X has the structure:

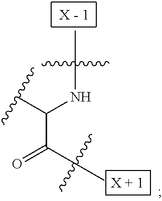 [SMILES: *NC(*)C(*)=O]

X−1 indicates the point of attachment to the preceding amino acid residue; and
X+1 indicates the point of attachment to the following amino acid residue.

In some embodiments of Formula (IVa) or Formula (Va), or a mixture of Formula (IVa) and Formula (Va), the PEG group has an average molecular weight of about 30 kDa.

In any of the embodiments described herein, the structure of Formula (I) has the structure of Formula (IVa) or Formula (Va), or is a mixture of Formula (IVa) and Formula (Va). In some embodiments, the structure of Formula (I) has the structure of Formula (IVa). In some embodiments, the structure of Formula (I) has the structure of Formula (Va). In some embodiments, the structure of Formula (I) is a mixture of Formula (IVa) and Formula (Va).

In some embodiments, the IL-2 conjugate comprises the amino acid sequence of SEQ ID NO: 1 in which the amino acid residue P64 is replaced by the structure of Formula (XII) or Formula (XIII), or a mixture of Formula (XII) and Formula (XIII):

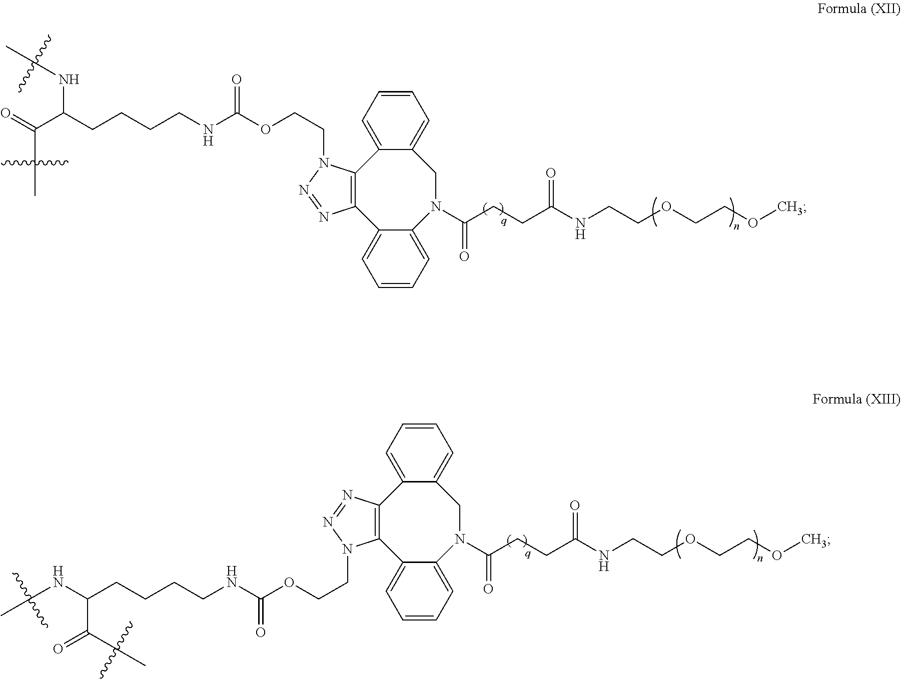

wherein:
n is an integer such that —(OCH₂CH₂)ₙ—OCH₃ has a molecular weight of about 25 kDa-35 kDa;
q is 1, 2, or 3; and
the wavy lines indicate convalent bonds to amino acid residues within SEQ ID NO: 1 that are not replaced.

In some embodiments of Formula (XII) or Formula (XIII), or a mixture of Formula (XII) and Formula (XIII), q is 1. In some embodiments of Formula (XII) or Formula (XIII), or a mixture of Formula (XII) and Formula (XIII), q is 2. In some embodiments of Formula (XII) or Formula (XIII), or a mixture of Formula (XII) and Formula (XIII), q is 3.

In some embodiments of Formula (XII) or Formula (XIII), or a mixture of Formula (XII) and Formula (XIII), n is an integer such that —(OCH₂CH₂)ₙ—OCH₃ has a molecular weight of about 30 kDa.

In any of the embodiments described herein, the structure of Formula (I) has the structure of Formula (XII) or Formula (XIII), or is a mixture of Formula (XII) and Formula (XIII). In some embodiments, the structure of Formula (I) has the structure of Formula (XII). In some embodiments, the structure of Formula (I) has the structure of Formula (XIII). In some embodiments, the structure of Formula (I) is a mixture of Formula (XII) and Formula (XIII).

In some embodiments of Formula (XII) or Formula (XIII), or a mixture of Formula (XII) and Formula (XIII), q is 1, the structure of Formula (XII) is the structure of Formula (XIIa), and the structure of Formula (XIII) is the structure of Formula (XIIIa):

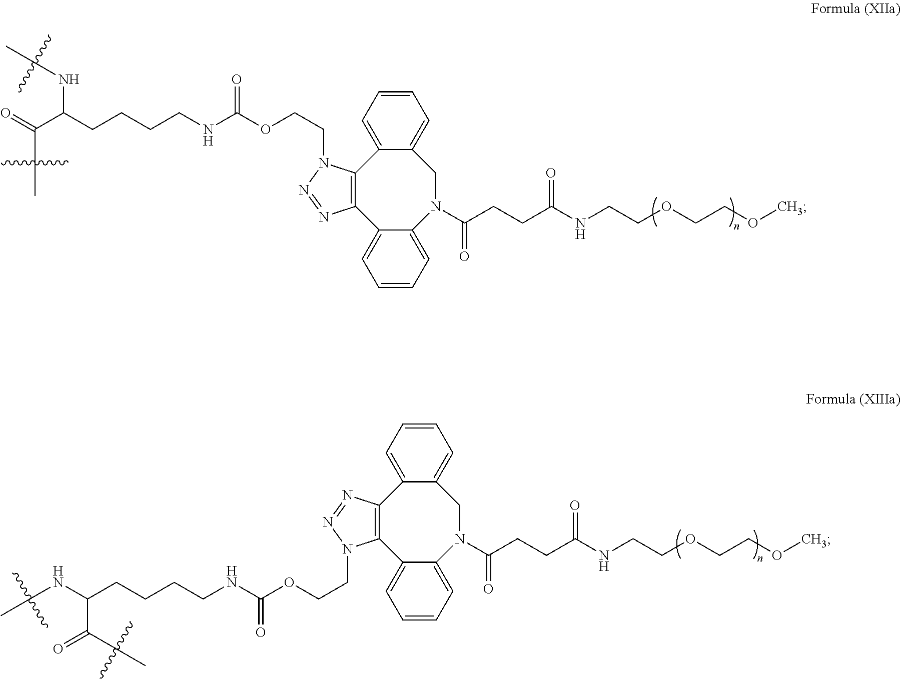

wherein:
n is an integer such that —(OCH₂CH₂)ₙ—OCH₃ has a molecular weight of about 25 kDa-35 kDa; and
the wavy lines indicate convalent bonds to amino acid residues within SEQ ID NO: 1 that are not replaced.

In some embodiments of Formula (XIIa) or Formula (XIIIa), or a mixture of Formula (XIIa) and Formula (XIIIa), n is an integer such that —(OCH₂CH₂)ₙ—OCH₃ has a molecular weight of about 30 kDa.

In any of the embodiments described herein, the structure of Formula (I) has the structure of Formula (XIIa) or Formula (XIIIa), or is a mixture of Formula (XIIa) and Formula (XIIIa). In some embodiments, the structure of Formula (I) has the structure of Formula (XIIa). In some embodiments, the structure of Formula (I) has the structure of Formula (XIIIa). In some embodiments, the structure of Formula (I) is a mixture of Formula (XIIa) and Formula (XIIIa).

In some embodiments, the IL-2 conjugate comprises the amino acid sequence of SEQ ID NO: 1 in which the amino acid residue P64 is replaced by the structure of Formula (XIV) or Formula (XV), or a mixture of Formula (XIV) and Formula (XV):

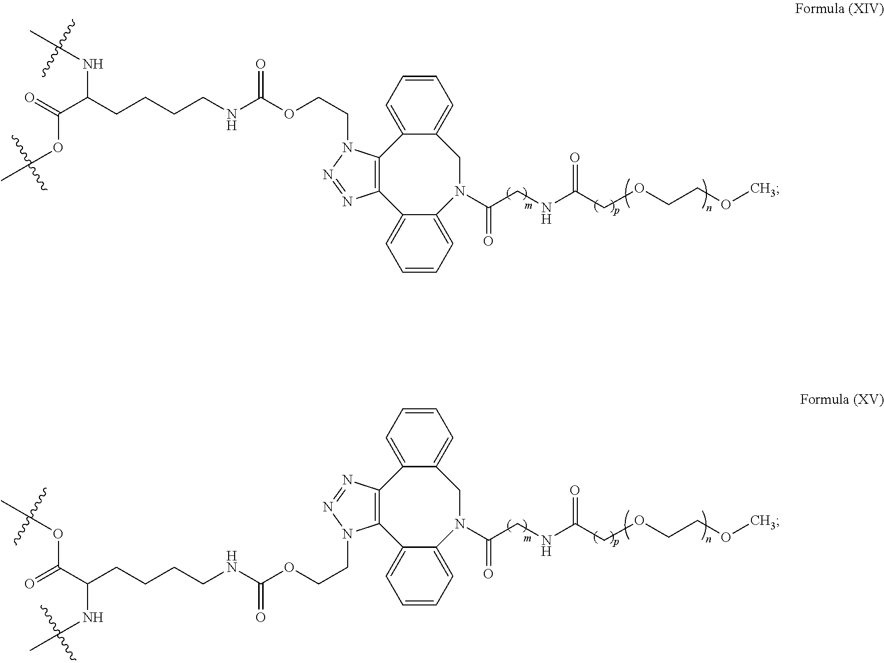

wherein:
m is an integer from 0 to 20;
p is an integer from 0 to 20;
n is an integer such that the PEG group has an average molecular weight of about 25 kDa-35 kDa; and
the wavy lines indicate covalent bonds to amino acid residues within SEQ ID NO: 1 that are not replaced.

In some embodiments of Formula (XIV) or Formula (XV), or a mixture of Formula (XIV) and Formula (XV), n is an integer such that the PEG group has an average molecular weight of about 30 kDa.

In some embodiments, m is an integer from 0 to 15. In some embodiments, m is an integer from 0 to 10. In some embodiments, m is an integer from 0 to 5. In some embodiments, m is an integer from 1 to 5. In some embodiments, m is 1. In some embodiments, m is 2. In some embodiments, m is 3. In some embodiments, m is 4. In some embodiments, m is 5.

In some embodiments, p is an integer from 0 to 15. In some embodiments, p is an integer from 0 to 10. In some embodiments, p is an integer from 0 to 5. In some embodiments, p is an integer from 1 to 5. In some embodiments, p is 1. In some embodiments, p is 2. In some embodiments, p is 3. In some embodiments, p is 4. In some embodiments, p is 5.

In some embodiments, m and p are each 2.

In any of the embodiments described herein, the structure of Formula (I) has the structure of Formula (XIV) or Formula (XV), or is a mixture of Formula (XIV) and Formula (XV). In some embodiments, the structure of Formula (I) has the structure of Formula (XIV). In some embodiments, the structure of Formula (I) has the structure of Formula (XV). In some embodiments, the structure of Formula (I) is a mixture of Formula (XIV) and Formula (XV).

In some embodiments, the IL-2 conjugate comprises the amino acid sequence of SEQ ID NO: 1 in which the amino acid residue P64 is replaced by the structure of Formula (XVI) or Formula (XVII), or a mixture of Formula (XVI) and Formula (XVII):

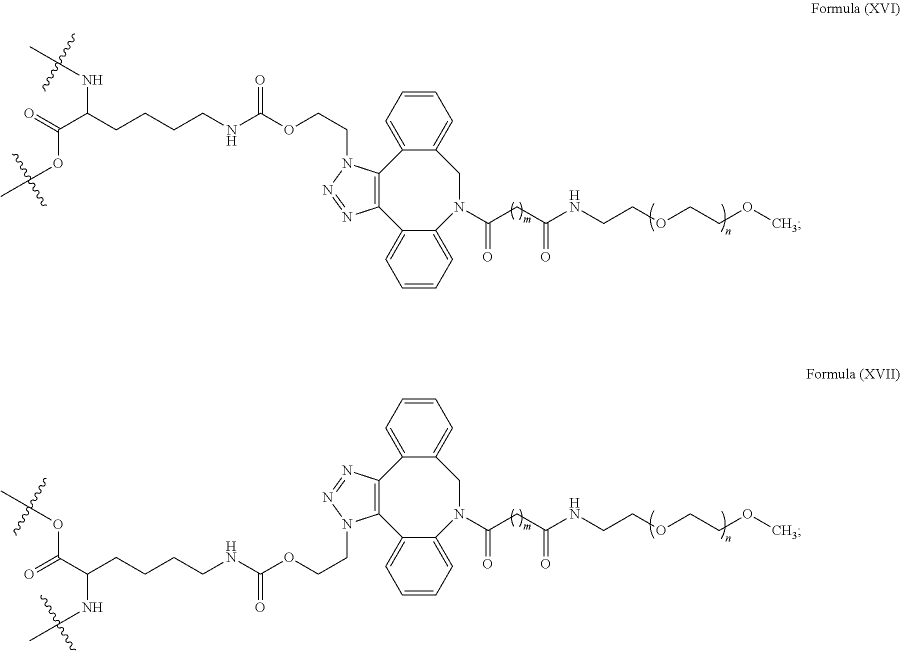

wherein:
m is an integer from 0 to 20;
n is an integer such that the PEG group has an average molecular weight of about 25 kDa-35 kDa; and
the wavy lines indicate covalent bonds to amino acid residues within SEQ ID NO: 1 that are not replaced.

In some embodiments of Formula (XVI) or Formula (XVII), or a mixture of Formula (XVI) and Formula (XVII), n is an integer such that the PEG group has an average molecular weight of about 30 kDa.

In some embodiments, m is an integer from 0 to 15. In some embodiments, m is an integer from 0 to 10. In some embodiments, m is an integer from 0 to 5. In some embodiments, m is an integer from 1 to 5. In some embodiments, m is 1. In some embodiments, m is 2. In some embodiments, m is 3. In some embodiments, m is 4. In some embodiments, m is 5.

In any of the embodiments described herein, the structure of Formula (I) has the structure of Formula (XVI) or Formula (XVII), or is a mixture of Formula (XVI) and Formula (XVII). In some embodiments, the structure of Formula (I) has the structure of Formula (XVI). In some embodiments, the structure of Formula (I) has the structure of Formula (XVII). In some embodiments, the structure of Formula (I) is a mixture of Formula (XVI) and Formula (XVII).

### Conjugation Chemistry

In some embodiments, the IL-2 conjugates described herein can be prepared by a conjugation reaction comprising a 1,3-dipolar cycloaddition reaction. In some embodiments, the 1,3-dipolar cycloaddition reaction comprises reaction of an azide and an alkyne (“Click” reaction). In some embodiments, a conjugation reaction described herein comprises the reaction outlined in Scheme I, wherein X is an unnatural amino acid at position P64 of SEQ ID NO: 1.

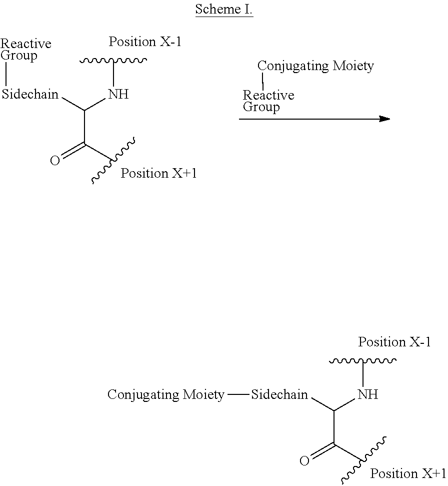

In some embodiments, the conjugating moiety comprises a PEG group as described herein. In some embodiments, a reactive group comprises an alkyne or azide.

In some embodiments, a conjugation reaction described herein comprises the reaction outlined in Scheme II, wherein X is an unnatural amino acid at position P64 of SEQ ID NO: 1.

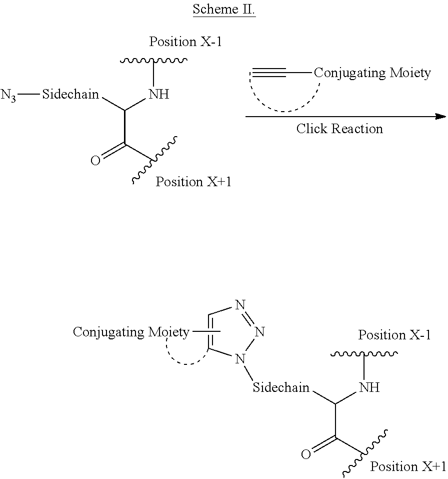

In some embodiments, a conjugation reaction described herein comprises the reaction outlined in Scheme III, wherein X is an unnatural amino acid at position P64 of SEQ ID NO: 1.

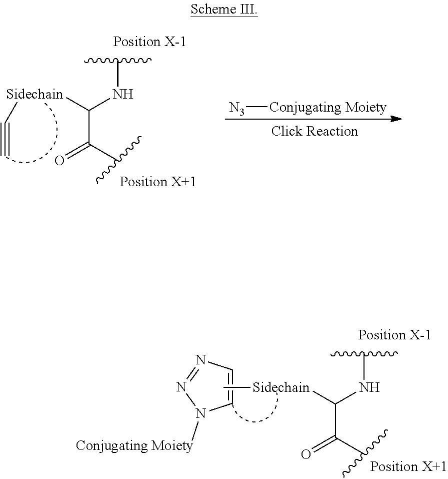

In some embodiments a conjugation reaction described herein comprises the reaction outlined in Scheme IV, wherein X is an unnatural amino acid at position P64 of SEQ ID NO: 1.

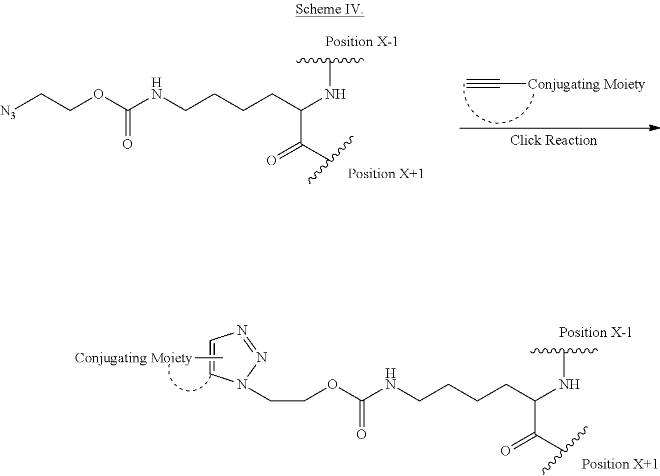

In some embodiments, a conjugation reaction described herein comprises a cycloaddition reaction between an azide moiety, such as that contained in a protein containing an amino acid residue derived from N6-((2-azidoethoxy)-carbonyl)-L-lysine (AzK), and a strained cycloalkyne, such as that derived from DBCO, which is a chemical moiety comprising a dibenzocyclooctyne group. PEG groups comprising a DBCO moiety are commercially available or may be prepared by methods known to those of ordinary skill in the art. Exemplary reactions are shown in Schemes V and VI.

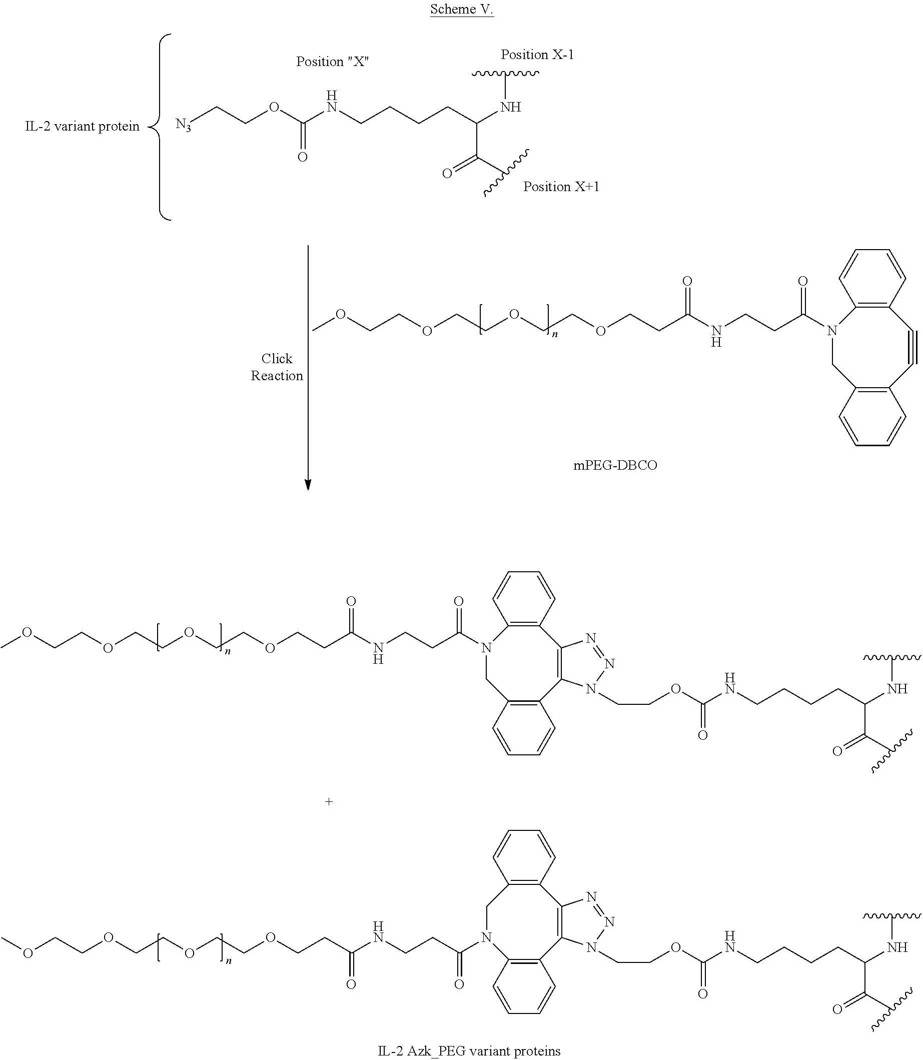

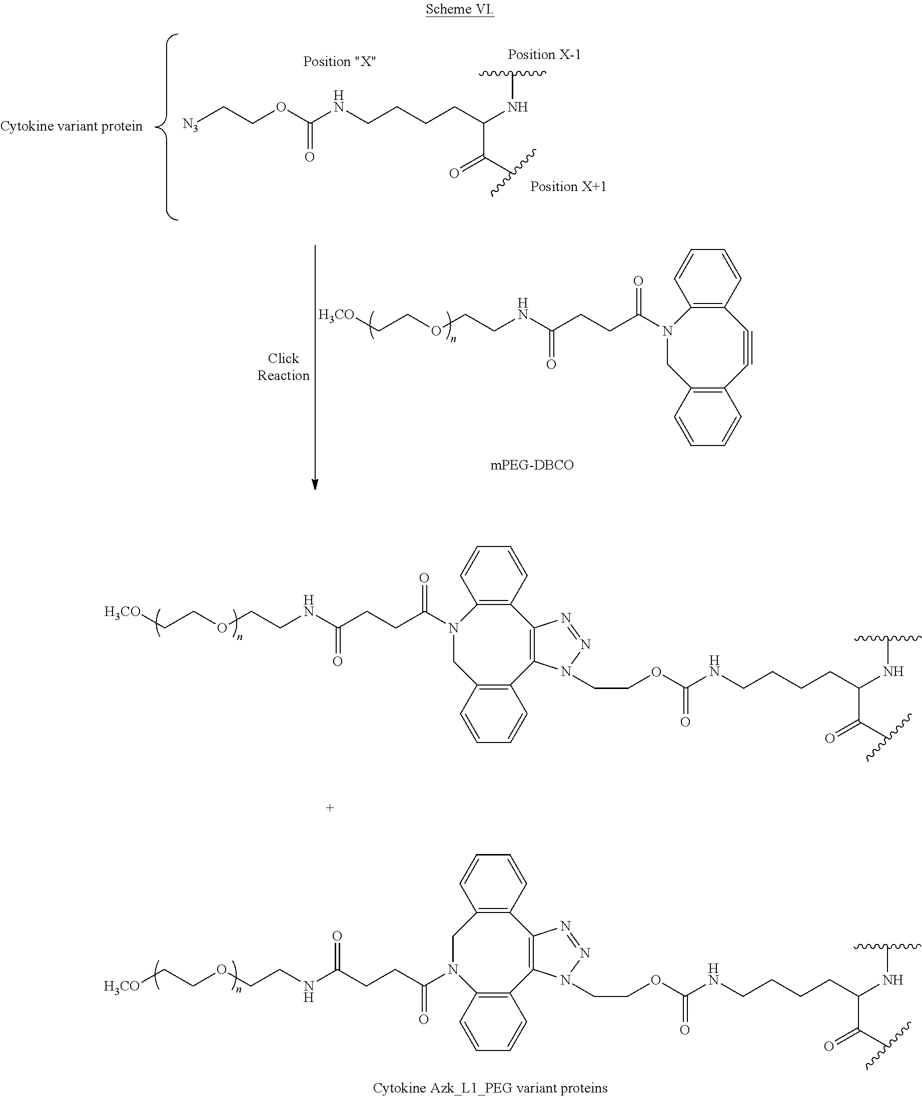

Conjugation reactions such as a click reaction described herein may generate a single regioisomer, or a mixture of regioisomers. In some instances the ratio of regioisomers is about 1:1. In some instances the ratio of regioisomers is about 2:1. In some instances the ratio of regioisomers is about 1.5:1. In some instances the ratio of regioisomers is about 1.2:1. In some instances the ratio of regioisomers is about 1.1:1. In some instances the ratio of regioisomers is greater than 1:1.

### IL-2 Polypeptide Production

In some instances, the IL-2 conjugates described herein, either containing a natural amino acid mutation or an unnatural amino acid mutation, are generated recombinantly or are synthesized chemically. In some instances, IL-2 conjugates described herein are generated recombinantly, for example, either by a host cell system, or in a cell-free system.

In some instances, IL-2 conjugates are generated recombinantly through a host cell system. In some cases, the host cell is a eukaryotic cell (e.g., mammalian cell, insect cells, yeast cells or plant cell) or a prokaryotic cell (e.g., Gram-positive bacterium or a Gram-negative bacterium). In some cases, a eukaryotic host cell is a mammalian host cell. In some cases, a mammalian host cell is a stable cell line, or a cell line that has incorporated a genetic material of interest into its own genome and has the capability to express the product of the genetic material after many generations of cell division. In other cases, a mammalian host cell is a transient cell line, or a cell line that has not incorporated a genetic material of interest into its own genome and does not have the capability to express the product of the genetic material after many generations of cell division.

Exemplary mammalian host cells include 293T cell line, 293A cell line, 293FT cell line, 293F cells, 293 H cells, A549 cells, MDCK cells, CHO DG44 cells, CHO-S cells, CHO-K1 cells, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, and T-REx™-HeLa cell line.

In some embodiments, a eukaryotic host cell is an insect host cell. Exemplary insect host cells include Drosophila S2 cells, Sf9 cells, Sf21 cells, High Five™ cells, and expresSF+® cells.

In some embodiments, a eukaryotic host cell is a yeast host cell. Exemplary yeast host cells include Pichia pastoris (K. phaffii) yeast strains such as GS115, KM71H, SMD1168, SMD1168H, and X-33, and Saccharomyces cerevisiae yeast strain such as INVSc1.

In some embodiments, a eukaryotic host cell is a plant host cell. In some instances, the plant cells comprise a cell from algae. Exemplary plant cell lines include strains from Chlamydomonas reinhardtii 137c, or Synechococcus elongatus PPC 7942.

In some embodiments, a host cell is a prokaryotic host cell. Exemplary prokaryotic host cells include BL21, Mach1™, DH10B™, TOP10, DH5α, DH10Bac™, OmniMax™, MegaX™, DH12S™, INV110, TOP10F′, INVαF, TOP10/P3, ccdB Survival, PIR1, PIR2, Stbl2™, Stbl3™, or Stbl4™.

In some instances, suitable polynucleic acid molecules or vectors for the production of an IL-2 polypeptide described herein include any suitable vectors derived from either a eukaryotic or prokaryotic source. Exemplary polynucleic acid molecules or vectors include vectors from bacteria (e.g., E. coli), insects, yeast (e.g., Pichia pastoris, K. phaffii), algae, or mammalian source. Bacterial vectors include, for example, pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Insect vectors include, for example, pFastBac1, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

Yeast vectors include, for example, Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichia pastoris (K. phaffii) vector, pGAPZA, B, & C Pichia pastoris (K. phaffii) vector, pPIC3.5K. Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Algae vectors include, for example, pChlamy-4 vector or MCS vector.

Mammalian vectors include, for example, transient expression vectors or stable expression vectors. Exemplary mammalian transient expression vectors include p3xFLAG-CMV 8, pFLAG-Myc-CMV 19, pFLAG-Myc-CMV 23, pFLAG-CMV 2, pFLAG-CMV 6a, b, c, pFLAG-CMV 5.1, pFLAG-CMV 5a, b, c, p3xFLAG-CMV 7.1, pFLAG-CMV 20, p3xFLAG-Myc-CMV 24, pCMV-FLAG-MAT1, pCMV-FLAG-MAT2, pBICEP-CMV 3, or pBICEP-CMV 4. Exemplary mammalian stable expression vectors include pFLAG-CMV 3, p3xFLAG-CMV 9, p3xFLAG-CMV 13, pFLAG-Myc-CMV 21, p3xFLAG-Myc-CMV 25, pFLAG-CMV 4, p3xFLAG-CMV 10, p3xFLAG-CMV 14, pFLAG-Myc-CMV 22, p3xFLAG-Myc-CMV 26, pBICEP-CMV 1, or pBICEP-CMV 2.

In some instances, a cell-free system is used for the production of an IL-2 polypeptide described herein. In some cases, a cell-free system comprises a mixture of cytoplasmic and/or nuclear components from a cell and is suitable for in vitro nucleic acid synthesis. In some instances, a cell-free system utilizes prokaryotic cell components. In other instances, a cell-free system utilizes eukaryotic cell components. Nucleic acid synthesis is obtained in a cell-free system based on, for example, Drosophila cell, Xenopus egg, Archaea, or HeLa cells. Exemplary cell-free systems include E. coli S30 Extract system, E. coli T7 S30 system, or PURExpress®, XpressCF, and XpressCF+.

Cell-free translation systems variously comprise components such as plasmids, mRNA, DNA, tRNAs, synthetases, release factors, ribosomes, chaperone proteins, translation initiation and elongation factors, natural and/or unnatural amino acids, and/or other components used for protein expression. Such components are optionally modified to improve yields, increase synthesis rate, increase protein product fidelity, or incorporate unnatural amino acids. In some embodiments, cytokines described herein are synthesized using cell-free translation systems described in U.S. Pat. No. 8,778,631; US 2017/0283469; US 2018/0051065; US 2014/0315245; or U.S. Pat. No. 8,778,631, the disclosure of each of which is incorporated herein by reference. In some embodiments, cell-free translation systems comprise modified release factors, or even removal of one or more release factors from the system. In some embodiments, cell-free translation systems comprise a reduced protease concentration. In some embodiments, cell-free translation systems comprise modified tRNAs with re-assigned codons used to code for unnatural amino acids. In some embodiments, the synthetases described herein for the incorporation of unnatural amino acids are used in cell-free translation systems. In some embodiments, tRNAs are pre-loaded with unnatural amino acids using enzymatic or chemical methods before being added to a cell-free translation system. In some embodiments, components for a cell-free translation system are obtained from modified organisms, such as modified bacteria, yeast, or other organism.

In some embodiments, an IL-2 polypeptide is generated as a circularly permuted form, either via an expression host system or through a cell-free system.

### Production of Cytokine Polypeptide Comprising an Unnatural Amino Acid

An orthogonal or expanded genetic code can be used in the present disclosure, in which one or more specific codons present in the nucleic acid sequence of an IL-2 polypeptide are allocated to encode the unnatural amino acid so that it can be genetically incorporated into the IL-2 by using an orthogonal tRNA synthetase/tRNA pair. The orthogonal tRNA synthetase/tRNA pair is capable of charging a tRNA with an unnatural amino acid and is capable of incorporating that unnatural amino acid into the polypeptide chain in response to the codon.

In some instances, the codon is the codon amber, ochre, opal or a quadruplet codon. In some cases, the codon corresponds to the orthogonal tRNA which will be used to carry the unnatural amino acid. In some cases, the codon is amber. In other cases, the codon is an orthogonal codon.

In some instances, the codon is a quadruplet codon, which can be decoded by an orthogonal ribosome ribo-Q1. In some cases, the quadruplet codon is as illustrated in Neumann, et al., “Encoding multiple unnatural amino acids via evolution of a quadruplet-decoding ribosome,” Nature, 464(7287): 441-444 (2010), the disclosure of which is incorporated herein by reference.

In some instances, a codon used in the present disclosure is a recoded codon, e.g., a synonymous codon or a rare codon that is replaced with alternative codon. In some cases, the recoded codon is as described in Napolitano, et al., “Emergent rules for codon choice elucidated by editing rare arginine codons in Escherichia coli,” PNAS, 113(38): E5588-5597 (2016), the disclosure of which is incorporated herein by reference. In some cases, the recoded codon is as described in Ostrov et al., “Design, synthesis, and testing toward a 57-codon genome,” Science 353(6301): 819-822 (2016), the disclosure of which is incorporated herein by reference.

In some instances, unnatural nucleic acids are utilized leading to incorporation of one or more unnatural amino acids into the IL-2. Exemplary unnatural nucleic acids include, but are not limited to, uracil-5-yl, hypoxanthin-9-yl (I), 2-aminoadenin-9-yl, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifiuoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Certain unnatural nucleic acids, such as 5-substituted pyrimidines, 6-azapyrimidines and N-2 substituted purines, N-6 substituted purines, 0-6 substituted purines, 2-aminopropyladenine, 5-propynyluracil, 5-propynylcytosine, 5-methylcytosine, those that increase the stability of duplex formation, universal nucleic acids, hydrophobic nucleic acids, promiscuous nucleic acids, size-expanded nucleic acids, fluorinated nucleic acids, 5-substituted pyrimidines, 6-azapyrimidines and N-2, N-6 and 0-6 substituted purines, including 2-aminopropyladenine, 5-propynyluracil and 5-propynylcytosine. 5-methylcytosine (5-me-C), 5-hydroxymethyl cytosine, xanthine, hypoxanthine, 2-aminoadenine, 6-methyl, other alkyl derivatives of adenine and guanine, 2-propyl and other alkyl derivatives of adenine and guanine, 2-thiouracil, 2-thiothymine and 2-thiocytosine, 5-halouracil, 5-halocytosine, 5-propynyl (—C≡C—CH₃) uracil, 5-propynyl cytosine, other alkynyl derivatives of pyrimidine nucleic acids, 6-azo uracil, 6-azo cytosine, 6-azo thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl, other 5-substituted uracils and cytosines, 7-methylguanine, 7-methyladenine, 2-F-adenine, 2-amino-adenine, 8-azaguanine, 8-azaadenine, 7-deazaguanine, 7-deazaadenine, 3-deazaguanine, 3-deazaadenine, tricyclic pyrimidines, phenoxazine cytidine([5,4-b][1,4]benzoxazin-2(3H)-one), phenothiazine cytidine (1H-pyrimido[5,4-b][1,4]benzothiazin-2(3H)-one), G-clamps, phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one), those in which the purine or pyrimidine base is replaced with other heterocycles, 7-deaza-adenine, 7-deazaguanosine, 2-aminopyridine, 2-pyridone, azacytosine, 5-bromocytosine, bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 2-amino-adenine, 6-thio-guanine, 2-thio-thymine, 4-thio-thymine, 5-propynyl-uracil, 4-thio-uracil, N4-ethylcytosine, 7-deazaguanine, 7-deaza-8-azaguanine, 5-hydroxycytosine, 2′-deoxyuridine, 2-amino-2′-deoxyadenosine, and those described in U.S. Pat. Nos. 3,687,808; 4,845,205; 4,910,300; 4,948,882; 5,093,232; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,645,985; 5,681,941; 5,750,692; 5,763,588; 5,830,653 and 6,005,096; WO 99/62923; Kandimalla et al., (2001) Bioorg. Med. Chem. 9:807-813; The Concise Encyclopedia of Polymer Science and Engineering, Kroschwitz, JI., Ed., John Wiley & Sons, 1990, 858-859; Englisch et al., Angewandte Chemie, International Edition, 1991, 30, 613; and Sanghvi, Chapter 15, Antisense Research and Applications, Crooke and Lebleu Eds., CRC Press, 1993, 273-288. Additional base modifications can be found, for example, in U.S. Pat. No. 3,687,808; Englisch et al., Angewandte Chemie, International Edition, 1991, 30, 613; and Sanghvi, Chapter 15, Antisense Research and Applications, pages 289-302, Crooke and Lebleu ed., CRC Press, 1993; the disclosure of each of which is incorporated herein by reference.

Unnatural nucleic acids comprising various heterocyclic bases and various sugar moieties (and sugar analogs) are available in the art, and the nucleic acids in some cases include one or several heterocyclic bases other than the principal five base components of naturally-occurring nucleic acids. For example, the heterocyclic base includes, in some cases, uracil-5-yl, cytosin-5-yl, adenin-7-yl, adenin-8-yl, guanin-7-yl, guanin-8-yl, 4-aminopyrrolo [2,3-d]pyrimidin-5-yl, 2-amino-4-oxopyrolo [2,3-d] pyrimidin-5-yl, 2-amino-4-oxopyrrolo [2,3-d]pyrimidin-3-yl groups, where the purines are attached to the sugar moiety of the nucleic acid via the 9-position, the pyrimidines via the 1-position, the pyrrolopyrimidines via the 7-position and the pyrazolopyrimidines via the 1-position.

In some embodiments, nucleotide analogs are also modified at the phosphate moiety. Modified phosphate moieties include, but are not limited to, those with modification at the linkage between two nucleotides and contains, for example, a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkage between two nucleotides are through a 3′-5′ linkage or a 2′-5′ linkage, and the linkage contains inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050; the disclosure of each of which is incorporated herein by reference.

In some embodiments, unnatural nucleic acids include 2′,3′-dideoxy-2′,3′-didehydro-nucleosides (PCT/US2002/006460), 5′-substituted DNA and RNA derivatives (PCT/US2011/033961; Saha et al., J. Org Chem., 1995, 60, 788-789; Wang et al., Bioorganic & Medicinal Chemistry Letters, 1999, 9, 885-890; and Mikhailov et al., Nucleosides & Nucleotides, 1991, 10 (1-3), 339-343; Leonid et al., 1995, 14 (3-5), 901-905; and Eppacher et al., Helvetica Chimica Acta, 2004, 87, 3004-3020; PCT/JP2000/004720; PCT/JP2003/002342; PCT/JP2004/013216; PCT/JP2005/020435; PCT/JP2006/315479; PCT/JP2006/324484; PCT/JP2009/056718; PCT/JP2010/067560), or 5′-substituted monomers made as the monophosphate with modified bases (Wang et al., Nucleosides Nucleotides & Nucleic Acids, 2004, 23 (1 & 2), 317-337); the disclosure of each of which is incorporated herein by reference.

In some embodiments, unnatural nucleic acids include modifications at the 5′-position and the 2′-position of the sugar ring (PCT/US94/02993), such as 5′-CH₂-substituted 2′-O-protected nucleosides (Wu et al., Helvetica Chimica Acta, 2000, 83, 1127-1143 and Wu et al., Bioconjugate Chem. 1999, 10, 921-924). In some cases, unnatural nucleic acids include amide linked nucleoside dimers have been prepared for incorporation into oligonucleotides wherein the 3′ linked nucleoside in the dimer (5′ to 3′) comprises a 2′-OCH₃ and a 5′-(S)—CH₃ (Mesmaeker et al., Synlett, 1997, 1287-1290). Unnatural nucleic acids can include 2′-substituted 5′-CH₂ (or O) modified nucleosides (PCT/US92/01020). Unnatural nucleic acids can include 5′-methylenephosphonate DNA and RNA monomers, and dimers (Bohringer et al., Tet. Lett., 1993, 34, 2723-2726; Collingwood et al., Synlett, 1995, 7, 703-705; and Hutter et al., Helvetica Chimica Acta, 2002, 85, 2777-2806). Unnatural nucleic acids can include 5′-phosphonate monomers having a 2′-substitution (US2006/0074035) and other modified 5′-phosphonate monomers (WO1997/35869). Unnatural nucleic acids can include 5′-modified methylenephosphonate monomers (EP614907 and EP629633). Unnatural nucleic acids can include analogs of 5′ or 6′-phosphonate ribonucleosides comprising a hydroxyl group at the 5′ and/or 6′-position (Chen et al., Phosphorus, Sulfur and Silicon, 2002, 777, 1783-1786; Jung et al., Bioorg. Med. Chem., 2000, 8, 2501-2509; Gallier et al., Eur. J. Org. Chem., 2007, 925-933; and Hampton et al., J. Med. Chem., 1976, 19(8), 1029-1033). Unnatural nucleic acids can include 5′-phosphonate deoxyribonucleoside monomers and dimers having a 5′-phosphate group (Nawrot et al., Oligonucleotides, 2006, 16(1), 68-82). Unnatural nucleic acids can include nucleosides having a 6′-phosphonate group wherein the 5′ or/and 6′-position is unsubstituted or substituted with a thio-tert-butyl group (SC(CH₃)₃) (and analogs thereof); a methyleneamino group (CH₂NH₂) (and analogs thereof) or a cyano group (CN) (and analogs thereof) (Fairhurst et al., Synlett, 2001, 4, 467-472; Kappler et al., J. Med. Chem., 1986, 29, 1030-1038; Kappler et al., J. Med. Chem., 1982, 25, 1179-1184; Vrudhula et al., J. Med. Chem., 1987, 30, 888-894; Hampton et al., J. Med. Chem., 1976, 19, 1371-1377; Geze et al., J. Am. Chem. Soc, 1983, 105(26), 7638-7640; and Hampton et al., J. Am. Chem. Soc, 1973, 95(13), 4404-4414). The disclosure of each reference listed in this paragraph is incorporated herein by reference.

In some embodiments, unnatural nucleic acids also include modifications of the sugar moiety. In some cases, nucleic acids contain one or more nucleosides wherein the sugar group has been modified. Such sugar modified nucleosides may impart enhanced nuclease stability, increased binding affinity, or some other beneficial biological property. In certain embodiments, nucleic acids comprise a chemically modified ribofuranose ring moiety. Examples of chemically modified ribofuranose rings include, without limitation, addition of substituent groups (including 5′ and/or 2′ substituent groups; bridging of two ring atoms to form bicyclic nucleic acids (BNA); replacement of the ribosyl ring oxygen atom with S, N(R), or C(R₁)(R₂) (R═H, C₁-C₁₂ alkyl or a protecting group); and combinations thereof. Examples of chemically modified sugars can be found in WO2008/101157, US2005/0130923, and WO2007/134181, the disclosure of each of which is incorporated herein by reference.

In some instances, a modified nucleic acid comprises modified sugars or sugar analogs. Thus, in addition to ribose and deoxyribose, the sugar moiety can be pentose, deoxypentose, hexose, deoxyhexose, glucose, arabinose, xylose, lyxose, or a sugar “analog” cyclopentyl group. The sugar can be in a pyranosyl or furanosyl form. The sugar moiety may be the furanoside of ribose, deoxyribose, arabinose or 2′-O-alkylribose, and the sugar can be attached to the respective heterocyclic bases either in [alpha] or [beta] anomeric configuration. Sugar modifications include, but are not limited to, 2′-alkoxy-RNA analogs, 2′-amino-RNA analogs, 2′-fluoro-DNA, and 2′-alkoxy- or amino-RNA/DNA chimeras. For example, a sugar modification may include 2′-O-methyl-uridine or 2′-O-methyl-cytidine. Sugar modifications include 2′-O-alkyl-substituted deoxyribonucleosides and 2′-O-ethyleneglycol like ribonucleosides. The preparation of these sugars or sugar analogs and the respective “nucleosides” wherein such sugars or analogs are attached to a heterocyclic base (nucleic acid base) is known. Sugar modifications may also be made and combined with other modifications.

Modifications to the sugar moiety include natural modifications of the ribose and deoxy ribose as well as unnatural modifications. Sugar modifications include, but are not limited to, the following modifications at the 2′ position: OH; F; O—, S-, or N-alkyl; O—, S-, or N-alkenyl; O—, S- or N-alkynyl; or O-alkyl-O-alkyl, wherein the alkyl, alkenyl and alkynyl may be substituted or unsubstituted C₁ to C₁₀, alkyl or C₂ to C₁₀ alkenyl and alkynyl. 2′ sugar modifications also include but are not limited to —O[(CH₂)ₙO]m CH₃, —O(CH₂)ₙOCH₃, —O(CH₂)ₙNH₂, —O(CH₂)ₙCH₃, —O(CH₂)ₙONH₂, and —O(CH₂)ₙON[(CH₂)ₙ CH₃)]2, where n and m are from 1 to about 10.

Other modifications at the 2′ position include but are not limited to: C₁ to C₁₀ lower alkyl, substituted lower alkyl, alkaryl, aralkyl, O-alkaryl, O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of the 5′ terminal nucleotide. Modified sugars also include those that contain modifications at the bridging ring oxygen, such as CH₂ and S. Nucleotide sugar analogs may also have sugar mimetics such as cyclobutyl moieties in place of the pentofuranosyl sugar. There are numerous United States patents that teach the preparation of such modified sugar structures and which detail and describe a range of base modifications, such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; and 5,700,920, the disclosure of each of which is incorporated herein by reference.

Examples of nucleic acids having modified sugar moieties include, without limitation, nucleic acids comprising 5′-vinyl, 5′-methyl (R or S), 4′-S, 2′-F, 2′-OCH₃, and 2′-O(CH₂)₂OCH₃ substituent groups. The substituent at the 2′ position can also be selected from allyl, amino, azido, thio, O-allyl, O—(C₁-C₁₀ alkyl), OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl.

In certain embodiments, nucleic acids described herein include one or more bicyclic nucleic acids. In certain such embodiments, the bicyclic nucleic acid comprises a bridge between the 4′ and the 2′ ribosyl ring atoms. In certain embodiments, nucleic acids provided herein include one or more bicyclic nucleic acids wherein the bridge comprises a 4′ to 2′ bicyclic nucleic acid. Examples of such 4′ to 2′ bicyclic nucleic acids include, but are not limited to, one of the formulae: 4′-(CH₂)—O-2′ (LNA); 4′-(CH₂)—S-2′; 4′-(CH₂)₂—O-2′ (ENA); 4′-CH(CH₃)—O-2′ and 4′-CH(CH₂OCH₃)—O-2′, and analogs thereof (see, U.S. Pat. No. 7,399,845); 4′-C(CH₃)(CH₃)—O-2′ and analogs thereof, (see WO2009/006478, WO2008/150729, US2004/0171570, U.S. Pat. No. 7,427,672, Chattopadhyaya et al., J. Org. Chem., 209, 74, 118-134, and WO2008/154401). Also see, for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129(26) 8362-8379; Elayadi et al., Curr. Opinion Invens. Drugs, 2001, 2, 558-561; Braasch et al., Chem. Biol, 2001, 8, 1-7; Oram et al., Curr. Opinion Mol. Ther., 2001, 3, 239-243; U.S. Pat. Nos. 4,849,513; 5,015,733; 5,118,800; 5,118,802; 7,053,207; 6,268,490; 6,770,748; 6,794,499; 7,034,133; 6,525,191; 6,670,461; and 7,399,845; International Publication Nos. WO2004/106356, WO1994/14226, WO2005/021570, WO2007/090071, and WO2007/134181; U.S. Patent Publication Nos. US2004/0171570, US2007/0287831, and US2008/0039618; U.S. Provisional Application Nos. 60/989,574, 61/026,995, 61/026,998, 61/056,564, 61/086,231, 61/097,787, and 61/099,844; and International Applications Nos. PCT/US2008/064591, PCT US2008/066154, PCT US2008/068922, and PCT/DK98/00393. The disclosure of each reference listed in this paragraph is incorporated herein by reference.

In certain embodiments, nucleic acids comprise linked nucleic acids. Nucleic acids can be linked together using any inter nucleic acid linkage. The two main classes of inter nucleic acid linking groups are defined by the presence or absence of a phosphorus atom. Representative phosphorus containing inter nucleic acid linkages include, but are not limited to, phosphodiesters, phosphotriesters, methylphosphonates, phosphoramidate, and phosphorothioates (P═S). Representative non-phosphorus containing inter nucleic acid linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N*-dimethylhydrazine (—CH₂—N(CH₃)—N(CH₃)). In certain embodiments, inter nucleic acids linkages having a chiral atom can be prepared as a racemic mixture, as separate enantiomers, e.g., alkylphosphonates and phosphorothioates. Unnatural nucleic acids can contain a single modification. Unnatural nucleic acids can contain multiple modifications within one of the moieties or between different moieties.

Backbone phosphate modifications to nucleic acid include, but are not limited to, methyl phosphonate, phosphorothioate, phosphoramidate (bridging or non-bridging), phosphotriester, phosphorodithioate, phosphodithioate, and boranophosphate, and may be used in any combination. Other non-phosphate linkages may also be used.

In some embodiments, backbone modifications (e.g., methylphosphonate, phosphorothioate, phosphoroamidate and phosphorodithioate internucleotide linkages) can confer immunomodulatory activity on the modified nucleic acid and/or enhance their stability in vivo.

In some instances, a phosphorous derivative (or modified phosphate group) is attached to the sugar or sugar analog moiety in and can be a monophosphate, diphosphate, triphosphate, alkylphosphonate, phosphorothioate, phosphorodithioate, phosphoramidate or the like. Exemplary polynucleotides containing modified phosphate linkages or non-phosphate linkages can be found in Peyrottes et al., 1996, Nucleic Acids Res. 24: 1841-1848; Chaturvedi et al., 1996, Nucleic Acids Res. 24:2318-2323; Schultz et al., (1996) Nucleic Acids Res. 24:2966-2973; Matteucci, 1997, “Oligonucleotide Analogs: an Overview” in Oligonucleotides as Therapeutic Agents, (Chadwick and Cardew, ed.) John Wiley and Sons, New York, NY; Zon, 1993, “Oligonucleoside Phosphorothioates” in Protocols for Oligonucleotides and Analogs, Synthesis and Properties, Humana Press, pp. 165-190; Miller et al., 1971, JACS 93:6657-6665; Jager et al., 1988, Biochem. 27:7247-7246; Nelson et al., 1997, JOC 62:7278-7287; U.S. Pat. No. 5,453,496; and Micklefield, 2001, Curr. Med. Chem. 8: 1157-1179; the disclosure of each of which is incorporated herein by reference.

In some cases, backbone modification comprises replacing the phosphodiester linkage with an alternative moiety such as an anionic, neutral or cationic group. Examples of such modifications include: anionic internucleoside linkage; N₃′ to P5′ phosphoramidate modification; boranophosphate DNA; prooligonucleotides; neutral internucleoside linkages such as methylphosphonates; amide linked DNA; methylene(methylimino) linkages; formacetal and thioformacetal linkages; backbones containing sulfonyl groups; morpholino oligos; peptide nucleic acids (PNA); and positively charged deoxyribonucleic guanidine (DNG) oligos (Micklefield, 2001, Current Medicinal Chemistry 8: 1157-1179, the disclosure of which is incorporated herein by reference). A modified nucleic acid may comprise a chimeric or mixed backbone comprising one or more modifications, e.g. a combination of phosphate linkages such as a combination of phosphodiester and phosphorothioate linkages.

Substitutes for the phosphate include, for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439. It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. See also Nielsen et al., Science, 1991, 254, 1497-1500. It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. KY. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EM50J, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1-di-O-hexadecyl-rac-glycero-S—H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochem. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937). Numerous United States patents teach the preparation of such conjugates and include, but are not limited to U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941. The disclosure of each reference listed in this paragraph is incorporated herein by reference.

In some cases, the unnatural nucleic acids further form unnatural base pairs. Exemplary unnatural nucleotides capable of forming an unnatural DNA or RNA base pair (UBP) under conditions in vivo includes, but is not limited to, TAT1, dTAT1, 5FM, d5FM, TPT3, dTPT3, 5SICS, d5SICS, NaM, dNaM, CNMO, dCNMO, and combinations thereof. In some embodiments, unnatural nucleotides include:

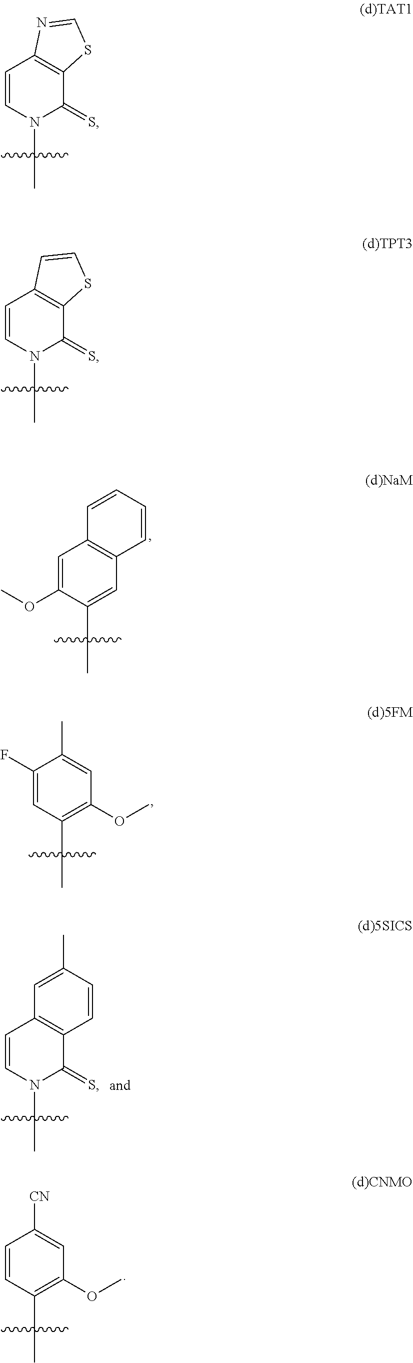 [SMILES: *n1ccc2ncsc2c1=S ; *n1ccc2ccsc2c1=S ; *c1cc2ccccc2cc1OC ; *c1cc(F)c(C)cc1OC ; *n1ccc2cc(C)ccc2c1=S ; *c1ccc(C#N)cc1OC]

Exemplary unnatural base pairs include: (d) TPT3-(d) NaM; (d)5SICS-(d) NaM; (d) CNMO-(d) TAT1; (d)NaM-(d)TAT1; (d)CNMO-(d)TPT3; and (d)5FM-(d)TAT1.

Other examples of unnatural nucleotides capable of forming unnatural UBPs that may be used to prepare the IL-2 conjugates disclosed herein may be found in Dien et al., J Am Chem Soc., 2018, 140:16115-16123; Feldman et al., J Am Chem Soc, 2017, 139:11427-11433; Ledbetter et al., J Am Chem Soc., 2018, 140:758-765; Dhami et al., Nucleic Acids Res. 2014, 42:10235-10244; Malyshev et al., Nature, 2014, 509:385-388; Betz et al., J Am Chem Soc., 2013, 135:18637-18643; Lavergne et al., J Am Chem Soc. 2013, 135:5408-5419; and Malyshev et al. Proc Natl Acad Sci USA, 2012, 109:12005-12010; the disclosure of each of which is incorporated herein by reference. In some embodiments, unnatural nucleotides include:

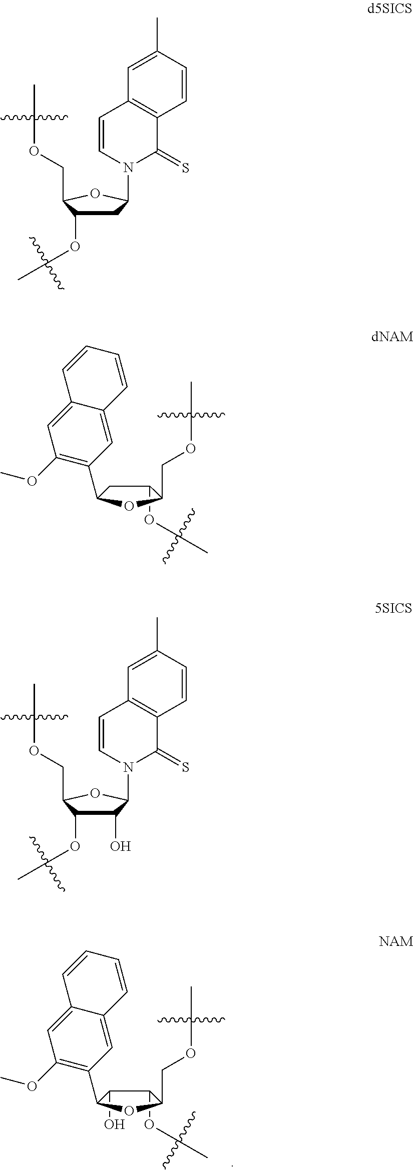

In some embodiments, the unnatural nucleotides that may be used to prepare the IL-2 conjugates disclosed herein may be derived from a compound of the formula

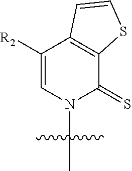

wherein R₂ is selected from the group consisting of hydrogen, alkyl, alkenyl, alkynyl, methoxy, methanethiol, methaneseleno, halogen, cyano, and azido; and
the wavy line indicates a bond to a ribosyl or 2′-deoxyribosyl, wherein the 5′-hydroxy group of the ribosyl or 2′-deoxyribosyl moiety is in free form, is connected to a monophosphate, diphosphate, triphosphate, α-thiotriphosphate, β-thiotriphosphate, or γ-thiotriphosphate group, or is included in an RNA or a DNA or in an RNA analog or a DNA analog.

In some embodiments, the unnatural nucleotides that may be used to prepare the IL-2 conjugates disclosed herein may be derived from a compound of the Formula

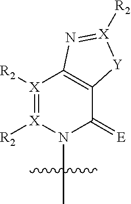

wherein:
each X is independently carbon or nitrogen;
R₂ is absent when X is nitrogen, and is present when X is carbon and is independently hydrogen, alkyl, alkenyl, alkynyl, methoxy, methanethiol, methaneseleno, halogen, cyano, or azide;
Y is sulfur, oxygen, selenium, or secondary amine;
E is oxygen, sulfur, or selenium; and
the wavy line indicates a point of bonding to a ribosyl, deoxyribosyl, or dideoxyribosyl moiety or an analog thereof, wherein the ribosyl, deoxyribosyl, or dideoxyribosyl moiety or analog thereof is in free form, is connected to a mono-phosphate, diphosphate, triphosphate, α-thiotriphosphate, β-thiotriphosphate, or γ-thiotriphosphate group, or is included in an RNA or a DNA or in an RNA analog or a DNA analog.

In some embodiments, each X is carbon. In some embodiments, at least one X is carbon. In some embodiments, one X is carbon. In some embodiments, at least two X are carbon. In some embodiments, two X are carbon. In some embodiments, at least one X is nitrogen. In some embodiments, one X is nitrogen. In some embodiments, at least two X are nitrogen. In some embodiments, two X are nitrogen.

In some embodiments, Y is sulfur. In some embodiments, Y is oxygen. In some embodiments, Y is selenium. In some embodiments, Y is a secondary amine.

In some embodiments, E is sulfur. In some embodiments, E is oxygen. In some embodiments, E is selenium.

In some embodiments, R₂ is present when X is carbon. In some embodiments, R₂ is absent when X is nitrogen. In some embodiments, each R₂, where present, is hydrogen. In some embodiments, R₂ is alkyl, such as methyl, ethyl, or propyl. In some embodiments, R₂ is alkenyl, such as —CH₂═CH₂. In some embodiments, R₂ is alkynyl, such as ethynyl. In some embodiments, R₂ is methoxy. In some embodiments, R₂ is methanethiol. In some embodiments, R₂ is methaneseleno. In some embodiments, R₂ is halogen, such as chloro, bromo, or fluoro. In some embodiments, R₂ is cyano. In some embodiments, R₂ is azide.

In some embodiments, E is sulfur, Y is sulfur, and each X is independently carbon or nitrogen. In some embodiments, E is sulfur, Y is sulfur, and each X is carbon.

In some embodiments, the unnatural nucleotides that may be used to prepare the IL-2 conjugates disclosed herein may be derived from

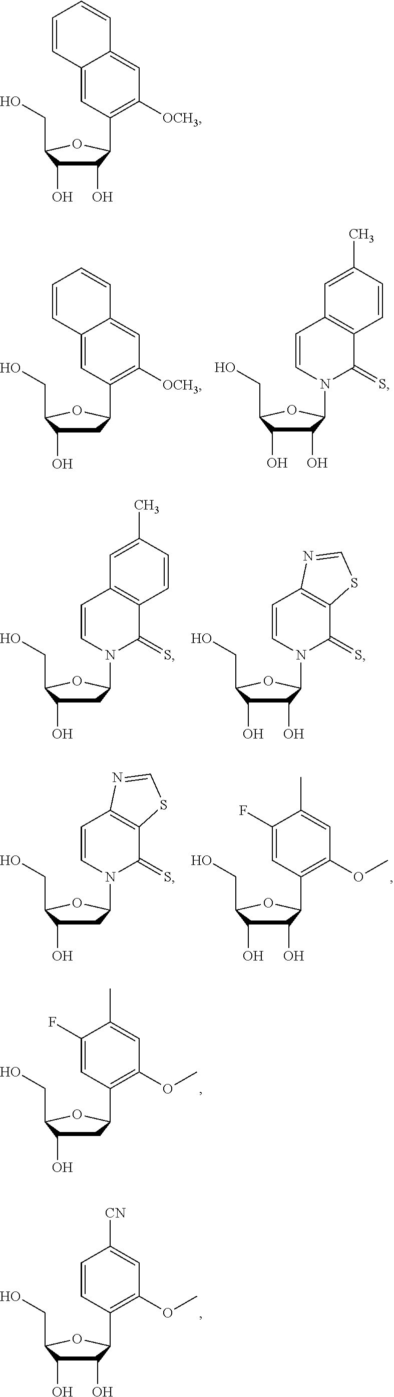

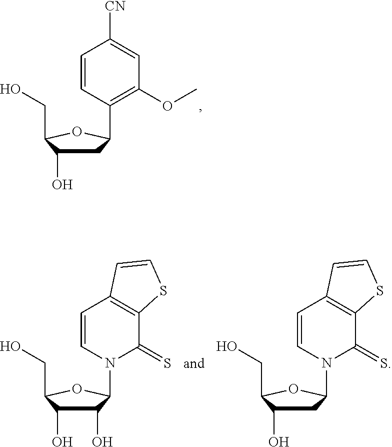

In some embodiments, the unnatural nucleotides that may be used to prepare the IL-2 conjugates disclosed herein include

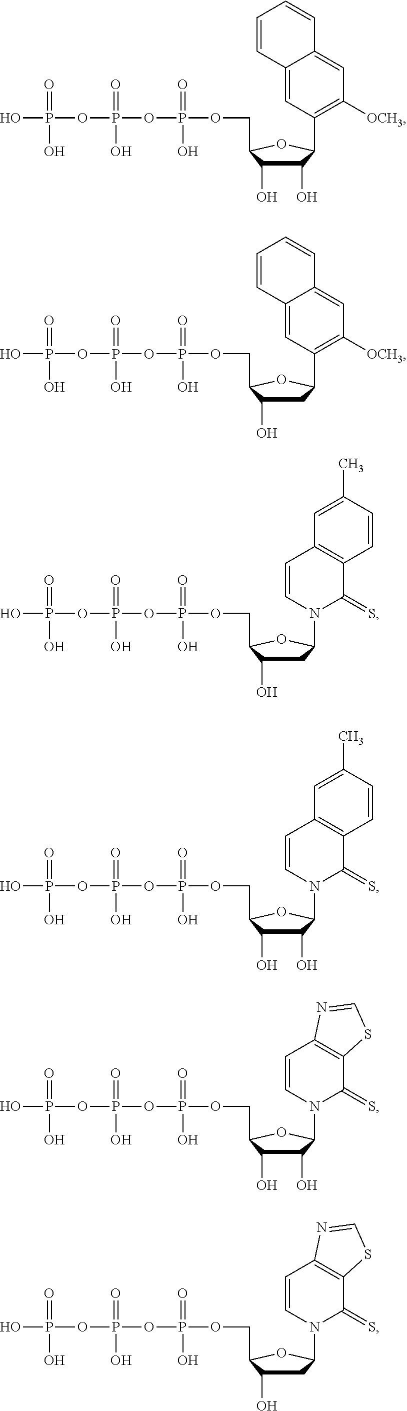

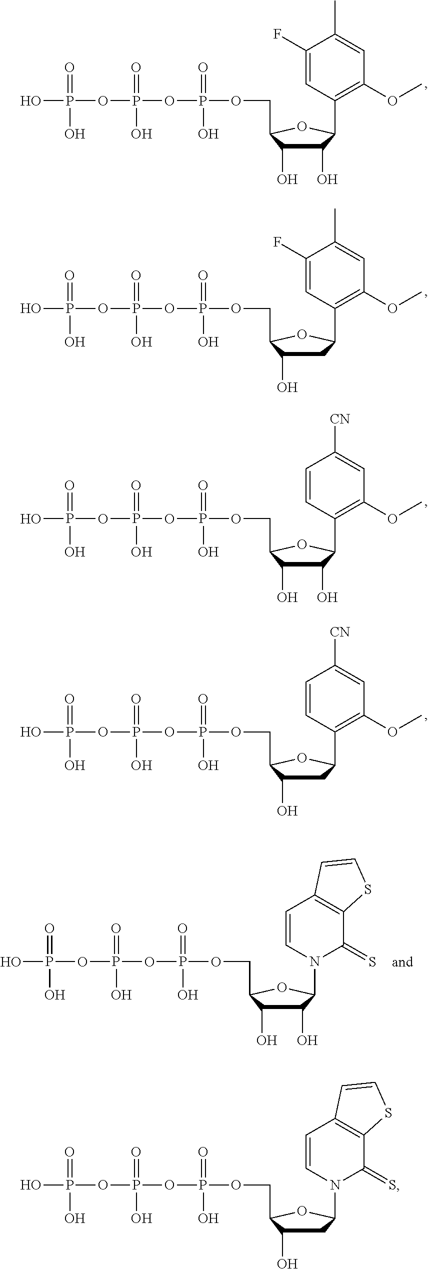

or salts thereof.

In some embodiments, an unnatural base pair generate an unnatural amino acid described in Dumas et al., “Designing logical codon reassignment—Expanding the chemistry in biology,” Chemical Science, 6: 50-69 (2015), the disclosure of which is incorporated herein by reference.

In some embodiments, the unnatural amino acid is incorporated into the cytokine (e.g., the IL polypeptide) by a synthetic codon comprising an unnatural nucleic acid. In some instances, the unnatural amino acid is incorporated into the cytokine by an orthogonal, modified synthetase/tRNA pair. Such orthogonal pairs comprise an unnatural synthetase that is capable of charging the unnatural tRNA with the unnatural amino acid, while minimizing charging of a) other endogenous amino acids onto the unnatural tRNA and b) unnatural amino acids onto other endogenous tRNAs. Such orthogonal pairs comprise tRNAs that are capable of being charged by the unnatural synthetase, while avoiding being charged with a) other endogenous amino acids by endogenous synthetases. In some embodiments, such pairs are identified from various organisms, such as bacteria, yeast, Archaea, or human sources. In some embodiments, an orthogonal synthetase/tRNA pair comprises components from a single organism. In some embodiments, an orthogonal synthetase/tRNA pair comprises components from two different organisms. In some embodiments, an orthogonal synthetase/tRNA pair comprising components that prior to modification, promote translation of two different amino acids. In some embodiments, an orthogonal synthetase is a modified alanine synthetase. In some embodiments, an orthogonal synthetase is a modified arginine synthetase. In some embodiments, an orthogonal synthetase is a modified asparagine synthetase. In some embodiments, an orthogonal synthetase is a modified aspartic acid synthetase. In some embodiments, an orthogonal synthetase is a modified cysteine synthetase. In some embodiments, an orthogonal synthetase is a modified glutamine synthetase. In some embodiments, an orthogonal synthetase is a modified glutamic acid synthetase. In some embodiments, an orthogonal synthetase is a modified alanine glycine. In some embodiments, an orthogonal synthetase is a modified histidine synthetase. In some embodiments, an orthogonal synthetase is a modified leucine synthetase. In some embodiments, an orthogonal synthetase is a modified isoleucine synthetase. In some embodiments, an orthogonal synthetase is a modified lysine synthetase. In some embodiments, an orthogonal synthetase is a modified methionine synthetase. In some embodiments, an orthogonal synthetase is a modified phenylalanine synthetase. In some embodiments, an orthogonal synthetase is a modified proline synthetase. In some embodiments, an orthogonal synthetase is a modified serine synthetase. In some embodiments, an orthogonal synthetase is a modified threonine synthetase. In some embodiments, an orthogonal synthetase is a modified tryptophan synthetase. In some embodiments, an orthogonal synthetase is a modified tyrosine synthetase. In some embodiments, an orthogonal synthetase is a modified valine synthetase. In some embodiments, an orthogonal synthetase is a modified phosphoserine synthetase. In some embodiments, an orthogonal tRNA is a modified alanine tRNA. In some embodiments, an orthogonal tRNA is a modified arginine tRNA. In some embodiments, an orthogonal tRNA is a modified asparagine tRNA. In some embodiments, an orthogonal tRNA is a modified aspartic acid tRNA. In some embodiments, an orthogonal tRNA is a modified cysteine tRNA. In some embodiments, an orthogonal tRNA is a modified glutamine tRNA. In some embodiments, an orthogonal tRNA is a modified glutamic acid tRNA. In some embodiments, an orthogonal tRNA is a modified alanine glycine. In some embodiments, an orthogonal tRNA is a modified histidine tRNA. In some embodiments, an orthogonal tRNA is a modified leucine tRNA. In some embodiments, an orthogonal tRNA is a modified isoleucine tRNA. In some embodiments, an orthogonal tRNA is a modified lysine tRNA. In some embodiments, an orthogonal tRNA is a modified methionine tRNA. In some embodiments, an orthogonal tRNA is a modified phenylalanine tRNA. In some embodiments, an orthogonal tRNA is a modified proline tRNA. In some embodiments, an orthogonal tRNA is a modified serine tRNA. In some embodiments, an orthogonal tRNA is a modified threonine tRNA. In some embodiments, an orthogonal tRNA is a modified tryptophan tRNA. In some embodiments, an orthogonal tRNA is a modified tyrosine tRNA. In some embodiments, an orthogonal tRNA is a modified valine tRNA. In some embodiments, an orthogonal tRNA is a modified phosphoserine tRNA.

In some embodiments, the unnatural amino acid is incorporated into the cytokine (e.g., the IL polypeptide) by an aminoacyl (aaRS or RS)-tRNA synthetase-tRNA pair. Exemplary aaRS-tRNA pairs include, but are not limited to, Methanococcus jannaschii (Mj-Tyr) aaRS/tRNA pairs, E. coli TyrRS (Ec-Tyr)/B. stearothermophilus tRNA_{cuA} pairs, E. coli LeuRS (Ec-Leu)/B. stearothermophilus tRNA_{cuA} pairs, and pyrrolysyl-tRNA pairs. In some instances, the unnatural amino acid is incorporated into the cytokine (e.g., the IL polypeptide) by a Mj-TyrRS/tRNA pair. Exemplary UAAs that can be incorporated by a Mj-TyrRS/tRNA pair include, but are not limited to, para-substituted phenylalanine derivatives such as p-aminophenylalanine andp-methoyphenylalanine; meta-substituted tyrosine derivatives such as 3-aminotyrosine, 3-nitrotyrosine, 3,4-dihydroxyphenylalanine, and 3-iodotyrosine; phenylselenocysteine; p-boronophenylalanine; and o-nitrobenzyltyrosine.

In some instances, the unnatural amino acid is incorporated into the cytokine (e.g., the IL polypeptide) by a Ec-Tyr/tRNA_{cuA} or a Ec-Leu/tRNA_{cuA} pair. Exemplary UAAs that can be incorporated by a Ec-Tyr/tRNA_{cuA} or a Ec-Leu/tRNA_{cuA} pair include, but are not limited to, phenylalanine derivatives containing benzophenone, ketone, iodide, or azide substituents; O-propargyltyrosine; α-aminocaprylic acid, O-methyl tyrosine, O-nitrobenzyl cysteine; and 3-(naphthalene-2-ylamino)-2-amino-propanoic acid.

In some instances, the unnatural amino acid is incorporated into the cytokine (e.g., the IL polypeptide) by a pyrrolysyl-tRNA pair. In some cases, the PylRS is obtained from an archaebacterial, e.g., from a methanogenic archaebacterial. In some cases, the PylRS is obtained from Methanosarcina barkeri, Methanosarcina mazei, or Methanosarcina acetivorans. Exemplary UAAs that can be incorporated by a pyrrolysyl-tRNA pair include, but are not limited to, amide and carbamate substituted lysines such as 2-amino-6-((R)-tetrahydrofuran-2-carboxamido)hexanoic acid, N-ε-D-prolyl-L-lysine, and N-ε-cyclopentyloxycarbonyl-_{L}-lysine; N-ε-Acryloyl-L-lysine; N-ε-[(1-(6-nitrobenzo[d][1,3]dioxol-5-yl)ethoxy)carbonyl]-_{L}-lysine; and N-ε-(1-methylcyclopro-2-enecarboxamido)lysine. In some embodiments, the IL-2 conjugates disclosed herein may be prepared by use of M. mazei tRNA which is selectively charged with a non-natural amino acid such as N6-((2-azidoethoxy)-carbonyl)-_{L}-lysine (AzK) by the M. barkeri pyrrolysyl-tRNA synthetase (Mb PylRS). Other methods are known to those of ordinary skill in the art, such as those disclosed in Zhang et al., Nature 2017, 551(7682): 644-647, the disclosure of which is incorporated herein by reference.

In some instances, an unnatural amino acid is incorporated into a cytokine described herein (e.g., the IL polypeptide) by a synthetase disclosed in U.S. Pat. Nos. 9,988,619 and 9,938,516, the disclosure of each of which is incorporated herein by reference.

The host cell into which the constructs or vectors disclosed herein are introduced is cultured or maintained in a suitable medium such that the tRNA, the tRNA synthetase and the protein of interest are produced. The medium also comprises the unnatural amino acid(s) such that the protein of interest incorporates the unnatural amino acid(s). In some embodiments, a nucleoside triphosphate transporter (NTT) from bacteria, plant, or algae is also present in the host cell. In some embodiments, the IL-2 conjugates disclosed herein are prepared by use of a host cell that expresses a NTT. In some embodiments, the nucleotide nucleoside triphosphate transporter used in the host cell may be selected from TpNTT1, TpNTT2, TpNTT3, TpNTT4, TpNTT5, TpNTT6, TpNTT7, TpNTT8 (T. pseudonana), PtNTT1, PtNTT2, PtNTT3, PtNTT4, PtNTT5, PtNTT6 (P. tricornutum), GsNTT (Galdieria sulphuraria), AtNTT1, AtNTT2 (Arabidopsis thaliana), CtNTT1, CtNTT2 (Chlamydia trachomatis), PamNTTT, PamNTT2 (Protochlamydia amoebophila), CcNTT (Caedibacter caryophilus), RpNTTT (Rickettsia prowazekii). In some embodiments, the NTT is selected from PtNTT1, PtNTT2, PtNTT3, PtNTT4, PtNTT5, and PtNTT6. In some embodiments, the NTT is PtNTT1. In some embodiments, the NTT is PtNTT2. In some embodiments, the NTT is PtNTT3. In some embodiments, the NTT is PtNTT4. In some embodiments, the NTT is PtNTT5. In some embodiments, the NTT is PtNTT6. Other NTTs that may be used are disclosed in Zhang et al., Nature 2017, 551(7682): 644-647; Malyshev et al. Nature 2014 (509(7500), 385-388; and Zhang et al. Proc Natl Acad Sci USA, 2017, 114:1317-1322.

The orthogonal tRNA synthetase/tRNA pair charges a tRNA with an unnatural amino acid and incorporates the unnatural amino acid into the polypeptide chain in response to the codon. Exemplary aaRS-tRNA pairs include, but are not limited to, Methanococcus jannaschii (Mj-Tyr) aaRS/tRNA pairs, E. coli TyrRS (Ec-Tyr)/B. stearothermophilus tRNA_{cuA} pairs, E. coli LeuRS (Ec-Leu)/B. stearothermophilus tRNA_{cuA} pairs, and pyrrolysyl-tRNA pairs. Other aaRS-tRNA pairs that may be used according to the present disclosure include those derived from M. mazei those described in Feldman et al., J Am Chem Soc., 2018 140:1447-1454; and Zhang et al. Proc Natl Acad Sci USA, 2017, 114:1317-1322; the disclosure of each of which is incorporated herein by reference.

In some embodiments are provided methods of preparing the IL-2 conjugates disclosed herein in a cellular system that expresses a NTT and a tRNA synthetase. In some embodiments described herein, the NTT is selected from PtNTT1, PtNTT2, PtNTT3, PtNTT4, PtNTT5, and PtNTT6, and the tRNA synthetase is selected from Methanococcus jannaschii, E. coli TyrRS (Ec-Tyr)/B. stearothermophilus, and M. mazei. In some embodiments, the NTT is PtNTT1 and the tRNA synthetase is derived from Methanococcus jannaschii, E. coli TyrRS (Ec-Tyr)/B. stearothermophilus, or M. mazei. In some embodiments, the NTT is PtNTT2 and the tRNA synthetase is derived from Methanococcus jannaschii, E. coli TyrRS (Ec-Tyr)/B. stearothermophilus, or M. mazei. In some embodiments, the NTT is PtNTT3 and the tRNA synthetase is derived from Methanococcus jannaschii, E. coli TyrRS (Ec-Tyr)/B. stearothermophilus, or M. mazei. In some embodiments, the NTT is PtNTT3 and the tRNA synthetase is derived from Methanococcus jannaschii, E. coli TyrRS (Ec-Tyr)/B. stearothermophilus, or M. mazei. In some embodiments, the NTT is PtNTT4 and the tRNA synthetase is derived from Methanococcus jannaschii, E. coli TyrRS (Ec-Tyr)/B. stearothermophilus, or M. mazei. In some embodiments, the NTT is PtNTT5 and the tRNA synthetase is derived from Methanococcus jannaschii, E. coli TyrRS (Ec-Tyr)/B. stearothermophilus, or M. mazei. In some embodiments, the NTT is PtNTT6 and the tRNA synthetase is derived from Methanococcus jannaschii, E. coli TyrRS (Ec-Tyr)/B. stearothermophilus, or M. mazei.

In some embodiments, the IL-2 conjugates disclosed herein may be prepared in a cell, such as E. coli, comprising (a) nucleotide triphosphate transporter PtNTT2 (including a truncated variant in which the first 65 amino acid residues of the full-length protein are deleted), (b) a plasmid comprising a double-stranded oligonucleotide that encodes an IL-2 variant having a desired amino acid sequence and that contains a unnatural base pair comprising a first unnatural nucleotide and a second unnatural nucleotide to provide a codon at the desired position at which an unnatural amino acid, such as N6-((2-azidoethoxy)-carbonyl)-_{L}-lysine (AzK), will be incorporated, (c) a plasmid encoding a tRNA derived from M. mazei and which comprises an unnatural nucleotide to provide a recognized anticodon (to the codon of the IL-2 variant) in place of its native sequence, and (d) a plasmid encoding a M. barkeri derived pyrrolysyl-tRNA synthetase (Mb PylRS), which may be the same plasmid that encodes the tRNA or a different plasmid. In some embodiments, the cell is further supplemented with deoxyribo triphosphates comprising one or more unnatural bases. In some embodiments, the cell is further supplemented with ribo triphosphates comprising one or more unnatural bases. In some embodiments, the cells is further supplemented with one or more unnatural amino acids, such as N6-((2-azidoethoxy)-carbonyl)-L-lysine (AzK). In some embodiments, the double-stranded oligonucleotide that encodes the amino acid sequence of the desired IL-2 variant contains a codon AXC at position 64 of the sequence that encodes the protein having SEQ ID NO: 1, wherein X is an unnatural nucleotide. In some embodiments, the cell further comprises a plasmid, which may be the protein expression plasmid or another plasmid, that encodes an orthogonal tRNA gene from M. mazei that comprises an AXC-matching anticodon GYT in place of its native sequence, wherein Y is an unnatural nucleotide that is complementary and may be the same or different as the unnatural nucleotide in the codon. In some embodiments, the unnatural nucleotide in the codon is different than and complimentary to the unnatural nucleotide in the anti-codon. In some embodiments, the unnatural nucleotide in the codon is the same as the unnatural nucleotide in the anti-codon. In some embodiments, the first and second unnatural nucleotides comprising the unnatural base pair in the double-stranded oligonucleotide may be derived from

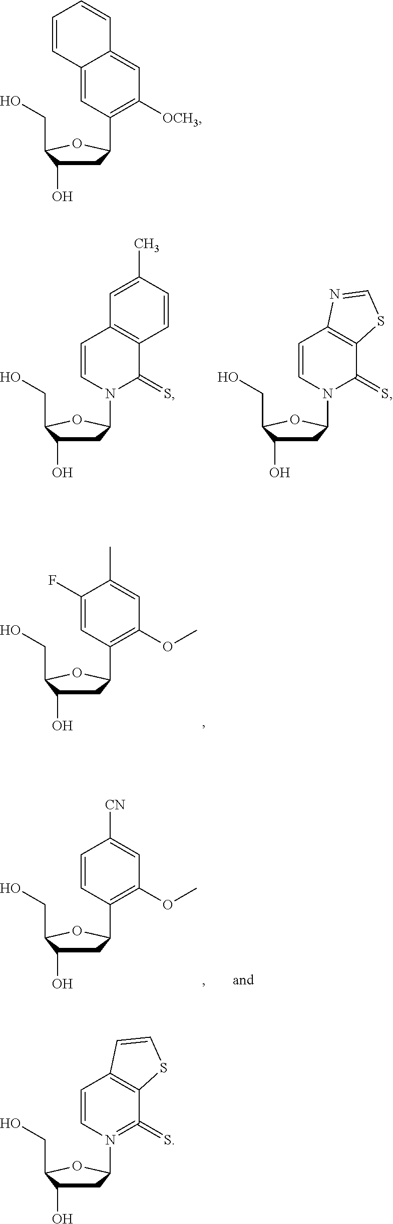

In some embodiments, the first and second unnatural nucleotides comprising the unnatural base pair in the double-stranded oligonucleotide may be derived from

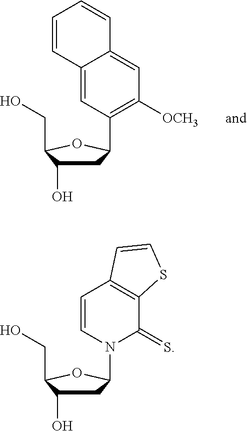

In some embodiments, the triphosphates of the first and second unnatural nucleotides include,

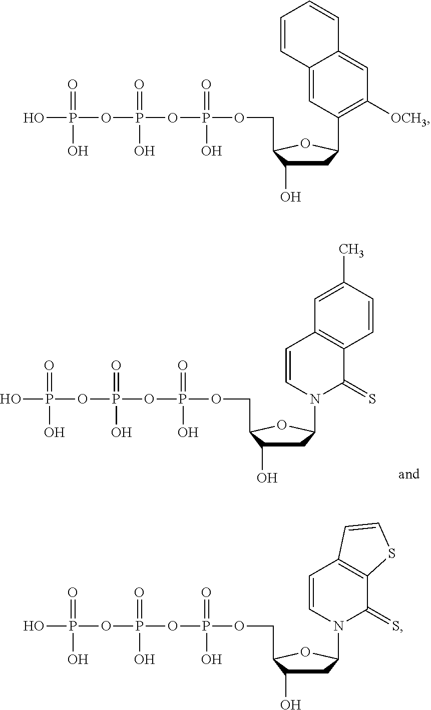

or salts thereof. In some embodiments, the triphosphates of the first and second unnatural nucleotides include,

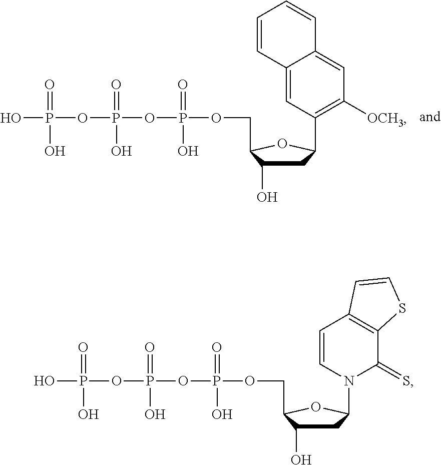

or salts thereof. In some embodiments, the mRNA derived the double-stranded oligonucleotide comprising a first unnatural nucleotide and a second unnatural nucleotide may comprise a codon comprising an unnatural nucleotide derived from

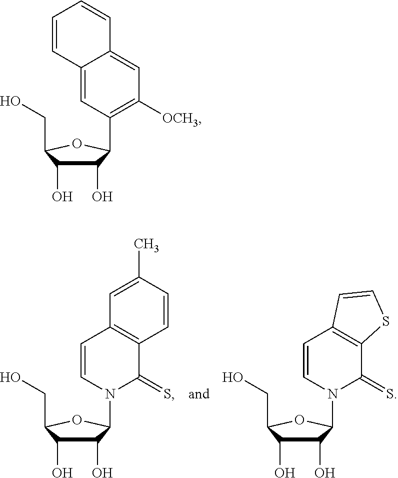

In some embodiments, the M. mazei tRNA may comprise an anti-codon comprising an unnatural nucleotide that recognizes the codon comprising the unnatural nucleotide of the mRNA. The anti-codon in the M. mazei tRNA may comprise an unnatural nucleotide derived from

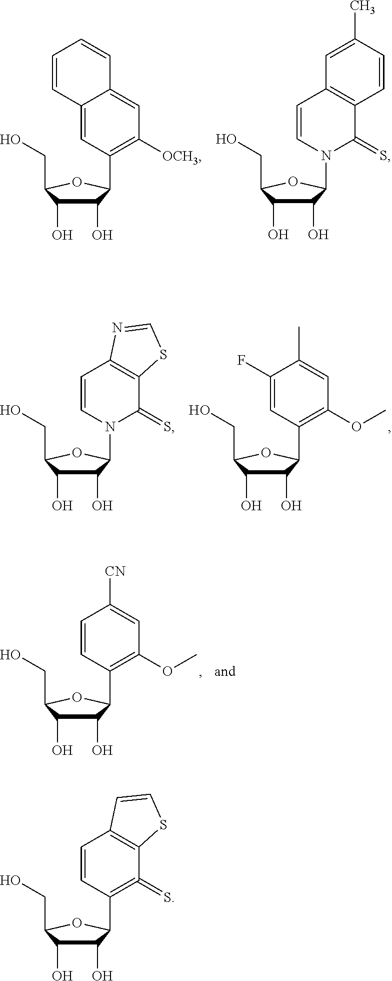

In some embodiments, the mRNA comprises an unnatural nucleotide derived from

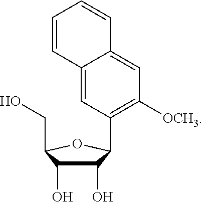

In some embodiments, the mRNA comprises an unnatural nucleotide derived from

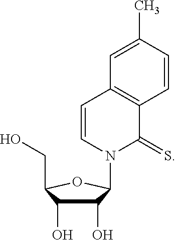

In some embodiments, the mRNA comprises an unnatural nucleotide derived from

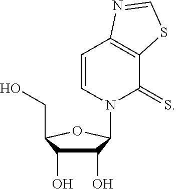

In some embodiments, the mRNA comprises an unnatural nucleotide derived from

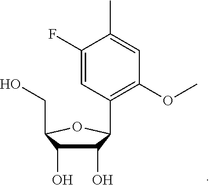

In some embodiments, the mRNA comprises an unnatural nucleotide derived from

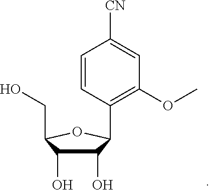

In some embodiments, the mRNA comprises an unnatural nucleotide derived from

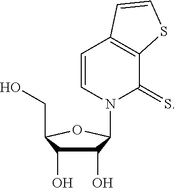

In some embodiments, the tRNA comprises an unnatural nucleotide derived from

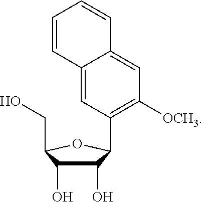

In some embodiments, the tRNA comprises an unnatural nucleotide derived from

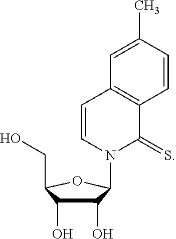

In some embodiments, the tRNA comprises an unnatural nucleotide derived from

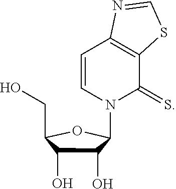

In some embodiments, the tRNA comprises an unnatural nucleotide derived from

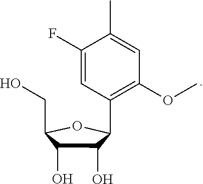

In some embodiments, the tRNA comprises an unnatural nucleotide derived from

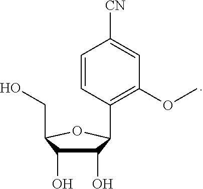

In some embodiments, the tRNA comprises an unnatural nucleotide derived from

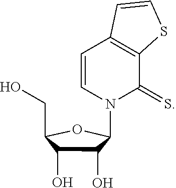

In some embodiments, the mRNA comprises an unnatural nucleotide derived from

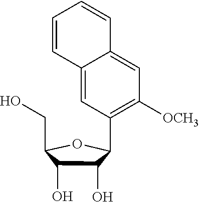

and the tRNA comprises an unnatural nucleotide derived from

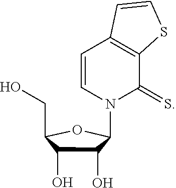

In some embodiments, the mRNA comprises an unnatural nucleotide derived from

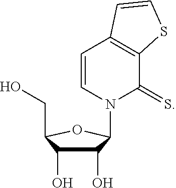

and the tRNA comprises an unnatural nucleotide derived from

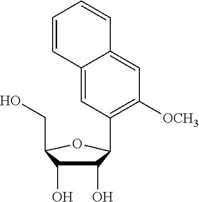

In some embodiments, the mRNA comprises an unnatural nucleotide derived from

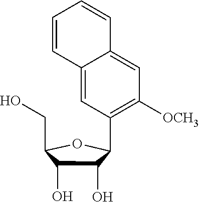

and the tRNA comprises an unnatural nucleotide derived from

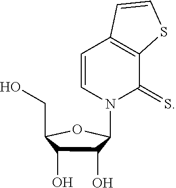

In some embodiments, the mRNA comprises an unnatural nucleotide derived from

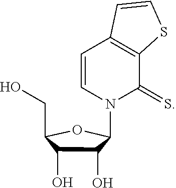

and the tRNA comprises an unnatural nucleotide derived from

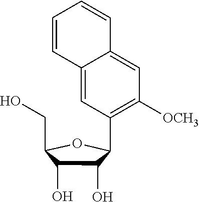

The host cell is cultured in a medium containing appropriate nutrients, and is supplemented with (a) the triphosphates of the deoxyribo nucleosides comprising one or more unnatural bases that are necessary for replication of the plasmid(s) encoding the cytokine gene harboring the codon, (b) the triphosphates of the ribo nucleosides comprising one or more unnatural bases necessary for transcription of (i) the mRNA corresponding to the coding sequence of the cytokine and containing the codon comprising one or more unnatural bases, and (ii) the tRNA containing the anticodon comprising one or more unnatural bases, and (c) the unnatural amino acid(s) to be incorporated in to the polypeptide sequence of the cytokine of interest. The host cells are then maintained under conditions which permit expression of the protein of interest.

The resulting AzK-containing protein that is expressed may be purified by methods known to those of ordinary skill in the art and may then be allowed to react with an alkyne, such as DBCO comprising a PEG chain having a desired average molecular weight as disclosed herein, under conditions known to those of ordinary skill in the art, to afford the IL-2 conjugates disclosed herein. Other methods are known to those of ordinary skill in the art, such as those disclosed in Zhang et al., Nature 2017, 551(7682): 644-647; WO 2015157555; WO 2015021432; WO 2016115168; WO 2017106767; WO 2017223528; WO 2019014262; WO 2019014267; WO 2019028419; and WO2019/028425; the disclosure of each of which is incorporated herein by reference.

The resulting protein comprising the one or more unnatural amino acids, Azk for example, that is expressed may be purified by methods known to those of ordinary skill in the art and may then be allowed to react with an alkyne, such as DBCO comprising a PEG chain having a desired average molecular weight as disclosed herein, under conditions known to those of ordinary skill in the art, to afford the IL-2 conjugates disclosed herein. Other methods are known to those of ordinary skill in the art, such as those disclosed in Zhang et al., Nature 2017, 551(7682): 644-647; WO 2015157555; WO 2015021432; WO 2016115168; WO 2017106767; WO 2017223528; WO 2019014262; WO 2019014267; WO 2019028419; and WO2019/028425; the disclosure of each of which is incorporated herein by reference.

Alternatively, an IL-2 polypeptide comprising an unnatural amino acid(s) is prepared by introducing the nucleic acid constructs described herein comprising the tRNA and aminoacyl tRNA synthetase and comprising a nucleic acid sequence of interest with one or more in-frame orthogonal (stop) codons into a host cell. The host cell is cultured in a medium containing appropriate nutrients, is supplemented with (a) the triphosphates of the deoxyribo nucleosides comprising one or more unnatural bases required for replication of the plasmid(s) encoding the cytokine gene harboring the new codon and anticodon, (b) the triphosphates of the ribo nucleosides required for transcription of the mRNA corresponding to (i) the cytokine sequence containing the codon, and (ii) the orthogonal tRNA containing the anticodon, and (c) the unnatural amino acid(s). The host cells are then maintained under conditions which permit expression of the protein of interest. The unnatural amino acid(s) is incorporated into the polypeptide chain in response to the unnatural codon. For example, one or more unnatural amino acids are incorporated into the IL-2 polypeptide. Alternatively, two or more unnatural amino acids may be incorporated into the IL-2 polypeptide at two or more sites in the protein.

Once the IL-2 polypeptide incorporating the unnatural amino acid(s) has been produced in the host cell it can be extracted therefrom by a variety of techniques known in the art, including enzymatic, chemical and/or osmotic lysis and physical disruption. The IL-2 polypeptide can be purified by standard techniques known in the art such as preparative ion exchange chromatography, hydrophobic chromatography, affinity chromatography, or any other suitable technique known to those of ordinary skill in the art.

Suitable host cells may include bacterial cells (e.g., E. coli, BL21(DE3)), but most suitably host cells are eukaryotic cells, for example insect cells (e.g. Drosophila such as Drosophila melanogaster), yeast cells, nematodes (e.g. C. elegans), mice (e.g. Mus musculus), or mammalian cells (such as Chinese hamster ovary cells (CHO) or COS cells, human 293T cells, HeLa cells, NIH 3T3 cells, and mouse erythroleukemia (MEL) cells) or human cells or other eukaryotic cells. Other suitable host cells are known to those skilled in the art. Suitably, the host cell is a mammalian cell—such as a human cell or an insect cell. In some embodiments, the suitable host cells comprise E. coli.

Other suitable host cells which may be used generally in the embodiments of the invention are those mentioned in the examples section. Vector DNA can be introduced into host cells via conventional transformation or transfection techniques. As used herein, the terms “transformation” and “transfection” are intended to refer to a variety of well-recognized techniques for introducing a foreign nucleic acid molecule (e.g., DNA) into a host cell, including calcium phosphate or calcium chloride co-precipitation, DEAE-dextran-mediated transfection, lipofection, or electroporation. Suitable methods for transforming or transfecting host cells are well known in the art.

When creating cell lines, it is generally preferred that stable cell lines are prepared. For stable transfection of mammalian cells for example, it is known that, depending upon the expression vector and transfection technique used, only a small fraction of cells may integrate the foreign DNA into their genome. In order to identify and select these integrants, a gene that encodes a selectable marker (for example, for resistance to antibiotics) is generally introduced into the host cells along with the gene of interest. Preferred selectable markers include those that confer resistance to drugs, such as G418, hygromycin, or methotrexate. Nucleic acid molecules encoding a selectable marker can be introduced into a host cell on the same vector or can be introduced on a separate vector. Cells stably transfected with the introduced nucleic acid molecule can be identified by drug selection (for example, cells that have incorporated the selectable marker gene will survive, while the other cells die).

In one embodiment, the constructs described herein are integrated into the genome of the host cell. An advantage of stable integration is that the uniformity between individual cells or clones is achieved. Another advantage is that selection of the best producers may be carried out. Accordingly, it is desirable to create stable cell lines. In another embodiment, the constructs described herein are transfected into a host cell. An advantage of transfecting the constructs into the host cell is that protein yields may be maximized. In one aspect, there is described a cell comprising the nucleic acid construct or the vector described herein.

### PD-1 Antibodies and Antigen Binding Fragments Useful in the Invention

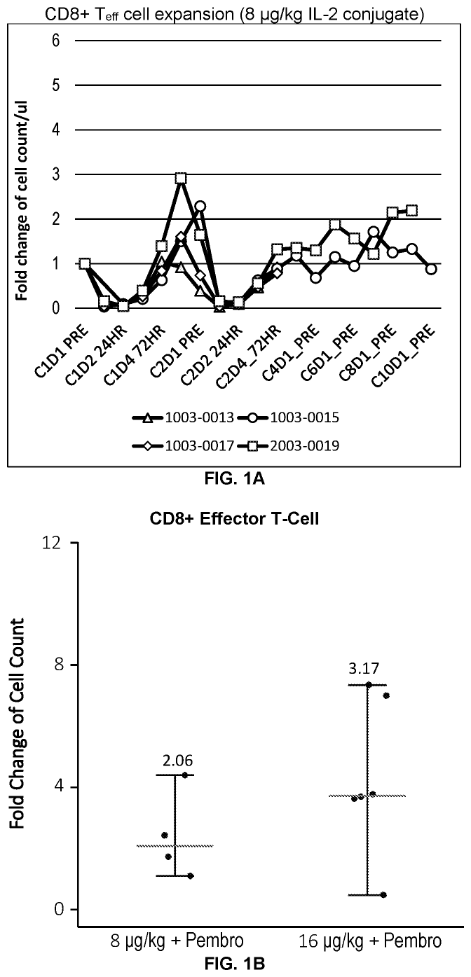

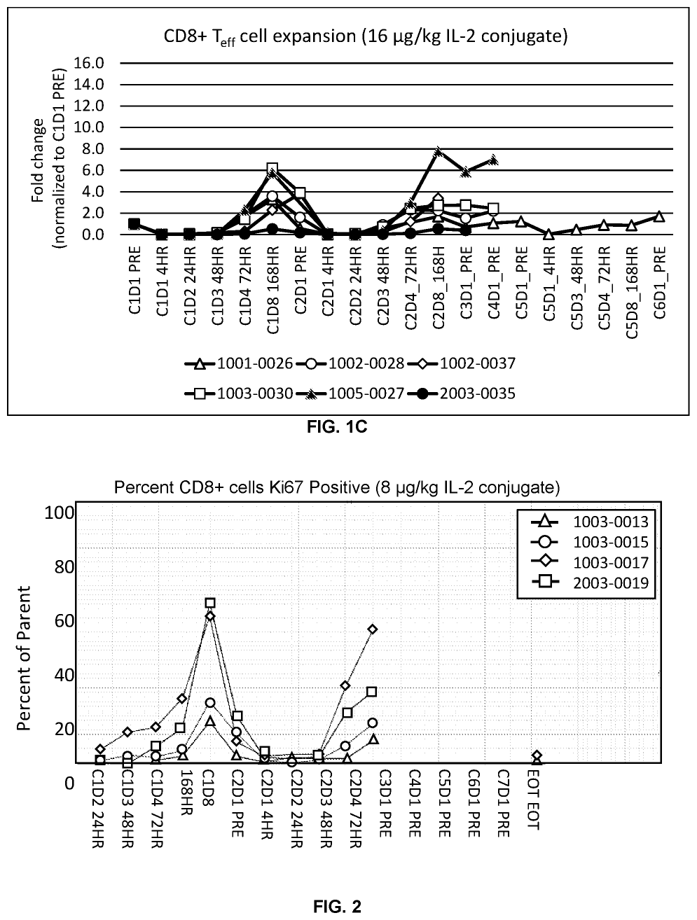

Examples of mAbs that bind to human PD-1, useful in the treatment methods and uses of the invention, are described in U.S. Pat. Nos. 7,521,051, 8,008,449, and 8,354,509. Specific anti-human PD-1 mAbs useful as a PD-1 antagonist in the treatment methods, compositions, and uses of the present invention include: pembrolizumab (formerly known as MK-3475, SCH 900475 and lambrolizumab), a humanized IgG4 mAb with the structure described in WHO Drug Information, Vol. 27, No. 2, pages 161-162 (2013) and which comprises the heavy and light chain amino acid sequences shown in FIG. 1, and the humanized antibodies h409A11, h409A16 and h409A17, which are described in WO 2008/156712 and in Table 2.

In some embodiments of the treatment methods, compositions, kits and uses of the present invention, the anti-PD-1 antibody, or antigen binding fragment thereof, comprises: (a) light chain CDRs comprising a sequence of amino acids as set forth in SEQ ID NOs: 11, 12 and 13 and heavy chain CDRs comprising a sequence of amino acids as set forth in SEQ ID NOs: 16, 17 and 18.

In some embodiments, the anti-PD-1 antibody or antigen binding fragment thereof is a human antibody. In other embodiments, the anti-PD-1 antibody or antigen binding fragment thereof is a humanized antibody. In other embodiments, the anti-PD-1 antibody or antigen binding fragment thereof is a chimeric antibody. In specific embodiments, the anti-PD-1 antibody or antigen binding fragment thereof is a monoclonal antibody.

In other embodiments of the treatment methods, compositions, kits and uses of the present invention, the anti-PD-1 antibody, or antigen binding fragment thereof, specifically binds to human PD-1 and comprises (a) a heavy chain variable region comprising an amino acid sequence as set forth in SEQ ID NO:19, or a variant thereof, and (b) a light chain variable region comprising an amino acid sequence selected from the group consisting of SEQ ID NO:14.

A variant of a heavy chain variable region sequence or full-length heavy chain sequence is identical to the reference sequence except having up to 17 conservative amino acid substitutions in the framework region (i.e., outside of the CDRs), and preferably has less than ten, nine, eight, seven, six or five conservative amino acid substitutions in the framework region. A variant of a light chain variable region sequence or full-length light chain sequence is identical to the reference sequence except having up to five conservative amino acid substitutions in the framework region (i.e., outside of the CDRs), and preferably has less than four, three or two conservative amino acid substitution in the framework region.

In another embodiment of the treatment methods, compositions, kits and uses of the present invention, the anti-PD-1 antibody or antigen-binding fragment thereof is a monoclonal antibody which specifically binds to human PD-1 and comprises (a) a heavy chain comprising or consisting of a sequence of amino acids as set forth in SEQ ID NO:20, or a variant thereof; and (b) a light chain comprising or consisting of a sequence of amino acids as set forth in SEQ ID NO:15, or a variant thereof.

In yet another embodiment of the treatment methods, compositions and uses of the invention, the anti-PD-1 antibody or antigen-binding fragment thereof is a monoclonal antibody which specifically binds to human PD-1 and comprises (a) a heavy chain comprising or consisting of a sequence of amino acids as set forth in SEQ ID NO:20 and (b) a light chain comprising or consisting of a sequence of amino acids as set forth in SEQ ID NO:15.

Table 2 below provides a list of the amino acid sequences of exemplary anti-PD-1 mAbs for use in the treatment methods, compositions, kits and uses of the present invention.

TABLE 2
Exemplary anti-human PD-1 antibodies
A. Comprises light and heavy chain CDRs of hPD-1.09A in WO2008/156712
(light and heavy chain CDRs of pembrolizumab)

| | |
|---|---|
| CDRL1 | RASKGVSTSGYSYLH |
| | SEQ ID NO: 11 |
| CDRL2 | LASYLES |
| | SEQ ID NO: 12 |
| CDRL3 | QHSRDLPLT |
| | SEQ ID NO: 13 |
| CDRH1 | NYYMY |
| | SEQ ID NO: 16 |
| CDRH2 | GINPSNGGTNFNEKFKN |
| | SEQ ID NO: 17 |
| CDRH3 | RDYRFDMGFDY |
| | SEQ ID NO: 18 |

C. Comprises the mature h109A heavy chain variable (VH) region and one
of the mature K09A light chain variable (VL) regions in WO 2008/156712

| | |
|---|---|
| Heavy chain VH | QVQLVQSGVEVKKPGASVKVSCKASGYTFTNYYMYWVRQA |
| | PGQGLEWMGGINPSNGGTNFNEKFKNRVTLTTDSSTTTAYME |
| | LKSLQFDDTAVYYCARRDYRFDMGFDYWGQGTTVTVSS |
| | SEQ ID NO: 19 (VH of pembrolizumab) |
| Light chain VL | EIVLTQSPATLSLSPGERATLSCRASKGVSTSGYSYLHWYQQK |
| | PGQAPRLLIYLASYLESGVPARFSGSGSGTDFTLTISSLEPEDFA |
| | VYYCQHSRDLPLTFGGGTKVEIK |
| | SEQ ID NO: 14 (VL of pembrolizumab) or |
| Heavy chain | QVQLVQSGVEVKKPGASVKVSCKASGYTFTNYYMYWVRQA |
| | PGQGLEWMGGINPSNGGTNFNEKFKNRVTLTTDSSTTTAYME |
| | LKSLQFDDTAVYYCARRDYRFDMGFDYWGQGTTVTVSSAST |
| | KGPSVFPLAPCSRSTSESTAALGCLVKDYFPEPVTVSWNSGAL |
| | TSGVHTFPAVLQSSGLYSLSSVVTVPSSSLGTKTYTCNVDHKP |
| | SNTKVDKRVESKYGPPCPPCPAPEFLGGPSVFLFPPKPKDTLMI |
| | SRTPEVTCVVVDVSQEDPEVQFNWYVDGVEVHNAKTKPREE |
| | QFNSTYRVVSVLTVLHQDWLNGKEYKCKVSNKGLPSSIEKTIS |
| | KAKGQPREPQVYTLPPSQEEMTKNQVSLTCLVKGFYPSDIAVE |
| | WESNGQPENNYKTTPPVLDSDGSFFLYSRLTVDKSRWQEGNV |
| | FSCSVMHEALHNHYTQKSLSLSLGK |
| | SEQ ID NO: 20 (heavy chain of pembrolizumab) |
| Light chain | EIVLTQSPATLSLSPGERATLSCRASKGVSTSGYSYLHWYQQK |
| | PGQAPRLLIYLASYLESGVPARFSGSGSGTDFTLTISSLEPEDFA |
| | VYYCQHSRDLPLTFGGGTKVEIKRTVAAPSVFIFPPSDEQLKSG |
| | TASVVCLLNNFYPREAKVQWKVDNALQSGNSQESVTEQDSK |
| | DSTYSLSSTLTLSKADYEKHKVYACEVTHQGLSSPVTKSFNRG |
| | EC |
| | SEQ ID NO: 15 (light chain of pembrolizumab) or |

In some embodiments of any of the methods and uses described herein, the method comprises administering (i) about 200 mg of an anti-PD-1 antibody or antigen binding fragment thereof to the patient every approximately three weeks or (ii) about 400 mg of an anti-PD-1 antibody, or antigen binding fragment thereof, to the patient every approximately six weeks.

In some embodiments of any of the methods, compositions, kits and uses described herein, the anti-PD-1 antibody or antigen-binding fragment thereof comprises a heavy chain variable region comprising a sequence of amino acids as set forth in SEQ ID NO:19 and a light chain variable region comprising a sequence of amino acids as set forth in SEQ ID NO:14.

In some embodiments of any of the methods, compositions, kits and uses described herein, the anti-PD-1 antibody or antigen-binding fragment thereof is a monoclonal antibody comprising (a) a heavy chain comprising a sequence of amino acids as set forth in SEQ ID NO:20, or a variant of SEQ ID NO:20, and (b) a light chain comprising a sequence of amino acids as set forth in SEQ ID NO:15, or a variant of SEQ ID NO:15.

### Methods of Treatment

In one aspect, provided herein is a method of treating a lung cancer in a subject in need thereof, comprising administering to the subject (a) an IL-2 conjugate as described herein, and (b) an anti-PD-1 antibody or antigen-binding fragment thereof, wherein the anti-PD-1 antibody or antigen-binding fragment thereof comprises light chain complementarity determining regions (CDRs) comprising a sequence of amino acids as set forth in SEQ ID NOs: 11, 12 and 13 and heavy chain CDRs comprising a sequence of amino acids as set forth in SEQ ID NOs: 16, 17 and 18 (e.g., pembrolizumab). In some embodiments, the method of treating a cancer in a subject in need thereof comprises administering to the subject (a) about 8 μg/kg of an IL-2 conjugate as described herein, and (b) an anti-PD-1 antibody or antigen-binding fragment thereof, wherein the anti-PD-1 antibody or antigen-binding fragment thereof comprises light chain complementarity determining regions (CDRs) comprising a sequence of amino acids as set forth in SEQ ID NOs: 11, 12 and 13 and heavy chain CDRs comprising a sequence of amino acids as set forth in SEQ ID NOs: 16, 17 and 18 (e.g., pembrolizumab). In some embodiments, the method of treating a cancer in a subject in need thereof comprises administering to the subject (a) about 16 μg/kg of an IL-2 conjugate as described herein, and (b) an anti-PD-1 antibody or antigen-binding fragment thereof, wherein the anti-PD-1 antibody or antigen-binding fragment thereof comprises light chain complementarity determining regions (CDRs) comprising a sequence of amino acids as set forth in SEQ ID NOs: 11, 12 and 13 and heavy chain CDRs comprising a sequence of amino acids as set forth in SEQ ID NOs: 16, 17 and 18 (e.g., pembrolizumab). In some embodiments, the method of treating a cancer in a subject in need thereof comprises administering to the subject (a) about 24 μg/kg of an IL-2 conjugate as described herein, and (b) an anti-PD-1 antibody or antigen-binding fragment thereof, wherein the anti-PD-1 antibody or antigen-binding fragment thereof comprises light chain complementarity determining regions (CDRs) comprising a sequence of amino acids as set forth in SEQ ID NOs: 11, 12 and 13 and heavy chain CDRs comprising a sequence of amino acids as set forth in SEQ ID NOs: 16, 17 and 18 (e.g., pembrolizumab). In some embodiments, the method of treating a cancer in a subject in need thereof comprises administering to the subject (a) about 32 μg/kg of an IL-2 conjugate as described herein, and (b) an anti-PD-1 antibody or antigen-binding fragment thereof, wherein the anti-PD-1 antibody or antigen-binding fragment thereof comprises light chain complementarity determining regions (CDRs) comprising a sequence of amino acids as set forth in SEQ ID NOs: 11, 12 and 13 and heavy chain CDRs comprising a sequence of amino acids as set forth in SEQ ID NOs: 16, 17 and 18 (e.g., pembrolizumab).

In another aspect, provided herein is an IL-2 conjugate for use in a method of treating a lung cancer in a subject in need thereof, the method comprising administering to the subject (a) an IL-2 conjugate as described herein, and (b) an anti-PD-1 antibody or antigen-binding fragment thereof, wherein the anti-PD-1 antibody or antigen-binding fragment thereof comprises light chain complementarity determining regions (CDRs) comprising a sequence of amino acids as set forth in SEQ ID NOs: 11, 12 and 13 and heavy chain CDRs comprising a sequence of amino acids as set forth in SEQ ID NOs: 16, 17 and 18 (e.g., pembrolizumab), wherein: the lung cancer is non-squamous non-small cell lung cancer (NSCLC), pleural mesothelioma, unresectable lung cancer, stage IV lung cancer, NSCLC having a PD-L1 tumor proportion score greater than or equal to 50%, or NSCLC having a PD-L1 tumor progession score of less than 50% or of 1-49%.

In a further aspect, provided herein is a method of treating lung cancer in a subject in need thereof, comprising: selecting a subject having lung cancer, wherein the subject is selected on the basis of one or more attributes comprising (i) the lung cancer being non-squamous non-small cell lung cancer (NSCLC); (ii) the lung cancer being pleural mesothelioma; (iii) the lung cancer being unresectable lung cancer; (iv) the lung cancer being stage IV lung cancer; (v) the lung cancer being NSCLC having a PD-L1 tumor proportion score greater than or equal to 50%; (vi) the lung cancer being NSCLC having a PD-L1 tumor progession score of less than 50% or of 1-49%; and administering to the subject (a) an IL-2 conjugate as described herein, and (b) an anti-PD-1 antibody or antigen-binding fragment thereof, wherein the anti-PD-1 antibody or antigen-binding fragment thereof comprises light chain complementarity determining regions (CDRs) comprising a sequence of amino acids as set forth in SEQ ID NOs: 11, 12 and 13 and heavy chain CDRs comprising a sequence of amino acids as set forth in SEQ ID NOs: 16, 17 and 18 (e.g., pembrolizumab).

In a further aspect, provided herein is a method of treating lung cancer in a subject in need thereof, comprising administering to the subject (a) an IL-2 conjugate described herein, (b) an anti-PD-1 antibody or antigen-binding fragment thereof, wherein the anti-PD-1 antibody or antigen-binding fragment thereof comprises light chain complementarity determining regions (CDRs) comprising a sequence of amino acids as set forth in SEQ ID NOs: 11, 12 and 13 and heavy chain CDRs comprising a sequence of amino acids as set forth in SEQ ID NOs: 16, 17 and 18 (e.g., pembrolizumab), and (c) cisplatin.

In a further aspect, provided herein is use of an IL-2 conjugate for the manufacture of a medicament for a method disclosed herein of treating a cancer in a subject in need thereof.

The following embodiments apply to any of the foregoing aspects. In some embodiments, the method comprises administering to the subject (a) about 8 μg/kg of an IL-2 conjugate as described herein, and (b) an anti-PD-1 antibody or antigen-binding fragment thereof, wherein the anti-PD-1 antibody or antigen-binding fragment thereof comprises light chain complementarity determining regions (CDRs) comprising a sequence of amino acids as set forth in SEQ ID NOs: 11, 12 and 13 and heavy chain CDRs comprising a sequence of amino acids as set forth in SEQ ID NOs: 16, 17 and 18 (e.g., pembrolizumab). In some embodiments, the method comprises administering to the subject (a) about 16 μg/kg of an IL-2 conjugate as described herein, and (b) an anti-PD-1 antibody or antigen-binding fragment thereof, wherein the anti-PD-1 antibody or antigen-binding fragment thereof comprises light chain complementarity determining regions (CDRs) comprising a sequence of amino acids as set forth in SEQ ID NOs: 11, 12 and 13 and heavy chain CDRs comprising a sequence of amino acids as set forth in SEQ ID NOs: 16, 17 and 18 (e.g., pembrolizumab). In some embodiments, the method comprises administering to the subject (a) about 24 μg/kg of an IL-2 conjugate as described herein, and (b) an anti-PD-1 antibody or antigen-binding fragment thereof (e.g., pembrolizumab). In some embodiments, the method comprises administering to the subject (a) about 32 μg/kg of an IL-2 conjugate as described herein, and (b) an anti-PD-1 antibody or antigen-binding fragment thereof, wherein the anti-PD-1 antibody or antigen-binding fragment thereof comprises light chain complementarity determining regions (CDRs) comprising a sequence of amino acids as set forth in SEQ ID NOs: 11, 12 and 13 and heavy chain CDRs comprising a sequence of amino acids as set forth in SEQ ID NOs: 16, 17 and 18 (e.g., pembrolizumab).

The embodiments described in the following sections apply to any of the foregoing aspects.

### Cancer Types

In some embodiments, the lung cancer is non-small cell lung cancer (NSCLC). In some embodiments, the lung cancer is unresectable. In some embodiments, the lung cancer is stage IV, such as stage IV NSCLC. In some embodiments, the lung cancer is non-squamous NSCLC, such as stage IV non-squamous NSCLC. In some embodiments, the lung cancer is pleural mesothelioma, such as unresectable pleural mesothelioma. In some embodiments, the lung cancer is NSCLC adenocarcinoma.

In some embodiments, the lung cancer (e.g., NSCLC) has a PD-L1 tumor proportion score greater than or equal to 50%. In some embodiments, the lung cancer (e.g., NSCLC) has a PD-L1 tumor proportion score of less than 50%. In some embodiments, the lung cancer (e.g., NSCLC) has a PD-L1 tumor proportion score of 1-49%. PD-L1 tumor proportion score is the percentage of viable tumor cells showing membrane PD-L1 staining relative to all viable tumor cells. See, e.g., Sim et al., Korean J Intern Med. 2018 July; 33(4): 737-744.

### Administration

In some embodiments, the IL-2 conjugate is administered to the subject by intravenous, subcutaneous, intramuscular, intracerebral, intranasal, intra-arterial, intra-articular, intradermal, intravitreal, intraosseous infusion, intraperitoneal, or intrathecal administration. In some embodiments, the IL-2 conjugate is administered to the subject by intravenous, subcutaneous, or intramuscular administration. In some embodiments, the IL-2 conjugate is administered to the subject by intravenous administration. In some embodiments, the IL-2 conjugate is administered to the subject by subcutaneous administration. In some embodiments, the IL-2 conjugate is administered to the subject by intramuscular administration. In some embodiments, the IL-2 conjugate and the anti-PD-1 antibody or antigen-binding fragment thereof are administered to the subject by intravenous administration. In some embodiments, the IL-2 conjugate and the anti-PD-1 antibody or antigen-binding fragment thereof are administered to the subject by subcutaneous administration.

The IL-2 conjugate may be administered more than once, e.g., twice, three times, four times, five times, or more. In some embodiments, the duration of the treatment is up to 24 months, such as 1 month, 2 months, 3 months, 6 months, 9 months, 12 months, 15 months, 18 months, 21 months or 24 months. In some embodiments, the duration of treatment is further extended by up to another 24 months. In some embodiments, the duration of treatment is up to 35 cycles. In some embodiments, the duration of treatment is until progressive disease.

In some embodiments, the IL-2 conjugate is administered to the subject separately from the administration of the anti-PD-1 antibody or antigen-binding fragment thereof. In some embodiments, the IL-2 conjugate and the anti-PD-1 antibody or antigen-binding fragment thereof are administered to the subject sequentially. In some embodiments, the IL-2 conjugate is administered to the subject prior to the administration to the subject of the anti-PD-1 antibody or antigen-binding fragment thereof. In some embodiments, the IL-2 conjugate is administered to the subject after the administration to the subject of the anti-PD-1 antibody or antigen-binding fragment thereof. In some embodiments, the IL-2 conjugate and the anti-PD-1 antibody or antigen-binding fragment thereof are administered to the subject simultaneously.

In some embodiments, the IL-2 conjugate is administered to a subject in need thereof about once every two weeks, about once every three weeks, or about once every 4 weeks. In some embodiments, the IL-2 conjugate is administered to a subject in need thereof once every two weeks. In some embodiments, the IL-2 conjugate is administered to a subject in need thereof once every three weeks. In some embodiments, the IL-2 conjugate is administered to a subject in need thereof once every 4 weeks. In some embodiments, the IL-2 conjugate is administered about once every 14, 15, 16, 17, 18, 19, 20, or 21 days.

In some embodiments, the anti-PD-1 antibody or antigen-binding fragment thereof is administered to a subject in need thereof about once every two weeks, about once every three weeks, or about once every 4 weeks. In some embodiments, the anti-PD-1 antibody or antigen-binding fragment thereof is administered to a subject in need thereof once every two weeks. In some embodiments, the anti-PD-1 antibody or antigen-binding fragment thereof is administered to a subject in need thereof once every three weeks. In some embodiments, the anti-PD-1 antibody or antigen-binding fragment thereof is administered to a subject in need thereof once every 4 weeks. In some embodiments, the anti-PD-1 antibody or antigen-binding fragment thereof is administered about once every 14, 15, 16, 17, 18, 19, 20, or 21 days.

In some embodiments, the IL-2 conjugate and the anti-PD-1 antibody or antigen-binding fragment thereof are administered to a subject in need thereof about once every two weeks, about once every three weeks, or about once every 4 weeks. In some embodiments, the IL-2 conjugate and the anti-PD-1 antibody or antigen-binding fragment thereof are administered to a subject in need thereof once every two weeks. In some embodiments, the IL-2 conjugate and the anti-PD-1 antibody or antigen-binding fragment thereof are administered to a subject in need thereof once every three weeks. In some embodiments, the IL-2 conjugate and the anti-PD-1 antibody or antigen-binding fragment thereof are administered to a subject in need thereof once every 4 weeks. In some embodiments, the IL-2 conjugate and the anti-PD-1 antibody or antigen-binding fragment thereof are administered about once every 14, 15, 16, 17, 18, 19, 20, or 21 days.

In some instances, the desired doses are conveniently presented in a single dose or as divided doses administered simultaneously (or over a short period of time) or at appropriate intervals, for example as two, three, four or more sub-doses per day.

In some embodiments, the anti-PD-1 antibody or antigen-binding fragment thereof is administered at a dose of about 200 mg every 3 weeks.

In some embodiments, a method described herein further comprises administering one or more additional therapeutic agents. In some embodiments, the additional therapeutic agent comprises a chemotherapeutic agent. In some embodiments, the chemotherapeutic agent comprises pemetrexed. In some embodiments, the chemotherapeutic agent comprises a platinum agent, such as carboplatin. In some embodiments, the chemotherapeutic agent comprises cisplatin. In some embodiments, the chemotherapeutic agent comprises nab-paclitaxel. In some embodiments, the chemotherapeutic agent comprises pemetrexed and a platinum agent, such as carboplatin. In some embodiments, the chemotherapeutic agent comprises pemetrexed and cisplatin.

In some embodiments, the additional therapeutic agent comprises an antihistamine, such as diphenhydramine. In some embodiments, the additional therapeutic agent comprises a chemotherapeutic agent and an antihistamine, such as diphenhydramine. In some embodiments, the additional therapeutic agent comprises any one of the foregoing chemotherapeutic agents and an antihistamine, such as diphenhydramine.

In some embodiments, the additional therapeutic agent comprises an analgesic, such as acetaminophen. In some embodiments, the additional therapeutic agent comprises a chemotherapeutic agent and an analgesic, such as acetaminophen. In some embodiments, the additional therapeutic agent comprises any one of the foregoing chemotherapeutic agents and an analgesic, such as acetaminophen.

In some embodiments, the additional therapeutic agent comprises one or more vitamins, such as folic acid and/or vitamin B12. In some embodiments, the additional therapeutic agent comprises a chemotherapeutic agent and one or more vitamins, such as folic acid and/or vitamin B12. In some embodiments, the additional therapeutic agent comprises any one of the foregoing chemotherapeutic agents and one or more vitamins, such as folic acid and/or vitamin B12.

In some embodiments, the additional therapeutic agent comprises an antihistamine and an analgesic, such as diphenhydramine and acetaminophen. In some embodiments, the additional therapeutic agent comprises an antihistamine and one or more vitamins, such as diphenhydramine and one or both of folic acid and vitamin B12. In some embodiments, the additional therapeutic agent comprises an analgesic and one or more vitamins, such as acetaminophen and one or both of folic acid and vitamin B12. In some embodiments, the additional therapeutic agent comprises an antihistamine, an analgesic, and one or more vitamins, such as diphenhydramine, acetaminophen, and one or both of folic acid and vitamin B12. In any of the foregoing embodiments, the additional therapeutic agent can further comprise a chemotherapeutic agent, such as any one of the foregoing chemotherapeutic agents.

### Subject

In some embodiments, administration of the IL-2 conjugate and the anti-PD-1 antibody or antigen-binding fragment thereof is to an adult. In some embodiments, the adult is a male. In other embodiments, the adult is a female. In some embodiments, the subject is 18 years of age or older. In some embodiments, the adult is at least age 18, 20, 25, 30, 35, 40, 45, 50, 55, 60, 65, 70, 75, 80, 85, 90, or 95 years of age.

In some embodiments, the subject has NSCLC having a PD-L1 tumor proportion score (TPS) greater than or equal to 50% and has not received prior treatment. That is, the subject will receive the IL-2 conjugate treatment as 1L or first-line therapy. In some embodiments, the subject is a 1L NSCLC subject. In some embodiments, the subject is a treatment-naïve NSCLC subject. In some embodiments, the subject is a 1L NSCLC subject having a TPS greater than or equal to 50%. In some embodiments, the subject is a treatment-naïve NSCLC subject having a TPS greater than or equal to 50%. In any of the foregoing embodiments, the NSCLC may be stage IV.

In some embodiments, the subject has NSCLC having a PD-L1 tumor proportion score (TPS) of 1-49% and has not received prior treatment. That is, the subject will receive the IL-2 conjugate treatment as 1L or first-line therapy. In some embodiments, the subject is a 1L NSCLC subject. In some embodiments, the subject is a treatment-naïve NSCLC subject. In some embodiments, the subject is a 1L NSCLC subject having a TPS of 1-49%. In some embodiments, the subject is a treatment-naïve NSCLC subject having a TPS of 1-49%. In any of the foregoing embodiments, the NSCLC may be stage IV.

In some embodiments, the subject has non-squamous NSCLC and has not received prior treatment, and treatment includes administration of (1) pemetrexed and (2) carboplatin and/or cisplatin. That is, the subject will receive the IL-2 conjugate treatment as 1L or first-line therapy. In some embodiments, the subject is a 1L non-squamous NSCLC subject. In some embodiments, the subject is a treatment-naïve non-squamous NSCLC subject. In some embodiments, the subject is a 1L NSCLC subject. In some embodiments, the subject is a treatment-naïve NSCLC subject. In any of the foregoing embodiments, the NSCLC may be stage IV.

In some embodiments, the subject has NSCLC, has received 1 or 2 prior lines of therapy, and has progressed on a checkpoint inhibitor (CPI)-based therapy, such as PD-1/PD-L1. That is, the subject will receive the IL-2 conjugate treatment as 2/3L or second- or third-line therapy. In some embodiments, the subject is a 2L or 3L NSCLC subject. In some embodiments, the subject is a 2L NSCLC subject. In some embodiments, the subject is a 3L NSCLC subject. In any of the foregoing embodiments, the NSCLC may be stage IV. In some embodiments, the subject received one anti-PD-1/PD-L1 containing regimen which included chemotherapy agents as part of the regimen to treat stage IV NSCLC which progressed, after documented benefit, on an anti-PD-1/PD-L1 containing regimen per RECIST 1.1. In some embodiments, the documentation of benefit from an anti-PD-1/PD-L1 containing regimen is defined as SD at ≥1 radiographic imaging scan, CR, or partial response (PR). In some embodiments, the anti-PD-1/PD-L1 containing regimen is an anti-PD-1/PD-L1 monotherapy. In some embodiments, the anti-PD-1/PD-L1 containing regimen is an anti-PD-1/PD-L1 agent administered in the same cycle as another systemic anticancer therapy. In some embodiments, the PD-1/PD-L1 treatment was used beyond initial radiological progression while continuing treatment with the same PD-1/PD-L1 agent used before PD. In some embodiments, the subject received the platinum-based chemotherapy as part of the anti-PD-1/PD-L1 containing regimen. In some embodiments, the subject received the platinum-based chemotherapy as a separate regimen. In some embodiments, the subject received no more than one previous chemotherapy regimen. In some embodiments, the subject received one or two previous chemotherapy regiments and the prior anti-PD-1/PD-L1 containing regimen did not include platinum-based chemotherapy. In some embodiments, the subject received no more than 2 prior chemotherapy treatments. In some embodiments, the subject declined platinum-based chemotherapy. In some embodiments, the subject cannot tolerate platinum-based chemotherapy.

In some embodiments, the subject has NSCLC, has received 1 or 2 prior lines of therapy, and has progressed on a checkpoint inhibitor (CPI)-based therapy, such as PD-1/PD-L1, and treatment includes administration of nab-paclitaxel. That is, the subject will receive the IL-2 conjugate treatment as 2/3L or second- or third-line therapy. In some embodiments, the subject is a 2L or 3L NSCLC subject. In some embodiments, the subject is a 2L NSCLC subject. In some embodiments, the subject is a 3L NSCLC subject. In some embodiments, the subject is a 2L or 3L non-squamous NSCLC subject. In any of the foregoing embodiments, the NSCLC may be stage IV. In some embodiments, the subject received one anti-PD-1/PD-L1 containing regimen which included chemotherapy agents as part of the regimen to treat stage IV NSCLC which progressed, after documented benefit, on an anti-PD-1/PD-L1 containing regimen per RECIST 1.1. In some embodiments, the documentation of benefit from an anti-PD-1/PD-L1 containing regimen is defined as SD at ≥1 radiographic imaging scan, CR, or partial response (PR). In some embodiments, the anti-PD-1/PD-L1 containing regimen is an anti-PD-1/PD-L1 monotherapy. In some embodiments, the anti-PD-1/PD-L1 containing regimen is an anti-PD-1/PD-L1 agent administered in the same cycle as another systemic anticancer therapy. In some embodiments, the PD-1/PD-L1 treatment was used beyond initial radiological progression while continuing treatment with the same PD-1/PD-L1 agent used before PD. In some embodiments, the subject received the platinum-based chemotherapy as part of the anti-PD-1/PD-L1 containing regimen. In some embodiments, the subject received the platinum-based chemotherapy as a separate regimen. In some embodiments, the subject received no more than one previous chemotherapy regimen. In some embodiments, the subject received one or two previous chemotherapy regiments and the prior anti-PD-1/PD-L1 containing regimen did not include platinum-based chemotherapy. In some embodiments, the subject received no more than 2 prior chemotherapy treatments. In some embodiments, the subject declines platinum-based chemotherapy. In some embodiments, the subject cannot tolerate platinum-based chemotherapy.

In some embodiments, the subject has unresectable malignant pleural mesothelioma, has received 1 or 2 prior lines of therapy, and is checkpoint inhibitor (CPI) naïve. That is, the subject will receive the IL-2 conjugate treatment as 2/3L or second- or third-line therapy. In some embodiments, the subject is a 2L or 3L unresectable malignant pleural mesothelioma subject. In some embodiments, the subject is a 2L unresectable malignant pleural mesothelioma subject. In some embodiments, the subject is a 3L unresectable malignant pleural mesothelioma subject. In some embodiments, the subject is a 2L or 3L mesothelioma subject.

In some embodiments, the subject has measurable disease (i.e., cancer). Measureable disease may be determined by RECIST v1.1. In some embodiments, the subject has been determined to have Eastern Cooperative Oncology Group (ECOG) performance status of <2, e.g., 0 or 1. In some embodiments, the subject has adequate cardiovascular, hematological, liver, renal function, and laboratory parameters, as determined by a physician. In some embodiments, the subject has been determined (e.g., by a physician) to have a life expectancy greater than or equal to 12 weeks. In some embodiments, the subject has had prior anti-cancer therapy before administration of the first treatment dose.

In some embodiments, the subject does not have a history of allogenic tissue/solid organ transplant. In some embodiments, the subject does not have immune-mediated/related toxicity from prior immune-oncology therapy of Grade 4 or leading to discontinuation. In some embodiments, the subject does not have ongoing AEs caused by any prior anti-cancer therapy greater than or equal to 2. In some embodiments, the subject does not have baseline oxygen saturation (SpO2) less than or equal to 92% (without oxygen therapy). In some embodiments, the subject has not received prior IL-2-based anti-cancer treatment. In some embodiments, the subject can temporarily (for at least 36 hours) withhold antihypertensive medications prior to each IL-2 conjugate dosing. In some embodiments, the subject does not have any medical or clinical condition, laboratory abnormality, or any specific situation that would preclude treatment according to the methods disclosed herein, as determined by the supervising physician. In some embodiments, the subject does not have a comorbidity requiring corticosteroid therapy. In some embodiments, the subject does not have active brain metastases or leptomeningeal disease. In some embodiments, the subject did not receive major surgery or local intervention within 28 days of IL-2 conjugate treatment. In some embodiments, the subject did not receive a last administration of a prior antitumor therapy or investigation treatment within 28 days or within 5 times the half-life, whichever is shorter, of IL-2 conjugate treatment. In some embodiments, the subject did not receive antibiotics (excluding topical antibiotics) within 14 days of the first administration of the IL-2 conjugate. In some embodiments, the subject does not have a severe or unstable cardiac condition within 6 months starting treatment with the IL-2 conjugate. In some embodiments, the subject does not have active, known, or suspected autoimmune disease that has required systemic treatment within 2 years of starting treatment with the IL-2 conjugate. In some embodiments, the subject does not have a known second malignancy either progressing or requiring active treatment within 3 years of starting treatment with the IL-2 conjugate. In some embodiments, the subject has not received a live-virus or live-attenuated vaccination within 28 days of starting treatment with the IL-2 conjugate.

In some embodiments, wherein the subject has NSCLC having a PD-L1 tumor proportion score (TPS) greater than or equal to 50% and has not received prior treatment, the subject has at least one measurable lesion per RECIST v1.1. In some embodiments, wherein the subject has NSCLC having a PD-L1 tumor proportion score (TPS) greater than or equal to 50% and has not received prior treatment, the subject has histologically or cytologically confirmed diagnosis of Stage IV NSCLC. In some embodiments, wherein the subject has NSCLC having a PD-L1 tumor proportion score (TPS) greater than or equal to 50% and has not received prior treatment, the subject does not have known driver alterations, such as epidermal growth factor receptor (EGFR), anaplastic lymphoma kinase (ALK), proto-oncogene tyrosine-protein kinase (ROS)1, or BRAF mutation. In any of the foregoing embodiments, the NSCLC may be stage IV.

In some embodiments, wherein the subject has NSCLC having a PD-L1 tumor proportion score (TPS) of 1-49% and has not received prior treatment, the subject has at least one measurable lesion per RECIST v1.I. In some embodiments, wherein the subject has NSCLC having a PD-L1 tumor proportion score (TPS) of 1-49% and has not received prior treatment, the subject has histologically or cytologically confirmed diagnosis of Stage IV NSCLC. In some embodiments, wherein the subject has NSCLC having a PD-L1 tumor proportion score (TPS) of 1-49% and has not received prior treatment, the subject does not have known driver alterations, such as epidermal growth factor receptor (EGFR), anaplastic lymphoma kinase (ALK), proto-oncogene tyrosine-protein kinase (ROS)1, or BRAF mutation. In any of the foregoing embodiments, the NSCLC may be stage IV.

In some embodiments, wherein the subject has non-squamous NSCLC and has not received prior treatment, and wherein treatment includes administration of pemetrexed and carboplatin/cisplatin, the subject has at least one measurable lesion per RECIST v1.1. In some embodiments, wherein the subject has non-squamous NSCLC and has not received prior treatment, the subject has histologically or cytologically confirmed diagnosis of Stage IV non-squamous NSCLC. In some embodiments, wherein the subject has non-squamous NSCLC and has not received prior treatment, the subject does not have known driver alterations, such as epidermal growth factor receptor (EGFR), anaplastic lymphoma kinase (ALK), proto-oncogene tyrosine-protein kinase (ROS)1, or BRAF mutation. In some embodiments, wherein the subject has non-squamous NSCLC and has not received prior treatment, the subject does not have uncontrolled pleural/peritoneal effusion, pericardial effusion or ascites requiring recurrent drainage procedures; predominantly squamous cell histology NSCLC; or inability to interrupt aspirin or other nonsteroidal anti-inflammatory drugs (NSAIDs), other than an aspirin dose less than or equal to 1.3 g per day, for a 5-day period.

In some embodiments, wherein the subject has NSCLC, has received 1 or 2 prior lines of therapy, and has progressed on a checkpoint inhibitor (CPI)-based therapy, such as PD-1/PD-L1, the subject has at least one measurable lesion per RECIST v1.1. In some embodiments, wherein the subject has NSCLC, has received 1 or 2 prior lines of therapy, and has progressed on a checkpoint inhibitor (CPI)-based therapy, such as PD-1/PD-L1, the subject has histologically or cytologically confirmed diagnosis of Stage IV NSCLC. In some embodiments, wherein the subject has NSCLC, has received 1 or 2 prior lines of therapy, and has progressed on a checkpoint inhibitor (CPI)-based therapy, such as PD-1/PD-L1, the subject does not have known driver alterations, such as epidermal growth factor receptor (EGFR), anaplastic lymphoma kinase (ALK), proto-oncogene tyrosine-protein kinase (ROS)1, or BRAF mutation. In some embodiments, the subject previously received one PD-1/PD-L1 treatment regimen, which was administered concurrently or sequentially with a platinum-based chemotherapy, plus one additional chemotherapy regimen.

In some embodiments, wherein the subject has NSCLC, has received 1 or 2 prior lines of therapy, and has progressed on a checkpoint inhibitor (CPI)-based therapy, such as PD-1/PD-L1, and wherein treatment includes administration of nab-paclitaxel, the subject has at least one measurable lesion per RECIST v1.1. In some embodiments, wherein the subject has NSCLC, has received 1 or 2 prior lines of therapy, and has progressed on a checkpoint inhibitor (CPI)-based therapy, such as PD-1/PD-L1, and wherein treatment includes administration of nab-paclitaxel, the subject has histologically or cytologically confirmed diagnosis of Stage IV NSCLC. In some embodiments, wherein the subject has NSCLC, has received 1 or 2 prior lines of therapy, and has progressed on a checkpoint inhibitor (CPI)-based therapy, such as PD-1/PD-L1, and wherein treatment includes administration of nab-paclitaxel, the subject does not have known driver alterations, such as epidermal growth factor receptor (EGFR), anaplastic lymphoma kinase (ALK), proto-oncogene tyrosine-protein kinase (ROS)1, or BRAF mutation. In some embodiments, the subject previously received one PD-1/PD-L1 treatment regimen, which was administered concurrently or sequentially with a platinum-based chemotherapy, plus one additional chemotherapy regimen.

In some embodiments, wherein the subject has unresectable malignant pleural mesothelioma, has received 1 or 2 prior lines of therapy, and is checkpoint inhibitor (CPI) naïve, the subject has at least one measurable lesion per modified RECIST. In some embodiments, wherein the subject has mesothelioma, has received 1 or 2 prior lines of therapy, and is checkpoint inhibitor (CPI) naïve, the subject has histologically confirmed unresectable malignant pleural mesothelioma.

In some embodiments, the subject is a female that is not pregnant or breastfeeding. In some embodiments, the subject is a female that is not of childbearing potential (WOCBP). In some embodiments, the subject is a female that is of childbearing potential (WOCBP) and using an approved contraception method for at least 150 days after discontinuing IL-2 conjugate treatment. In some embodiments, the subject is a female that is of childbearing potential (WOCBP) and using an approved contraception method for at least 420 days after discontinuing IL-2 conjugate treatment. In some embodiments, the subject is a female that is of childbearing potential (WOCBP) and does not donate or cryopreserve eggs for at least 150 days after discontinuing IL-2 conjugate treatment. In some embodiments, the subject is a male that does not donate or crypreserve sperm. In some embodiments, the subject is a male who abstains from heterosexual intercourse at least 330 days after discontinuing IL-2 conjugate treatment. In some embodiments, the subject is a male who abstains from heterosexual intercourse at least 210 days after discontinuing IL-2 conjugate treatment. In some embodiments, the subject is a male who uses an approved contraception for at least 330 days after discontinuing IL-2 conjugate treatment. In some embodiments, the subject is a male who uses an approved contraception for at least 210 days after discontinuing IL-2 conjugate treatment.

In some embodiments, the subject has no known hypersensitivity or contraindications to any of the IL-2 conjugates disclosed herein, PEG, pegylated drugs, or the anti-PD-1 antibody or antigen-binding fragment thereof. In some embodiments, the subject has not received a previous anticancer treatment comprising IL-2.

In some embodiments, the subject is selected to receive the IL-2 conjugate and the anti-PD-1 antibody or antigen-binding fragment thereof on the basis of one or more attributes comprising (i) the lung cancer being non-squamous non-small cell lung cancer (NSCLC); (ii) the lung cancer being pleural mesothelioma; (iii) the lung cancer being unresectable lung cancer; (iv) the lung cancer being stage IV lung cancer; (v) the lung cancer being NSCLC having a PD-L1 tumor proportion score greater than or equal to 50%; or (vi) the lung cancer being NSCLC having a PD-L1 tumor progession score of less than 50% or of 1-49%.

In some embodiments, the subject is selected to receive the IL-2 conjugate and the anti-PD-1 antibody or antigen-binding fragment thereof at least in part on the basis of the lung cancer being non-squamous NSCLC (e.g., stage IV non-squamous NSCLC). In some embodiments, the subject is selected to receive the IL-2 conjugate and the anti-PD-1 antibody or antigen-binding fragment thereof at least in part on the basis of the lung cancer being stage IV NSCLC. In some embodiments, the subject is selected to receive the IL-2 conjugate and the anti-PD-1 antibody or antigen-binding fragment thereof at least in part on the basis of the lung cancer being pleural mesothelioma. In some embodiments, the subject is selected to receive the IL-2 conjugate and the anti-PD-1 antibody or antigen-binding fragment thereof at least in part on the basis of the lung cancer being unresectable (e.g., unresectable pleural mesothelioma). In some embodiments, the subject is selected to receive the IL-2 conjugate and the anti-PD-1 antibody or antigen-binding fragment thereof at least in part on the basis of the lung cancer being stage IV lung cancer. In some embodiments, the subject is selected to receive the IL-2 conjugate and the anti-PD-1 antibody or antigen-binding fragment thereof at least in part on the basis of the lung cancer having a PD-L1 tumor proportion score greater than or equal to 50%. In some embodiments, the subject is selected to receive the IL-2 conjugate and the anti-PD-1 antibody or antigen-binding fragment thereof at least in part on the basis of the lung cancer having a PD-L1 tumor proportion score less than 50%. In some embodiments, the subject is selected to receive the IL-2 conjugate and the anti-PD-1 antibody or antigen-binding fragment thereof at least in part on the basis of the lung cancer having a PD-L1 tumor proportion score from 1 to 49%.

### Effects of Administration

In some embodiments, administration of the IL-2 conjugate and the anti-PD-1 antibody or antigen-binding fragment thereof provides a complete response, a partial response or stable disease. In some embodiments, administration of the IL-2 conjugate and the anti-PD-1 antibody or antigen-binding fragment thereof provides a complete response. In some embodiments, administration of the IL-2 conjugate and the anti-PD-1 antibody or antigen-binding fragment thereof provides a partial response. In some embodiments, administration of the IL-2 conjugate and the anti-PD-1 antibody or antigen-binding fragment thereof provides stable disease.

In some embodiments, administration of the IL-2 conjugate and the anti-PD-1 antibody or antigen-binding fragment thereof provides a decrease in the size of target lesions. In some embodiments, administration of the IL-2 conjugate and the anti-PD-1 antibody or antigen-binding fragment thereof stabilizes the size of target lesions. In some variations, administration of the IL-2 conjugate and the anti-PD-1 antibody or antigen-binding fragment thereof slows down the growth rate of target lesions. In some variations, administration of the IL-2 conjugate and the anti-PD-1 antibody or antigen-binding fragment thereof stops the growth of target lesions. In some embodiments, administration of the IL-2 conjugate and the anti-PD-1 antibody or antigen-binding fragment thereof eliminates the target lesions.

In some embodiments, following administration of the IL-2 conjugate and the anti-PD-1 antibody or antigen-binding fragment thereof, the subject experiences a response as measured by the Immune-related Response Evaluation Criteria in Solid Tumors (iRECIST). In some embodiments, following administration of the IL-2 conjugate and the anti-PD-1 antibody or antigen-binding fragment thereof, the subject experiences an Objective Response Rate (ORR) according to RECIST version 1.1. In some embodiments, following administration of the IL-2 conjugate and the anti-PD-1 antibody or antigen-binding fragment thereof, the subject experiences Duration of Response (DOR) according to RECIST versions 1.1. In some embodiments, following administration of the IL-2 conjugate and the anti-PD-1 antibody or antigen-binding fragment thereof, the subject experiences Progression-Free Survival (PFS) according to RECIST version 1.1. In some embodiments, following administration of the IL-2 conjugate and the anti-PD-1 antibody or antigen-binding fragment thereof, the subject experiences Overall Survival according to RECIST version 1.1. In some embodiments, following administration of the IL-2 conjugate and the anti-PD-1 antibody or antigen-binding fragment thereof, the subject experiences Time to Response (TTR) according to RECIST version 1.1. In some embodiments, following administration of the IL-2 conjugate and the anti-PD-1 antibody or antigen-binding fragment thereof, the subject experiences Disease Control Rate (DCR) according to RECIST version 1.1. In any of these embodiments, the subject's experience is based on a physician's review of a radiographic image taken of the subject.

In some embodiments, administration of the IL-2 conjugate and the anti-PD-1 antibody or antigen-binding fragment thereof to the subject does not cause vascular leak syndrome in the subject. In some embodiments, administration of the IL-2 conjugate and the anti-PD-1 antibody or antigen-binding fragment thereof to the subject does not cause Grade 2, Grade 3, or Grade 4 vascular leak syndrome in the subject. In some embodiments, administration of the IL-2 conjugate and the anti-PD-1 antibody or antigen-binding fragment thereof to the subject does not cause Grade 2 vascular leak syndrome in the subject. In some embodiments, administration of the IL-2 conjugate and the anti-PD-1 antibody or antigen-binding fragment thereof to the subject does not cause Grade 3 vascular leak syndrome in the subject. In some embodiments, administration of the IL-2 conjugate and the anti-PD-1 antibody or antigen-binding fragment thereof to the subject does not cause Grade 4 vascular leak syndrome in the subject. In some embodiments, administration of the IL-2 conjugate and the anti-PD-1 antibody or antigen-binding fragment thereof to the subject does not cause loss of vascular tone in the subject.

In some embodiments, administration of the IL-2 conjugate and the anti-PD-1 antibody or antigen-binding fragment thereof to the subject does not cause extravasation of plasma proteins and fluid into the extravascular space in the subject.

In some embodiments, administration of the IL-2 conjugate and the anti-PD-1 antibody or antigen-binding fragment thereof to the subject does not cause hypotension and reduced organ perfusion in the subject.

In some embodiments, administration of the IL-2 conjugate and the anti-PD-1 antibody or antigen-binding fragment thereof to the subject does not cause impaired neutrophil function in the subject. In some embodiments, administration of the IL-2 conjugate and the anti-PD-1 antibody or antigen-binding fragment thereof to the subject does not cause reduced chemotaxis in the subject.

In some embodiments, administration of the IL-2 conjugate and the anti-PD-1 antibody or antigen-binding fragment thereof to the subject is not associated with an increased risk of disseminated infection in the subject. In some embodiments, the disseminated infection is sepsis or bacterial endocarditis. In some embodiments, the disseminated infection is sepsis. In some embodiments, the disseminated infection is bacterial endocarditis. In some embodiments, the subject is treated for any preexisting bacterial infections prior to administration of the IL-2 conjugate and the anti-PD-1 antibody or antigen-binding fragment thereof. In some embodiments, the subject is treated with an antibacterial agent selected from oxacillin, nafcillin, ciprofloxacin, and vancomycin prior to administration of the IL-2 conjugate and the anti-PD-1 antibody or antigen-binding fragment thereof.

In some embodiments, administration of the IL-2 conjugate and the anti-PD-1 antibody or antigen-binding fragment thereof to the subject does not exacerbate a pre-existing or initial presentation of an autoimmune disease or an inflammatory disorder in the subject. In some embodiments, the administration of the IL-2 conjugate and the anti-PD-1 antibody or antigen-binding fragment thereof to the subject does not exacerbate a pre-existing or initial presentation of an autoimmune disease in the subject. In some embodiments, the administration of the IL-2 conjugate and the anti-PD-1 antibody or antigen-binding fragment thereof to the subject does not exacerbate a pre-existing or initial presentation of an inflammatory disorder in the subject. In some embodiments, the autoimmune disease or inflammatory disorder in the subject is selected from Crohn's disease, scleroderma, thyroiditis, inflammatory arthritis, diabetes mellitus, oculo-bulbar myasthenia gravis, crescentic IgA glomerulonephritis, cholecystitis, cerebral vasculitis, Stevens-Johnson syndrome and bullous pemphigoid. In some embodiments, the autoimmune disease or inflammatory disorder in the subject is Crohn's disease. In some embodiments, the autoimmune disease or inflammatory disorder in the subject is scleroderma. In some embodiments, the autoimmune disease or inflammatory disorder in the subject is thyroiditis. In some embodiments, the autoimmune disease or inflammatory disorder in the subject is inflammatory arthritis. In some embodiments, the autoimmune disease or inflammatory disorder in the subject is diabetes mellitus. In some embodiments, the autoimmune disease or inflammatory disorder in the subject is oculo-bulbar myasthenia gravis. In some embodiments, the autoimmune disease or inflammatory disorder in the subject is crescentic IgA glomerulonephritis. In some embodiments, the autoimmune disease or inflammatory disorder in the subject is cholecystitis. In some embodiments, the autoimmune disease or inflammatory disorder in the subject is cerebral vasculitis. In some embodiments, the autoimmune disease or inflammatory disorder in the subject is Stevens-Johnson syndrome. In some embodiments, the autoimmune disease or inflammatory disorder in the subject is bullous pemphigoid.

In some embodiments, administration of the IL-2 conjugate and the anti-PD-1 antibody or antigen-binding fragment thereof to the subject does not cause changes in mental status, speech difficulties, cortical blindness, limb or gait ataxia, hallucinations, agitation, obtundation, or coma in the subject. In some embodiments, administration of the IL-2 conjugate and the anti-PD-1 antibody or antigen-binding fragment thereof to the subject does not cause seizures in the subject. In some embodiments, administration of the IL-2 conjugate and the anti-PD-1 antibody or antigen-binding fragment thereof to the subject is not contraindicated in subjects having a known seizure disorder.

In some embodiments, administration of the IL-2 conjugate and the anti-PD-1 antibody or antigen-binding fragment thereof to the subject does not cause capillary leak syndrome in the subject. In some embodiments, administration of the IL-2 conjugate and the anti-PD-1 antibody or antigen-binding fragment thereof to the subject does not cause Grade 2, Grade 3, or Grade 4 capillary leak syndrome in the subject. In some embodiments, administration of the IL-2 conjugate and the anti-PD-1 antibody or antigen-binding fragment thereof to the subject does not cause Grade 2 capillary leak syndrome in the subject. In some embodiments, administration of the IL-2 conjugate and the anti-PD-1 antibody or antigen-binding fragment thereof to the subject does not cause Grade 3 capillary leak syndrome in the subject. In some embodiments, administration of the IL-2 conjugate and the anti-PD-1 antibody or antigen-binding fragment thereof to the subject does not cause Grade 4 capillary leak syndrome in the subject.

In some embodiments, administration of the IL-2 conjugate and the anti-PD-1 antibody or antigen-binding fragment thereof to the subject does not cause a drop in mean arterial blood pressure in the subject following administration. In some embodiments, administration of the IL-2 conjugate and the anti-PD-1 antibody or antigen-binding fragment thereof to the subject does cause hypotension in the subject. In some embodiments, administration of the IL-2 conjugate and the anti-PD-1 antibody or antigen-binding fragment thereof to the subject does not cause the subject to experience a systolic blood pressure below 90 mm Hg or a 20 mm Hg drop from baseline systolic pressure.

In some embodiments, administration of the IL-2 conjugate and the anti-PD-1 antibody or antigen-binding fragment thereof to the subject does not cause edema or impairment of kidney or liver function in the subject.

In some embodiments, administration of the IL-2 conjugate and the anti-PD-1 antibody or antigen-binding fragment thereof to the subject does not cause eosinophilia in the subject. In some embodiments, administration of the IL-2 conjugate and the anti-PD-1 antibody or antigen-binding fragment thereof to the subject does not cause the eosinophil count in the peripheral blood of the subject to exceed 500 per μL. In some embodiments, administration of the IL-2 conjugate and the anti-PD-1 antibody or antigen-binding fragment thereof to the subject does not cause the eosinophil count in the peripheral blood of the subject to exceed 500 μL to 1500 per μL. In some embodiments, administration of the IL-2 conjugate and the anti-PD-1 antibody or antigen-binding fragment thereof to the subject does not cause the eosinophil count in the peripheral blood of the subject to exceed 1500 per μL to 5000 per μL. In some embodiments, administration of the IL-2 conjugate and the anti-PD-1 antibody or antigen-binding fragment thereof to the subject does not cause the eosinophil count in the peripheral blood of the subject to exceed 5000 per μL. In some embodiments, administration of the IL-2 conjugate and the anti-PD-1 antibody or antigen-binding fragment thereof to the subject is not contraindicated in subjects on an existing regimen of psychotropic drugs.

In some embodiments, administration of the IL-2 conjugate and the anti-PD-1 antibody or antigen-binding fragment thereof to the subject does not cause significant elevation of IL-5 levels. In some embodiments, administration of the IL-2 conjugate and the anti-PD-1 antibody or antigen-binding fragment thereof to the subject results in IL-5 levels that are at or below the lowest level of detection.

In some embodiments, administration of the IL-2 conjugate and the anti-PD-1 antibody or antigen-binding fragment thereof to the subject does not cause cumulative toxicity, end organic toxicity, QTc prolongation, or other cardiac toxicity.

In some embodiments, administration of the IL-2 conjugate and the anti-PD-1 antibody or antigen-binding fragment thereof to the subject does not cause adverse events (AEs) of fever, hypotension, or hypoxia that are correlated with IL-5/IL-6 cytokine elevation. In some embodiments, administration of the IL-2 conjugate and the anti-PD-1 antibody or antigen-binding fragment thereof to the subject causes related TEAEs mostly consisting of flu-like symptoms, nausea, or vomiting that are transient and resolved with accepted standard of care.

In some embodiments, administration of the IL-2 conjugate and the anti-PD-1 antibody or antigen-binding fragment thereof to the subject is not contraindicated in subjects on an existing regimen of nephrotoxic, myelotoxic, cardiotoxic, or hepatotoxic drugs. In some embodiments, administration of the IL-2 conjugate and the anti-PD-1 antibody or antigen-binding fragment thereof to the subject is not contraindicated in subjects on an existing regimen of aminoglycosides, cytotoxic chemotherapy, doxorubicin, methotrexate, or asparaginase. In some embodiments, administration of the IL-2 conjugate and the anti-PD-1 antibody or antigen-binding fragment thereof to the subject is not contraindicated in subjects receiving combination regimens containing antineoplastic agents. In some embodiments, the antineoplastic agent is selected from dacarbazine, cis-platinum, tamoxifen and interferon-alpha.

In some embodiments, administration of the IL-2 conjugate and the anti-PD-1 antibody or antigen-binding fragment thereof to the subject does not cause one or more Grade 4 adverse events in the subject following administration. In some embodiments, Grade 4 adverse events are selected from hypothermia; shock; bradycardia; ventricular extrasystoles; myocardial ischemia; syncope; hemorrhage; atrial arrhythmia; phlebitis; AV block second degree; endocarditis; pericardial effusion; peripheral gangrene; thrombosis; coronary artery disorder; stomatitis; nausea and vomiting; liver function tests abnormal; gastrointestinal hemorrhage; hematemesis; bloody diarrhea; gastrointestinal disorder; intestinal perforation; pancreatitis; anemia; leukopenia; leukocytosis; hypocalcemia; alkaline phosphatase increase; blood urea nitrogen (BUN) increase; hyperuricemia; non-protein nitrogen (NPN) increase; respiratory acidosis; somnolence; agitation; neuropathy; paranoid reaction; convulsion; grand mal convulsion; delirium; asthma, lung edema; hyperventilation; hypoxia; hemoptysis; hypoventilation; pneumothorax; mydriasis; pupillary disorder; kidney function abnormal; kidney failure; and acute tubular necrosis. In some embodiments, administration of the IL-2 conjugate and the anti-PD-1 antibody or antigen-binding fragment thereof to a group of subjects does not cause one or more Grade 4 adverse events in greater than 1% of the subjects following administration. In some embodiments, Grade 4 adverse events are selected from hypothermia; shock; bradycardia; ventricular extrasystoles; myocardial ischemia; syncope; hemorrhage; atrial arrhythmia; phlebitis; AV block second degree; endocarditis; pericardial effusion; peripheral gangrene; thrombosis; coronary artery disorder; stomatitis; nausea and vomiting; liver function tests abnormal; gastrointestinal hemorrhage; hematemesis; bloody diarrhea; gastrointestinal disorder; intestinal perforation; pancreatitis; anemia; leukopenia; leukocytosis; hypocalcemia; alkaline phosphatase increase; blood urea nitrogen (BUN) increase; hyperuricemia; non-protein nitrogen (NPN) increase; respiratory acidosis; somnolence; agitation; neuropathy; paranoid reaction; convulsion; grand mal convulsion; delirium; asthma, lung edema; hyperventilation; hypoxia; hemoptysis; hypoventilation; pneumothorax; mydriasis; pupillary disorder; kidney function abnormal; kidney failure; and acute tubular necrosis.

In some embodiments, administration of the IL-2 conjugate and the anti-PD-1 antibody or antigen-binding fragment thereof to a group of subjects does not cause one or more adverse events in greater than 1% of the subjects following administration, wherein the one or more adverse events is selected from duodenal ulceration; bowel necrosis; myocarditis; supraventricular tachycardia; permanent or transient blindness secondary to optic neuritis; transient ischemic attacks; meningitis; cerebral edema; pericarditis; allergic interstitial nephritis; and tracheo-esophageal fistula.

In some embodiments, administration of the IL-2 conjugate and the anti-PD-1 antibody or antigen-binding fragment thereof to a group of subjects does not cause one or more adverse events in greater than 1% of the subjects following administration, wherein the one or more adverse events is selected from malignant hyperthermia; cardiac arrest; myocardial infarction; pulmonary emboli; stroke; intestinal perforation; liver or renal failure; severe depression leading to suicide; pulmonary edema; respiratory arrest; respiratory failure.

In some embodiments, administration of the IL-2 conjugate and the anti-PD-1 antibody or antigen-binding fragment thereof to the subject stimulates CD8+ cells in a subject. In some embodiments, administration of the IL-2 conjugate and the anti-PD-1 antibody or antigen-binding fragment thereof to the subject stimulates NK cells in a subject. Stimulation may comprise an increase in the number of CD8+ cells in the subject, e.g., about 4, 5, 6, or 7 days after administration, or about 1, 2, 3, or 4 weeks after administration. In some embodiments, the CD8+ cells comprise memory CD8+ cells. In some embodiments, the CD8+ cells comprise effector CD8+ cells. Stimulation may comprise an increase in the proportion of CD8+ cells that are Ki67 positive in the subject, e.g., about 4, 5, 6, or 7 days after administration, or about 1, 2, 3, or 4 weeks after administration. Stimulation may comprise an increase in the number of NK cells in the subject, e.g., about 4, 5, 6, or 7 days after administration, or about 1, 2, 3, or 4 weeks after administration.

In some embodiments, CD8+ cells are expanded in the subject following administration of the IL-2 conjugate and the anti-PD-1 antibody or antigen-binding fragment thereof by at least 1.5-fold, such as by at least 1.6-fold, 1.7-fold, 1.8-fold, or 1.9-fold. In some embodiments, NK cells are expanded in the subject following administration of the IL-2 conjugate and the anti-PD-1 antibody or antigen-binding fragment thereof by at least 5-fold, such as by at least 5.5-fold, 6-fold, or 6.5-fold. In some embodiments, eosinophils are expanded in the subject following administration of the IL-2 conjugate and the anti-PD-1 antibody or antigen-binding fragment thereof by no more than about 2-fold, such as no more than about 1.5-fold, 1.4-fold, or 1.3-fold. In some embodiments, CD4+ cells are expanded in the subject following administration of the IL-2 conjugate and the anti-PD-1 antibody or antigen-binding fragment thereof by no more than about 2-fold, such as no more than about 1.8-fold, 1.7-fold, or 1.6-fold. In some embodiments, the expansion of CD8+ cells and/or NK cells in the subject following administration of the IL-2 conjugate and the anti-PD-1 antibody or antigen-binding fragment thereof is greater than the expansion of CD4+ cells and/or eosinophils. In some embodiments, the expansion of CD8+ cells is greater than the expansion of CD4+ cells. In some embodiments, the expansion of NK cells is greater than the expansion of CD4+ cells. In some embodiments, the expansion of CD8+ cells is greater than the expansion of eosinophils. In some embodiments, the expansion of NK cells is greater than the expansion of eosinophils. Fold expansion is determined relative to a baseline value measured before administration of the IL-2 conjugate. In some embodiments, fold expansion is determined at any of the times after administration, such as about 4, 5, 6, or 7 days after administration, or about 1, 2, 3, or 4 weeks after administration.

In some embodiments, administration of the IL-2 conjugate and the anti-PD-1 antibody or antigen-binding fragment thereof to the subject increases the number of peripheral CD8+T and NK cells in the subject without increasing the number of peripheral CD4+ regulatory T cells in the subject. In some embodiments, administration of the IL-2 conjugate and the anti-PD-1 antibody or antigen-binding fragment thereof to the subject increases the number of peripheral CD8+T and NK cells in the subject without increasing the number of peripheral eosinophils in the subject. In some embodiments, administration of the IL-2 conjugate and the anti-PD-1 antibody or antigen-binding fragment thereof to the subject increases the number of peripheral CD8+T and NK cells in the subject without increasing the number of intratumoral CD8+T and NK cells in the subject and without increasing the number of intratumoral CD4+ regulatory T cells in the subject.

In some embodiments, administration of the IL-2 conjugate and the anti-PD-1 antibody or antigen-binding fragment thereof to the subject does not require the availability of an intensive care facility or skilled specialists in cardiopulmonary or intensive care medicine. In some embodiments, administration of the IL-2 conjugate and the anti-PD-1 antibody or antigen-binding fragment thereof to the subject does not require the availability of an intensive care facility or skilled specialists in cardiopulmonary or intensive care medicine. In some embodiments, administration of the IL-2 conjugate and the anti-PD-1 antibody or antigen-binding fragment thereof to the subject does not require the availability of an intensive care facility. In some embodiments, administration of the IL-2 conjugate and the anti-PD-1 antibody or antigen-binding fragment thereof to the subject does not require the availability of skilled specialists in cardiopulmonary or intensive care medicine.

In some embodiments, administration of the IL-2 conjugate and the anti-PD-1 antibody or antigen-binding fragment thereof does not cause dose-limiting toxicity. In some embodiments, administration of the IL-2 conjugate and the anti-PD-1 antibody or antigen-binding fragment thereof does not cause severe cytokine release syndrome. In some embodiments, the IL-2 conjugate does not induce anti-drug antibodies (ADAs), i.e., antibodies against the IL-2 conjugate. In some embodiments, a lack of induction of ADAs is determined by direct immunoassay for antibodies against PEG and/or ELISA for antibodies against the IL-2 conjugate. An IL-2 conjugate is considered not to induce ADAs if a measured level of ADAs is statistically indistinguishable from a baseline (pre-treatment) level or from a level in an untreated control.

### Additional Agents

In some embodiments, the methods further comprise administering to the subject a therapeutically effective amount of one or more chemotherapeutic agents, in addition to the anti-PD-1 antibody or antigen-binding fragment thereof. In some embodiments, the one or more chemotherapeutic agents comprises one or more platinum-based chemotherapeutic agents. In some embodiments, the one or more chemotherapeutic agents comprises carboplatin and pemetrexed. In some embodiments, the one or more chemotherapeutic agents comprises carboplatin and nab-paclitaxel. In some embodiments, the one or more chemotherapeutic agents comprises carboplatin and docetaxel. In some embodiments, the cancer in the subject is non-small cell lung cancer (NSCLC).

### Kits/Article of Manufacture

Disclosed herein, in certain embodiments, are kits and articles of manufacture for use with one or more methods and compositions described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself, a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for drugs, or the approved product insert. In one embodiment, compositions containing a compound provided herein formulated in a compatible pharmaceutical carrier are also prepared, placed in an appropriate container, and labeled for treatment of an indicated condition.

In certain embodiments, the anti-PD-1 antibody or antigen-binding fragment thereof provided by the kit may be pembrolizumab formulated as a liquid medicament which comprises 25 mg/ml pembrolizumab, 7% (w/v) sucrose, 0.02% (w/v) polysorbate 80 in 10 mM histidine buffer pH 5.5.

### EXAMPLES

These examples are provided for illustrative purposes only and not to limit the scope of the claims provided herein.

### Example 1. Preparation of Pegylated IL-2 Conjugates

An exemplary method with details for preparing IL-2 conjugates described herein is provided in this Example.

IL-2 employed for bioconjugation was expressed as inclusion bodies in E. coli using methods disclosed herein, using: (a) an expression plasmid encoding (i) the protein with the desired amino acid sequence, which gene contains a first unnatural base pair to provide a codon at the desired position at which an unnatural amino acid N6-((2-azidoethoxy)-carbonyl)-L-lysine (AzK) was incorporated and (ii) a tRNA derived from M. mazei Pyl, which gene comprises a second unnatural nucleotide to provide a matching anticodon in place of its native sequence; (b) a plasmid encoding a M. barkeri derived pyrrolysyl-tRNA synthetase (Mb PylRS), (c) N6-((2-azidoethoxy)-carbonyl)-L-lysine (AzK); and (d) a truncated variant of nucleotide triphosphate transporter PtNTT2 in which the first 65 amino acid residues of the full-length protein were deleted. The double-stranded oligonucleotide that encodes the amino acid sequence of the desired IL-2 variant contained a codon AXC as codon 64 of the sequence that encodes the protein having SEQ ID NO: 1 in which P64 is replaced with an unnatural amino acid described herein. The plasmid encoding an orthogonal tRNA gene from M. mazei comprised an AXC-matching anticodon GYT in place of its native sequence, wherein Y is an unnatural nucleotide as disclosed herein. X and Y were selected from unnatural nucleotides dTPT3 and dNaM as disclosed herein. The expressed protein was extracted from inclusion bodies and re-folded using standard procedures before site-specifically pegylating the AzK-containing IL-2 product using DBCO-mediated copper-free click chemistry to attach stable, covalent mPEG moieties to the AzK. Examplary reactions are shown in Schemes 1 and 2 (wherein n indicates the number of repeating PEG units). The reaction of the AzK moiety with the DBCO alkynyl moiety may afford one regioisomeric product or a mixture of regioisomeric products.

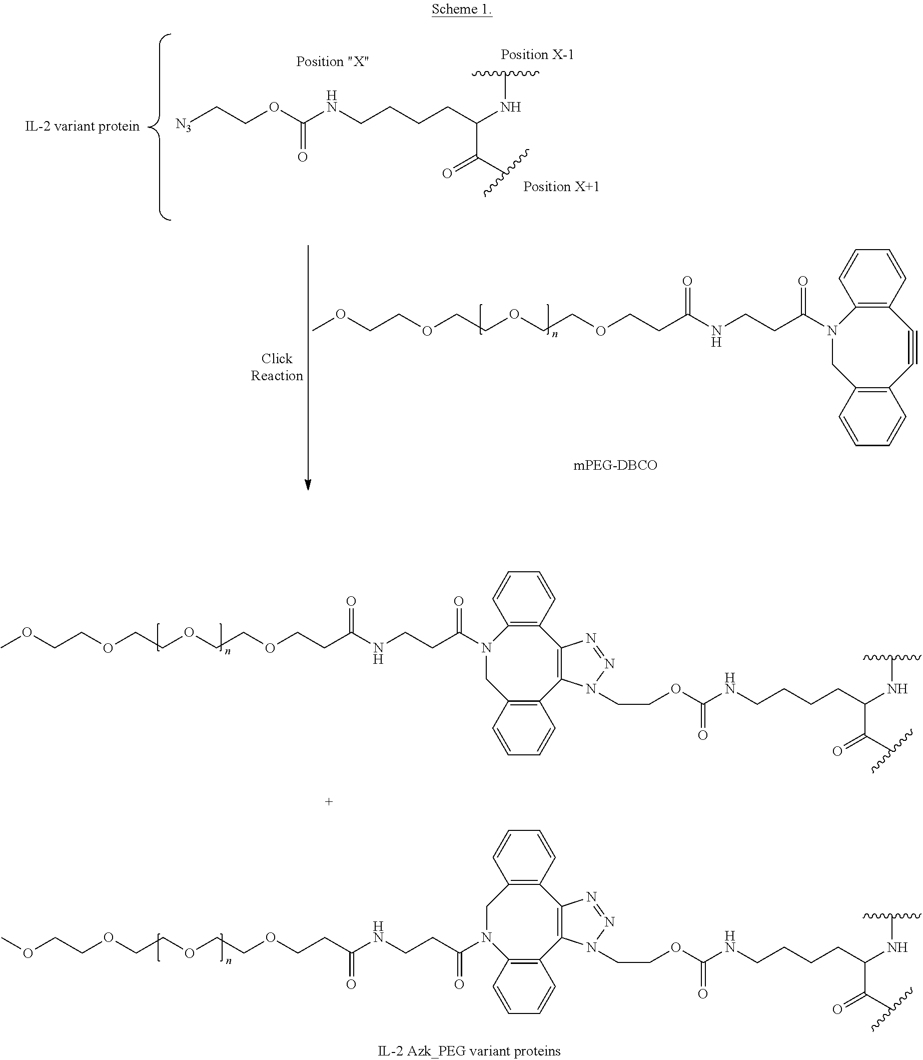

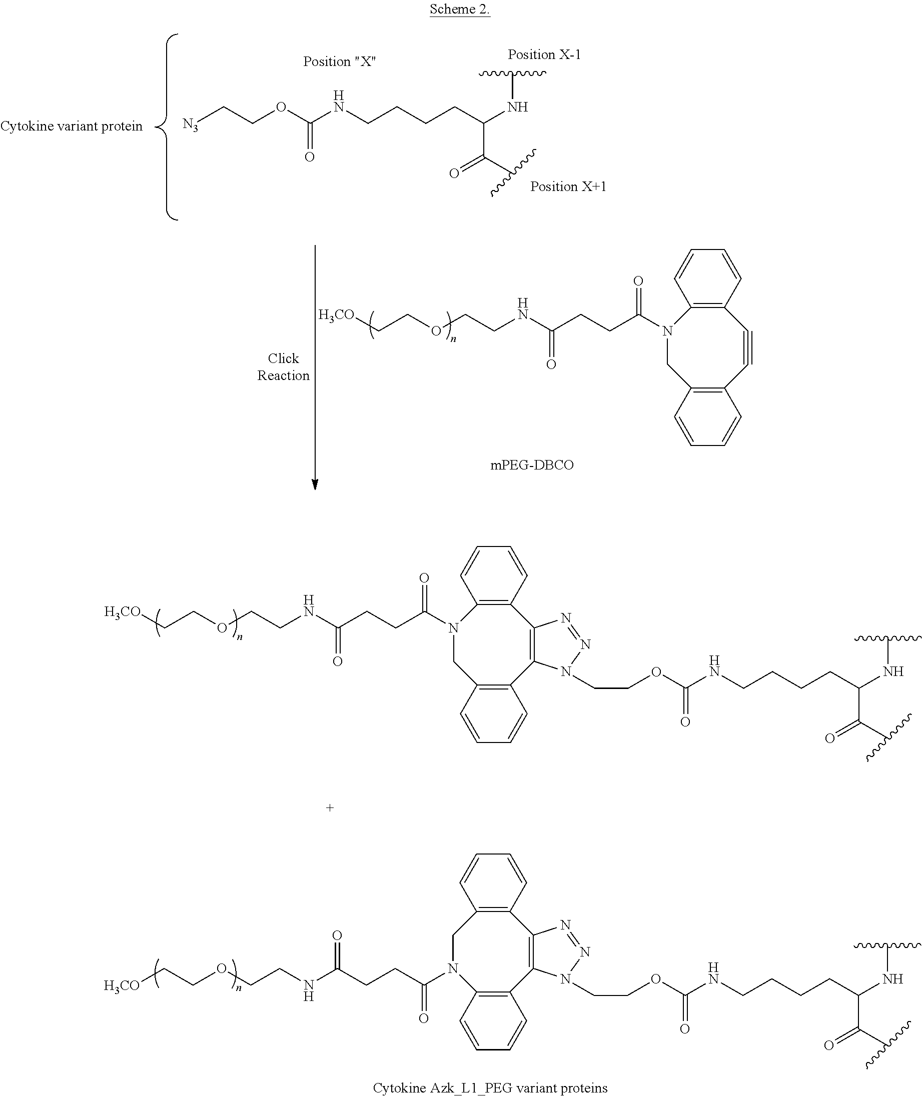

### Example 2. Clinical Study of Biomarker Effects Following IL-2 Conjugate and Pembrolizumab Administration

A study was performed to characterize immunological effects of in vivo administration of an IL-2 conjugate described herein in combination with pembrolizumab. The IL-2 conjugate comprised SEQ ID NO: 2, wherein position 64 is AzK_L1_PEG30kD, where AzK_L1_PEG30kD is defined as a structure of Formula (IV) or Formula (V), or a mixture of Formula (IV) and Formula (V), and a 30 kDa, linear mPEG chain. This IL-2 conjugate can also be described as an IL-2 conjugate comprising SEQ ID NO: 1, wherein position 64 is replaced by the structure of Formula (IV) or Formula (V), or a mixture of Formula (IV) and Formula (V), and a 30 kDa, linear mPEG chain. The IL-2 conjugate can also be described as an IL-2 conjugate comprising SEQ ID NO: 1, wherein position 64 is replaced by the structure of Formula (XII) or Formula (XIII), or a mixture of Formula (XII) and Formula (XIII), and a 30 kDa, linear mPEG chain. This IL-2 conjugate has a proposed International Nonproprietary Name (pINN) of pegenzileukin. The compound was prepared as described in Example 1, i.e., using methods wherein a protein was first prepared having SEQ ID NO: 1 in which the proline at position 64 was replaced by N6-((2-azidoethoxy)-carbonyl)-L-lysine AzK. The AzK-containing protein was then allowed to react under click chemistry conditions with DBCO comprising a methoxy, linear PEG group having an average molecular weight of 30 kDa, followed by purification and formulation employing standard procedures.

The IL-2 conjugate and pembrolizumab were administered via IV infusion for 30 minutes every 3 weeks [Q3W]. Effects on the following biomarkers were analyzed as surrogate predictors of safety and/or efficacy:
Eosinophilia (elevated peripheral eosinophil count): Cell surrogate marker for IL-2-induced proliferation of cells (eosinophils) linked to vascular leak syndrome (VLS);
Interleukin 5 (IL-5): Cytokine surrogate marker for IL-2 induced activation of type 2 innate lymphoid cells and release of this chemoattractant that leads to eosinophilia and potentially VLS;
Interleukin 6 (IL-6): Cytokine surrogate marker for IL-2 induced cytokine release syndrome (CRS); and
Interferon γ (IFN-γ): Cytokine surrogate marker for IL-2 induced activation of CD8+ cytotoxic T lymphocytes and NK cells.

Effects on the cell counts of the following biomarkers were analyzed as surrogate predictors of anti-tumor immune activity:
Peripheral CD8+ Effector Cells: Marker for IL-2-induced proliferation of these target cells in the periphery that upon infiltration become a surrogate marker of inducing a potentially latent therapeutic response;
Peripheral CD8+ Memory Cells: Marker for IL-2-induced proliferation of these target cells in the periphery that upon infiltration become a surrogate marker of inducing a potentially durable latent therapeutic and maintenance of the memory population;
Peripheral NK Cells: Marker for IL-2-induced proliferation of these target cells in the periphery that upon infiltration become a surrogate marker of inducing a potentially rapid therapeutic response; and
Peripheral CD4+ Regulatory Cells: Marker for IL-2-induced proliferation of these target cells in the periphery that upon infiltration become a surrogate marker of inducing an immunosuppressive TME and offsetting of an effector-based therapeutic effect.

Subjects were human males or females aged≥18 years at screening. All subjects had been previously treated with an anti-cancer therapy and met at least one of the following: Treatment related toxicity resolved to grade 0 or 1 (alopecia excepted) according to NCI CTCAE v5.0; or Treatment related toxicity resolved to at least grade 2 according to NCI CTCAE v5.0 with prior approval of the Medical Monitor. The most common tumors included cervical cancer, head and neck squamous cell carcinoma, basal cell carcinoma, melanoma and non-small cell lung cancer.

Subjects also met the following criteria: Provided informed consent. Eastern Cooperative Oncology Group (ECOG) performance status of 0 or 1. Life expectancy greater than or equal to 12 weeks as determined by the Investigator. Histologically or cytologically confirmed diagnosis of advanced and/or metastatic solid tumors. Subjects with advanced or metastatic solid tumors who have refused standard of care; or for whom no reasonable standard of care exists that would confer clinical benefit; or for whom standard therapy is intolerable, not effective, or not accessible. Measurable disease per RECIST v1.1. Adequate laboratory parameters including: Absolute lymphocyte count≥0.5 times lower limit of normal; Platelet count≥100×10⁹/L; Hemoglobin≥9.0 g/dL (absence of growth factors or transfusions within 2 weeks; 1-week washout for ESA and CSF administration is sufficient); Absolute neutrophil count≥1.5×10⁹/L (absence of growth factors within 2 weeks); Prothrombin time (PT) and partial thromboplastin time (PTT)≤1.5 times upper limit of normal (ULN); Aspartate aminotransferase (AST) and alanine aminotransferase (ALT)≤2.5 times ULN except if liver metastases are present may be ≤5 times ULN; Total bilirubin≤1.5 x ULN. Premenopausal women and women less than 12 months after menopause had a negative serum pregnancy test within 7 days prior to initiating study treatment.

Cohorts Treated with 8 μg/Kg and 16 μg/Kg Doses

Q3W dosing. 14 adults (9 [64.3%] male, 5 [35.7%] female, 9 [64.2%] Caucasian) having advanced or metastatic solid tumors and whose age ranged from 29-74 years received a) the IL-2 conjugate at an 8 μg/kg dose IV Q3W or 16 μg/kg dose IV Q3W and b) pembrolizumab at a dose of 200 mg IV Q3W sequentially for at least one cycle. Here and throughout Example 2, drug mass per kg subject (e.g., 8 μg/kg) refers to IL-2 mass exclusive of PEG and linker mass. The results below are for subjects receiving an 8 μg/kg dose IV Q3W and pembrolizumab (4 subjects) or 16 μg/kg dose IV Q3W and pembrolizumab (10 subjects), who received treatment for 2-19 cycles.

Two subjects who received 8 μg/kg IL-2 conjugate and pembrolizumab had confirmed partial responses (PRs; 1 PD-1-naïve basal cell carcinoma, 1 head and neck squamous cell carcinoma, who had received prior anti-PD-1) ongoing for 22+ months. One subject (non-small cell lung cancer) who received 16 μg/kg IL-2 conjugate and pembrolizumab had disease stabilization for about 6 months. Six subjects had disease progression (at the 6-week assessment); one subject had initial disease stabilization (at the 6 week assessment; followed by progressive disease). The four subjects receiving 8 μg/kg IL-2 conjugate and pembrolizumab had increased post-dose CD8+Ki67 expression levels (15%-70%).

One 59 year old male with head and neck squamous cell carcinoma receiving 8 μg/kg IL-2 conjugate and pembrolizumab received 30+ cycles and had a confirmed partial response (39% decrease after 8 cycles; 47% decrease after 11 cycles). This subject had previously received 4 lines of systemic therapy including 2 anti-PD1 treatments; the best response to an anti-PD1 treatment had been stable disease.

One 50 year old male with basal cell carcinoma receiving 8 μg/kg IL-2 conjugate and pembrolizumab received 30 cycles and had a confirmed partial response (50% decrease after 2 cycles, and 80% decrease after 8 cycles). This subject had previously undergone surgeries and radiation therapy.

The maximal tumor responses in other patients with immune sensitive tumors were found to be melanoma (23% and 11% growth), basal cell carcinoma (4% growth), and non-small cell lung cancer (29% reduction).

The peak peripheral expansion of CD8+T effector cells averaged 2.02-fold above baseline in subjects receiving 8 μg/kg IL-2 conjugate and pembrolizumab. All four subjects had post-dose NK Cell Ki67 expression levels of nearly 100 percent. The subjects had post-dose peak peripheral expansion of NK cells that averaged 6.73-fold above baseline at day 3. The peak peripheral expansion of CD8+T effector cells averaged 3.71-fold above baseline in subjects receiving 16 μg/kg IL-2 conjugate and pembrolizumab.

Efficacy biomarkers. Data relating to efficacy biomarkers was based on data available for 10 subjects (4 subjects receiving the IL-2 conjugate at 8 μg/kg; 6 subjects receiving the IL-2 conjugate at 16 μg/kg). Peripheral CD8+T_{eff} cell counts were measured (FIGS. 1A-C). Prolonged CD8+ expansion over baseline (e.g., greater than or equal to 1.5-fold change) was observed at 3 weeks after the previous dose in some subjects. The percentage of CD8+T_{eff} cells expressing Ki67 was also measured (FIG. 2).

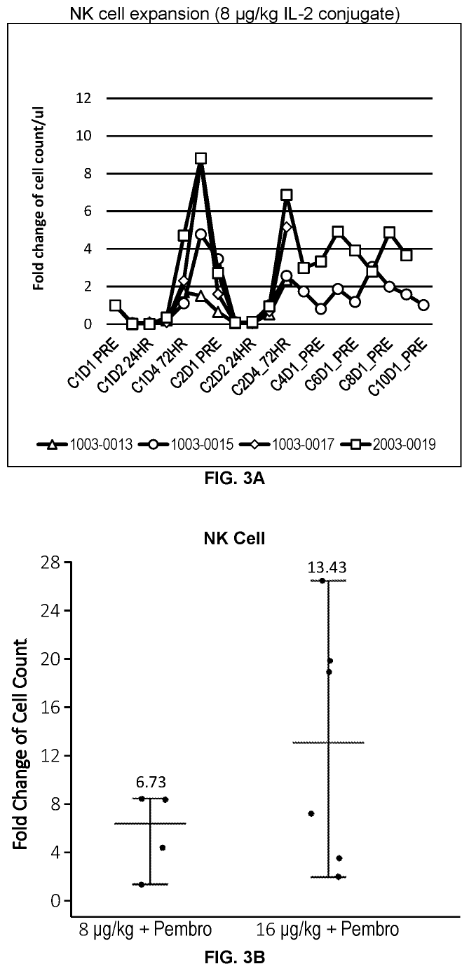

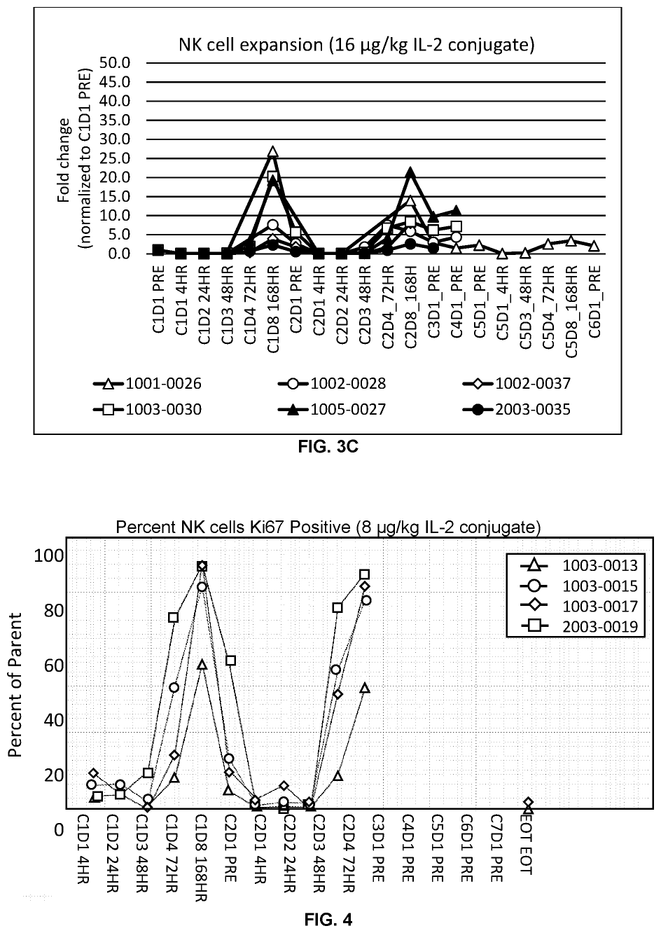

Peripheral NK cell counts are shown in FIGS. 3A-C. Prolonged NK cell expansion over baseline (e.g., greater than or equal to 2-fold change) was observed at 3 weeks after the previous dose in some subjects. The percentage of NK cells expressing Ki67 was also measured (FIG. 4).

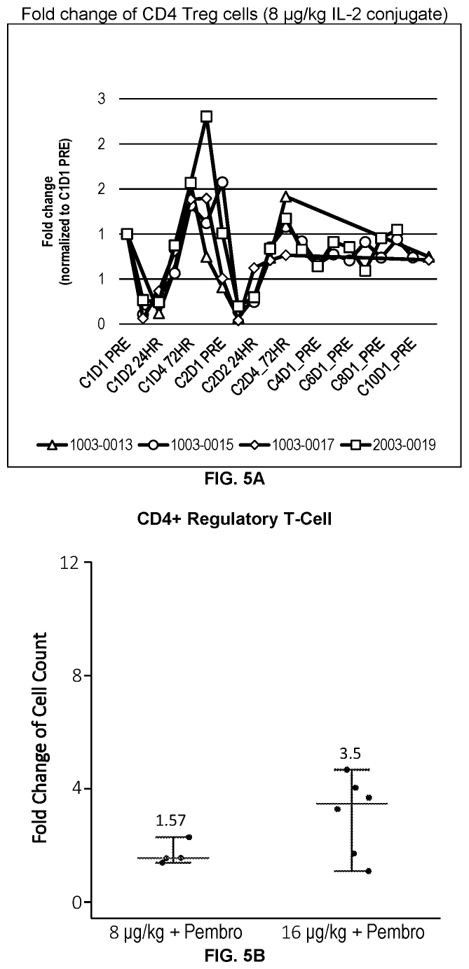

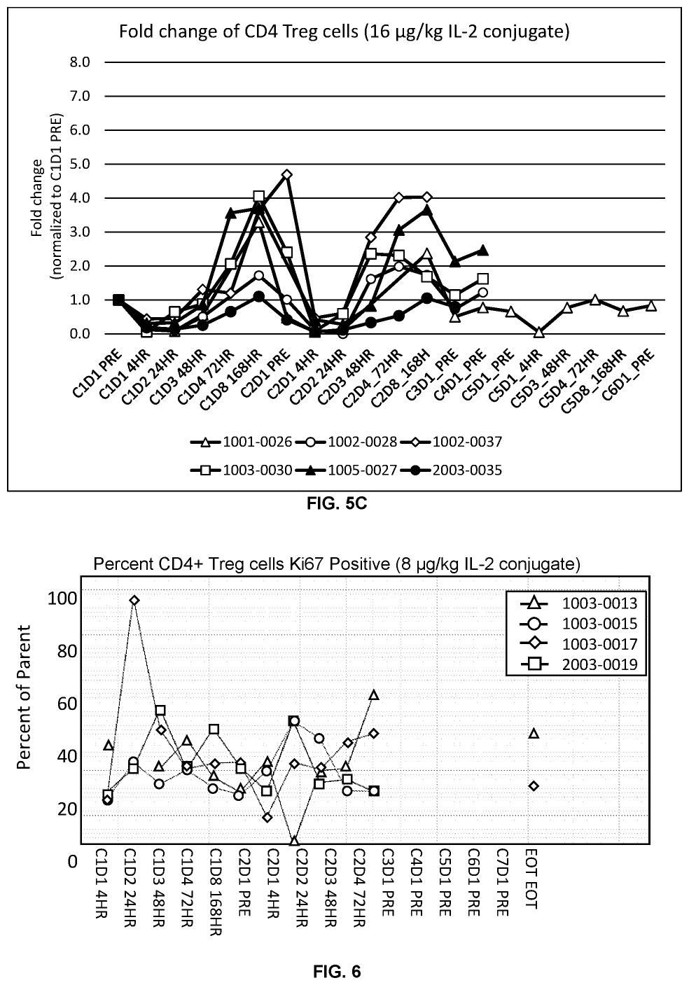

Peripheral CD4+ T_{reg} counts are shown in FIGS. 5A-C. The percentage of CD4+ T_{reg} cells expressing Ki67 was also measured (FIG. 6).

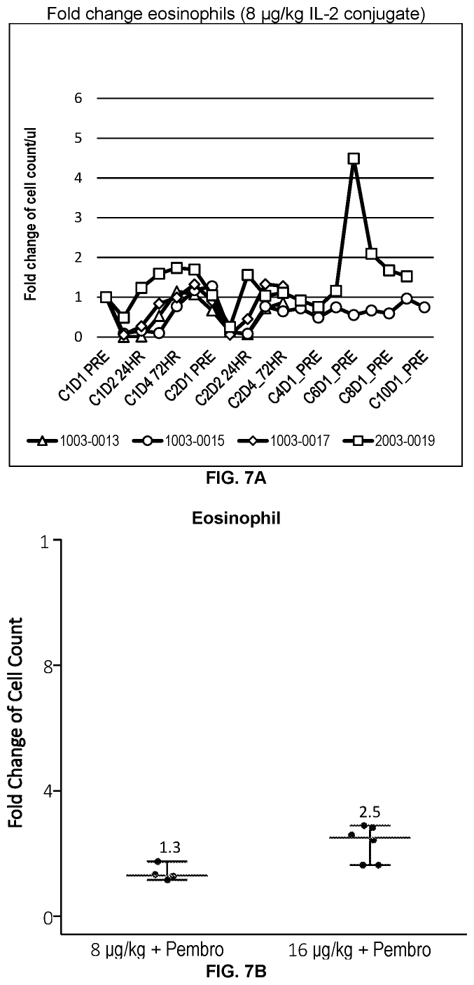

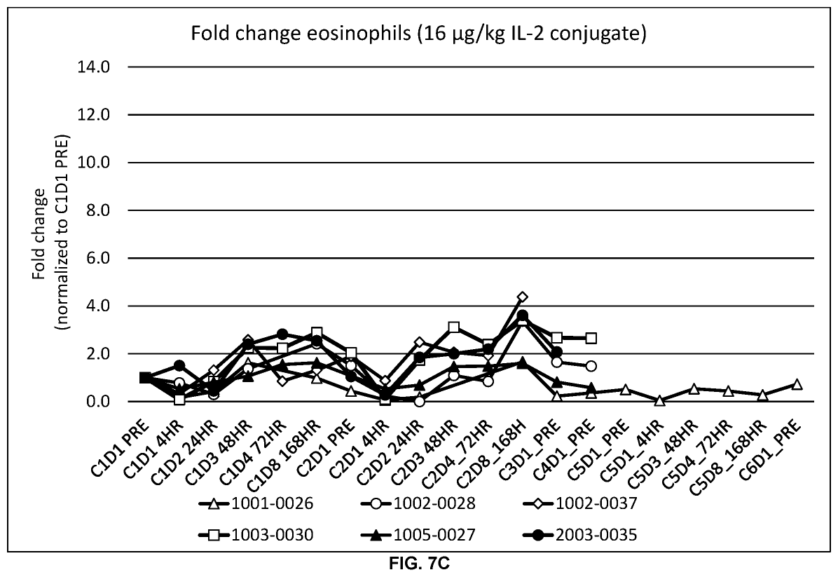

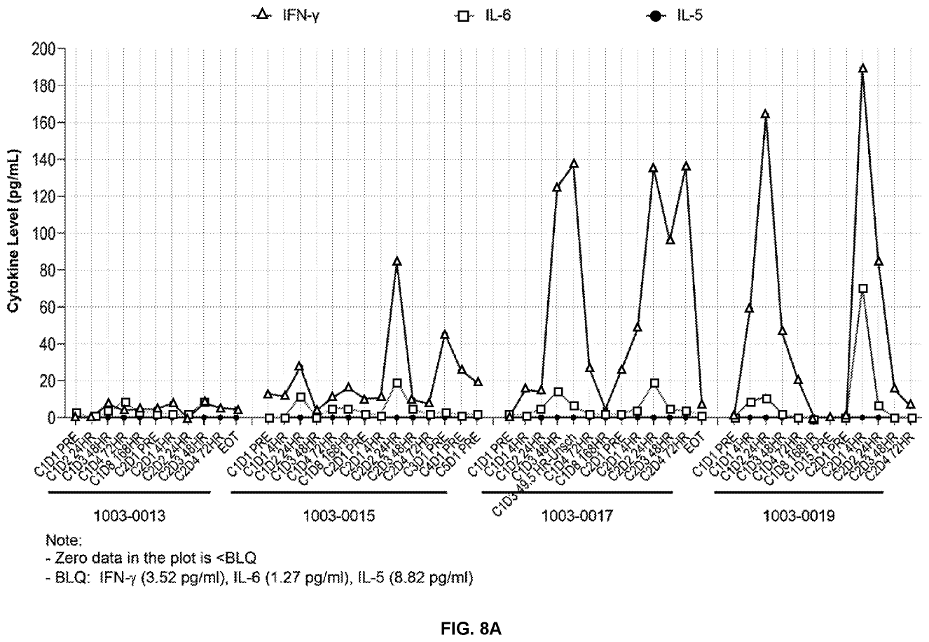

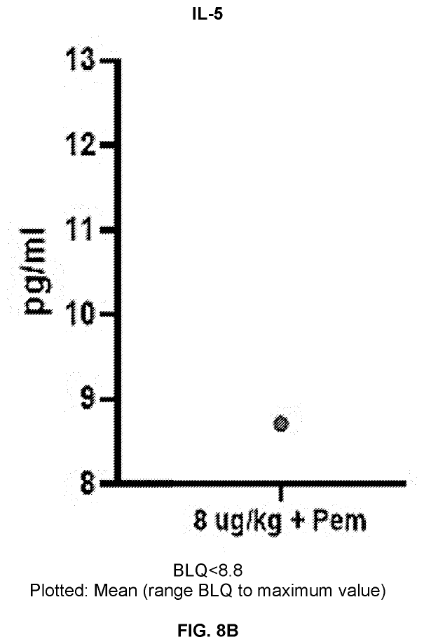

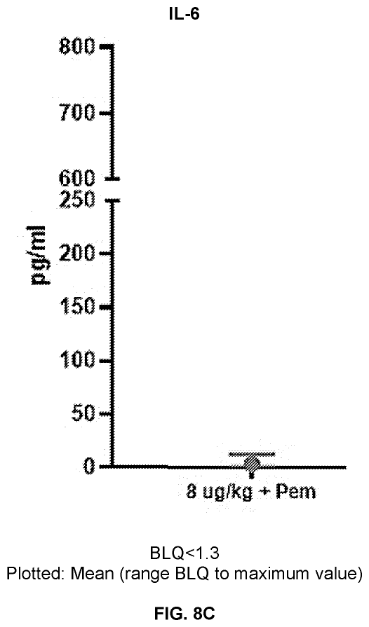

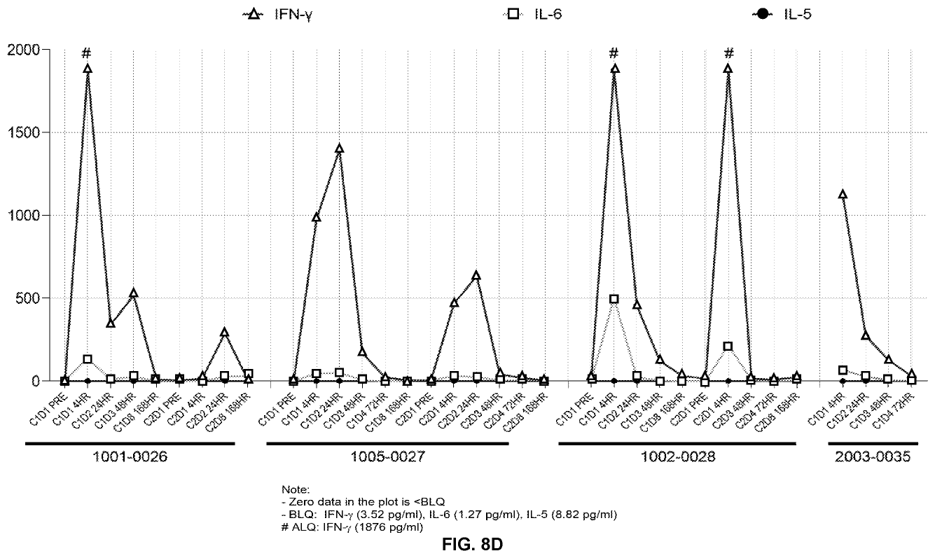

Eosinophil counts were measured (FIGS. 7A-C). The measured values were consistently below the range of 2328-15958 eosinophils/μL in patients with IL-2 induced eosinophilia as reported in Pisani et al., Blood 1991 Sep. 15; 78(6):1538-44. Levels of IFN-γ, IL-5, and IL-6 were also measured (FIGS. 8A-D). The measured values show that IFN-γ was induced, but low amounts of IL-5 and IL-6, cytokines associated with VLS and CRS, respectively, were induced.

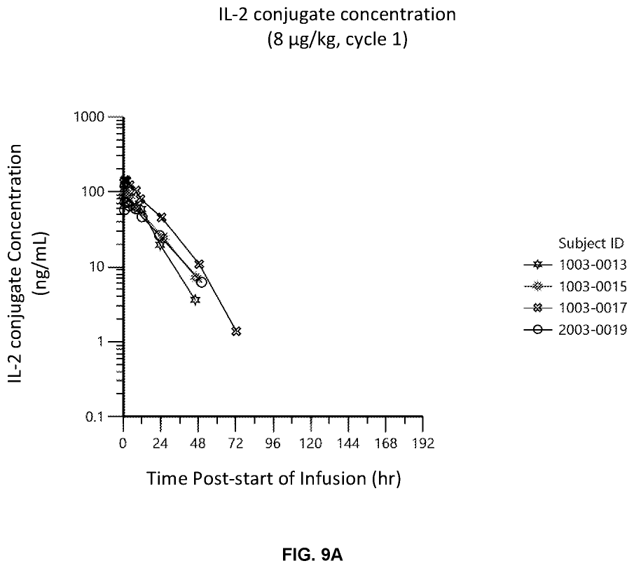

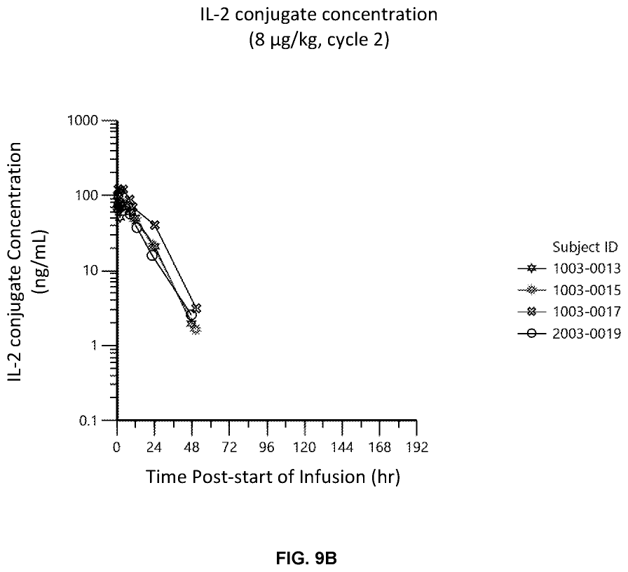

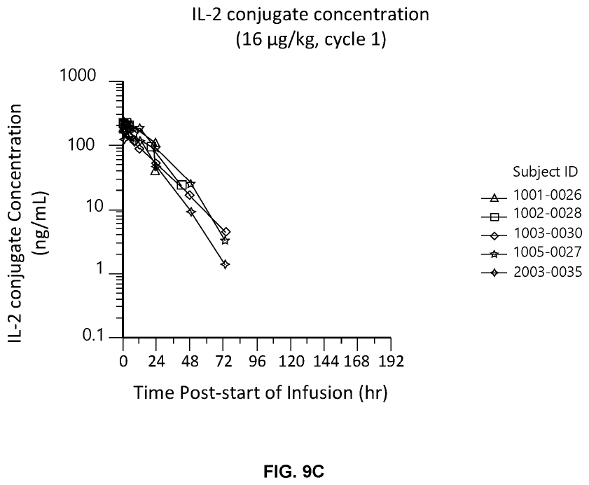

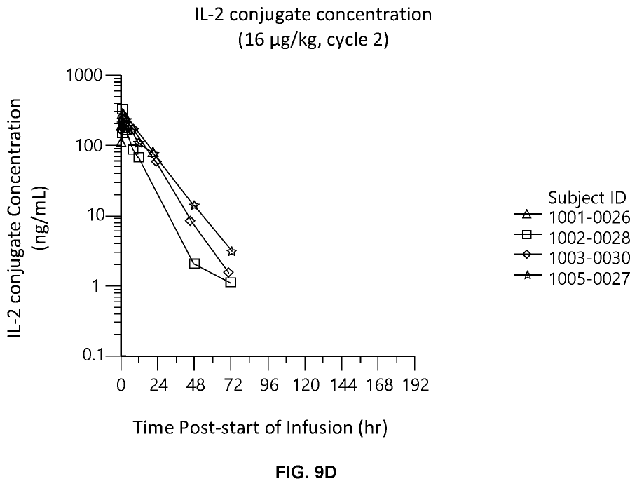

Mean concentrations of the IL-2 conjugate, administered at a dose of 8 μg/kg, after 1 and 2 cycles are shown in FIG. 9A and FIG. 9B, respectively. Mean concentrations of the IL-2 conjugate, administered at a dose of 16 μg/kg, after 1 and 2 cycles are shown in FIG. 9C and FIG. 9D, respectively.

Anti-drug Antibodies (ADAs). Samples from treated subjects were assayed after each dose cycle for anti-drug antibodies (ADAs). Anti-polyethylene glycol autoantibodies were detected by direct immunoassays (detection limit: 36 ng/mL). A bridging MesoScale Discovery ELISA was performed with a labeled form of the IL-2 conjugate, having a detection limit of 4.66 ng/mL. Additionally, a cell-based assay for neutralizing antibodies against the IL-2 conjugate was performed using the CTLL-2 cell line, with STAT5 phosphorylation as the readout (detection limit: 6.3 μg/mL).

Samples were collected and analyzed after each dose cycle from four subjects where 2 patients received 2 cycles and the other two patients received 10 or 11 cycles. An assay-specific cut point was determined during assay qualification as a signal to negative ratio of 1.09 or higher for the IL-2 conjugate ADA assay and 2.08 for the PEG ADA assay. Samples that gave positive or inconclusive results in the IL-2 conjugate assay were subjected to confirmatory testing in which samples and controls were assayed in the presence and absence of confirmatory buffer (10 μg/mL IL-2 conjugate in blocking solution). Samples that gave positive or inconclusive results in the PEG assay were subjected to confirmatory testing in which samples and controls were assayed in the presence and absence of confirmatory buffer (10 μg/mL IL-2 conjugate in 6% horse serum). Samples will be considered “confirmed” if their absorbance signal is inhibited by equal to or greater than an assay-specific cut point determined during assay qualification (14.5% for the IL-2 conjugate or 42.4% for PEG) in the detection step. No confirmed ADA against the IL-2 conjugate or PEG were detected (data not shown).

Summary of Results; Discussion. All subjects had elevated post-dose CD8+Ki67 expression levels (FIG. 2), with peripheral expansion of CD8+T effector (T_{eff}) cells averaging 1.95-fold above baseline. All 4 subjects also had elevated post-dose NK cell Ki67 expression levels (FIG. 4), with peripheral expansion of NK cells averaging 6.73-fold above baseline at day 3. There were no meaningful elevations in IL-5 and IL-6 levels.

An AE was any untoward medical occurrence in a clinical investigation subject administered a pharmaceutical product, regardless of causal attribution. Dose-limiting toxicities were defined as an AE occurring within Day 1 through Day 29 (inclusive)+1 day of a treatment cycle that was not clearly or incontrovertibly solely related to an extraneous cause and that met at least one of the following criteria:
Grade 3 neutropenia (absolute neutrophil count≤1000/mm³>500/mm³) lasting≥7 days, or Grade 4 neutropenia of any duration
Grade 3+ febrile neutropenia
Grade 4+ thrombocytopenia (platelet count<25,000/mm³)
Grade 3+ thrombocytopenia (platelet count<50,000-25,000/mm³) lasting≥5 days, or associated with clinically significant bleeding or requiring platelet transfusion
Failure to meet recovery criteria of an absolute neutrophil count of at least 1,000 cells/mm³ and a platelet count of at least 75,000 cells/mm³ within 10 days
Any other grade 4+ hematologic toxicity lasting≥5 days
Grade 3+ ALT or AST in combination with a bilirubin≥2 times ULN with no evidence of cholestasis or another cause such as viral infection or other drugs (i.e. Hy's law)
Grade 3 infusion-related reaction that occurs with premedication; Grade 4 infusion-related reaction
Grade 3 Vascular Leak Syndrome defined as hypotension associated with fluid retention and pulmonary edema
Grade 3+ anaphylaxis
Grade 3+ hypotension
Grade 3+AE that does not resolve to grade<2 within 7 days of starting accepted standard of care medical management
Grade 3+ cytokine release syndrome The following exceptions applied to non-hematologic AEs:
Grade 3 fatigue, nausea, vomiting, or diarrhea that resolves to grade≤2 with optimal medical management in ≤3 days
Grade 3 fever (as defined by >40° C. for ≤24 hours)
Grade 3 infusion-related reaction that occurs without premedication; subsequent doses should use premedication and if reaction recurs then it will be a DLT
Grade 3 arthralgia or rash that resolves to grade≤2 within 7 days of starting accepted standard of care medical management (e.g. systemic corticosteroid therapy)
   If a subject had grade 1 or 2 ALT or AST elevation at baseline considered secondhand to liver metastases, a grade 3 elevation must also be ≥3 times baseline and last>7 days.

Serious AEs were defined as any AE that results in any of the following outcomes: Death; Life-threatening AE; Inpatient hospitalization or prolongation of an existing hospitalization; A persistent or significant incapacity or substantial disruption of the ability to conduct normal life functions; or a congenital anomaly/birth defect. Important medical events that may not result in death, be life-threatening, or require hospitalization may be considered serious when, based upon appropriate medical judgment, they may jeopardize the subject and may require medical or surgical intervention to prevent one of the outcomes listed above. Examples of such medical events include allergic bronchospasm requiring intensive treatment in an emergency room or at home, blood dyscrasias or convulsions that do not result in inpatient hospitalization, or the development of drug dependency or drug abuse.

There were no dose-limiting toxicities reported at either dose and there were no treatment-related adverse events (TRAE) leading to discontinuation. One TRAE led to dosage reduction. There were 5 treatment-related serious AEs reported in three of the patients treated at 16 μg/kg dose IV Q3W.

The most common TRAEs (>2 patients) of all grades by SOC included general disorders and administration conditions, investigations, metabolism and nutrition, nervous system disorders, respiratory, thoracic and mediastinal disorders, vascular disorders, skin and subcutaneous disorders, blood and lymphatic disorders, cardiac disorders, gastrointestinal disorders, immune system disorders, infections and infestations, and musculoskeletal. TEAEs by preferred terms are detailed in Table 3.

TABLE 3

| | 8 μg/kg IL-2 | | 16 μg/kg IL-2 | |
|---|---|---|---|---|
| | conjugate Q3W + | | conjugate Q3W + | |
| | pembrolizumab | | pembrolizumab | |
| | (N = 4) | | (N = 10) | |
| Primary system organ class | All | Grade | All | Grade |
| Preferred Term n (%) | Grades | ≥3 | Grades | ≥3 |
| Number of Participants | 3 (75.0) | 0 | 10 (100) | 6 (60.0) |
| with TEAE | | | | |
| INFECTIONS AND | 1 (25.0) | 0 | 1 (10.0) | 0 |
| INFESTATIONS | | | | |
| BLOOD AND LYMPHATIC | 0 | 0 | 2 (20.0) | 1 (10.0) |
| SYSTEM DISORDERS | | | | |
| IMMUNE SYSTEM | 0 | 0 | 2 (20.0) | 1 (10.0) |
| DISORDERS | | | | |
| ENDOCRINE DISORDERS | 0 | 0 | 2 (20.0) | 0 |
| METABOLISM AND | 1 (25.0) | 0 | 4 (40.0) | 1 (10.0) |
| NUTRITION DISORDERS | | | | |
| PSYCHIATRIC DISORDERS | 1 (25.0) | 0 | 0 | 0 |
| NERVOUS SYSTEM | 2 (50.0) | 0 | 3 (30.0) | 0 |
| DISORDERS | | | | |
| EYE DISORDERS | 1 (25.0) | 0 | 0 | 0 |
| CARDIAC DISORDERS | 0 | 0 | 2 (20.0) | 0 |
| VASCULAR DISORDERS | 1 (25.0) | 0 | 3 (30.0) | 1 (10.0) |
| RESPIRATORY, | 1 (25.0) | 0 | 3 (30.0) | 1 (10.0) |
| THORACIC AND | | | | |
| MEDIASTINAL DISORDERS | | | | |
| GASTROINTESTINAL | 1 (25.0) | 0 | 4 (40.0) | 0 |
| DISORDERS | | | | |
| HEPATOBILIARY | 0 | 0 | 1 (10.0) | 0 |
| DISORDERS | | | | |
| SKIN AND SUBCUTANEOUS | 1 (25.0) | 0 | 3 (30.0) | 0 |
| TISSUE DISORDERS | | | | |
| MUSCULOSKELETAL AND | 1 (25.0) | 0 | 3 (30.0) | 0 |
| CONNECTIVE TISSUE | | | | |
| DISORDERS | | | | |
| GENERAL DISORDERS AND | 3 (75.0) | 0 | 10 (100) | 0 |
| ADMINISTRATION SITE | | | | |
| CONDITIONS | | | | |
| INVESTIGATIONS | 2 (50.0) | 0 | 8 (80.0) | 4 (40.0) |
| INJURY, POISONING AND | 1 (25.0) | 0 | 1 (10.0) | 0 |
| PROCEDURAL | | | | |
| COMPLICATIONS | | | | |

Treatment-related AEs were transient and resolved with accepted standard of care. AEs of fever, hypotension, and hypoxia did not correlate with IL-5/IL-6 cytokine elevation. No cumulative toxicity, end organ toxicity, vascular leak syndrome, or eosinophilia was observed. IL-5 levels remained at or below the lowest level of detection. One subject had G2 hypotension which resolved with hydration. One subject had G3 cytokine release syndrome (fever+ hypotension requiring pressors; subject had baseline orthostatic hypotension) and came off therapy for progression. One subject developed recurrent G2 cytokine release syndrome with fever and hypoxia (patient had underlying COPD managed with supportive care including one dose of tociluzimab with resolution). The subject's dose was reduced to 8 μg/kg; the subject then developed G2 pneumonitis, and was rechallenged following improvement to G1. Subsequently, the subject developed recurrent G3 pneumonitis and did not receive further therapy. Otherwise, there was no notable impact to vital signs, no QTc prolongation or other cardiac toxicity, and no discontinuations due to TRAE. Accordingly, the IL-2 conjugate in combination with pembrolizumab demonstrated encouraging PD data and was generally well-tolerated. It was determined that the in vivo half-life of the IL-2 conjugate was about 10 hours. Overall, the results are considered to support non-alpha preferential activity of the IL-2 conjugate, with a tolerable safety profile in combination with pembrolizumab as well as encouraging PD and preliminary evidence of activity in patients with immune-sensitive tumors.

Cohort Treated with 24 μg/Kg Dose

Ten individuals (male [100%], 6 [60.0%] Caucasian) with a median age of 61 years, ranging from 46-68 years of age, having advanced or metastatic solid tumors received the IL-2 conjugate at a 24 μg/kg dose Q3W. Tumor types included lung cancer, basal cell carcinoma, and colon cancer.

Each subject was treated with a) the IL-2 conjugate administered via IV infusion at a dose of 24 μg/kg for 30 minutes, and b) pembrolizumab administered at a dose of 200 mg IV sequentially. Treatment was given every 3 weeks [Q3W]. Effects on the same biomarkers described above for the 8 μg/kg and 16 μg/kg doses of the IL-2 conjugate were analyzed as surrogate predictors of safety and/or efficacy. Subjects in these studies met the same criteria as the subjects treated 8 μg/kg and 16 μg/kg doses.

All 10 subjects experienced at least one TEAE, and 7 (70.0%) of 10 subjects experienced at least 1 Grade 3-4 related TEAEs (1 Grade 3 and 6 Grade 4). There was one Grade 3 ALT/AST elevation (also with Grade 3 hypophosphatemia), G2 hyperbilirubinemia in the setting of G2 CRS, and 5 Grade 4 lymphocyte count decrease (one in a subject with Grade 3 AST/ALT elevation, Grade 2 hyperbilirubinemia-DLT along with Grade 2 CRS) and 1 G4 lymphopenia. The lymphocyte count recovered to at least Grade 3 in 48 hours.

Six subjects experienced related SAEs: one Grade 1 fever in a subject with adrenal insufficiency requiring steroid adjustment, and one Grade 2 cytokine release syndrome (fever and hypotension requiring fluids and dexamethasone) associated with Grade 3 AST/ALT elevation and G2 hyperbilirubinemia. Additionally, one subject developed G2 hypotension managed with supportive care, one subject developed an infusion related reaction during C1D1 followed by G2 CRS with fever, chills and hypotension managed with supportive care following cycle 2, an additional subject developed a G3 infusion related reaction during C1 followed by a cytokine release syndrome G1 during cycle 2, and one subject developed G2 CRS during cycle 2 managed with supportive care. There was one instance of a DLT: a subject with Grade 3 AST/ALT elevation along with Grade 2 hyperbilirubinemia associated with Grade 2 CRS (fever and hypotension requiring hydration and dexamethasone). For this subject, the dose was reduced for C2D1. No drug discontinuations resulted from TEAEs. TEAEs are detailed in Table 4.

TABLE 4
Treatment Emergent Adverse Events (TEAE) (n = 10)

| | 24 μg/kg Q3W + | |
|---|---|---|
| | pembrolizumab | |
| | (N = 10) | |
| Primary system organ class | All | Grade |
| Preferred Term n (%) | Grades | ≥3 |
| Number of Participants with TEAE | 10 (100) | 7 (70.0) |
| INFECTIONS AND INFESTATIONS | 1 (10.0) | 0 |
| BLOOD AND LYMPHATIC SYSTEM DISORDERS | 3 (30.0) | 1 (10.0) |
| IMMUNE SYSTEM DISORDERS | 3 (30.0) | 0 |
| ENDOCRINE DISORDERS | 0 | 0 |
| METABOLISM AND NUTRITION DISORDERS | 3 (30.0) | 1 (10.0) |
| PSYCHIATRIC DISORDERS | 1 (10.0) | 0 |
| NERVOUS SYSTEM DISORDERS | 1 (10.0) | 0 |
| EYE DISORDERS | 0 | 0 |
| CARDIAC DISORDERS | 0 | 0 |
| VASCULAR DISORDERS | 2 (20.0) | 0 |
| RESPIRATORY, THORACIC AND MEDIASTINAL | 0 | 0 |
| DISORDERS | | |
| GASTROINTESTINAL DISORDERS | 5 (50.0) | 0 |
| HEPATOBILIARY DISORDERS | 1 (10.0) | 0 |
| SKIN AND SUBCUTANEOUS TISSUE DISORDERS | 3 (30.0) | 0 |
| MUSCULOSKELETAL AND CONNECTIVE TISSUE | 2 (20.0) | 0 |
| DISORDERS | | |
| GENERAL DISORDERS AND ADMINISTRATION | 8 (80.0) | 1 (10.0) |
| SITE CONDITIONS | | |
| INVESTIGATIONS | 6 (60.0) | 6 (60.0) |
| INJURY, POISONING AND PROCEDURAL | 4 (40.0) | 1 (10.0) |
| COMPLICATIONS | | |

The following related events were reported: one Grade 3 AST/ALT and Grade 2 bilirubin (DLT) in the setting of Grade 2 CRS (fever, hypotension [BP97/56 mm Hg] and hypoxemia [SpO2 92%]) managed with fluid bolus, supplemental oxygen and dexamethasone with resolution required a dose reduction for C₂D1; one patient fever, chills, rigors and hypoxemia (92%) requiring supportive care and oxygen (C₂D1); one Grade 3 AST/ALT (C₂D8) presumed related to IL-2 conjugate and pembrolizumab without other symptoms in the setting of alcoholism; and three Grade 4 lymphocyte count decrease.

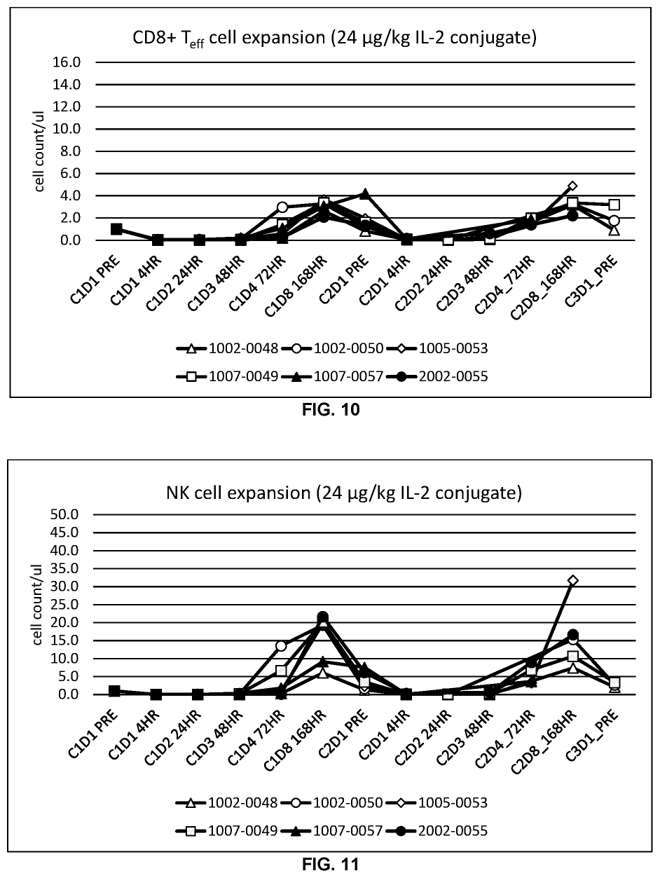

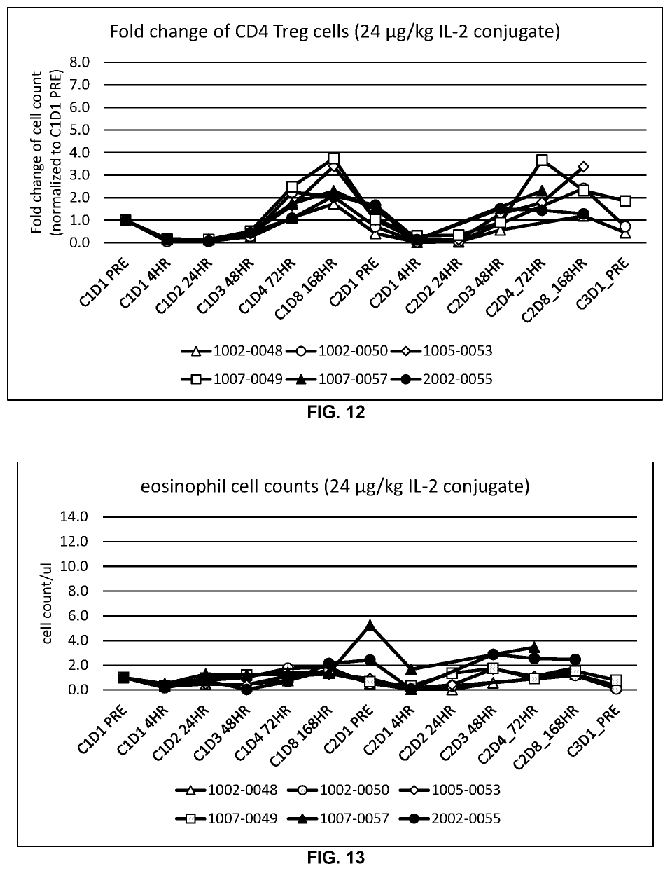

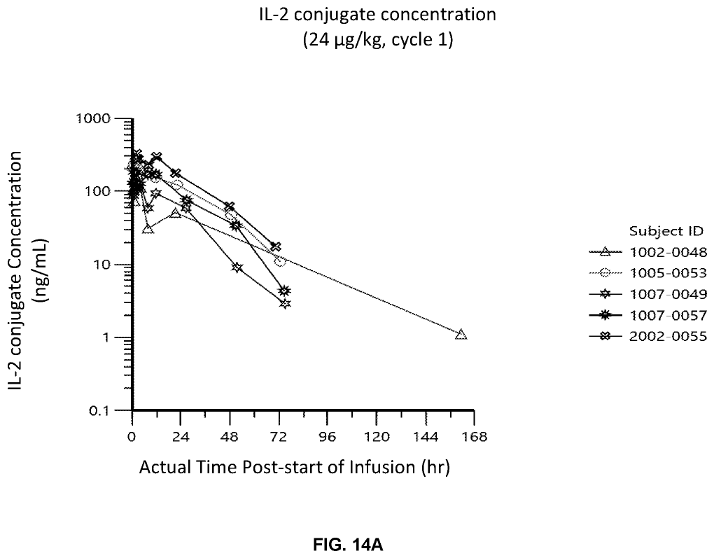

Efficacy biomarkers. Data relating to efficacy biomarkers was based on data available for 6 subjects receiving the IL-2 conjugate at 24 μg/kg. Peripheral CD8+T_{eff} cell counts were measured (FIG. 10), and peripheral NK cell counts are shown in FIG. 11. Peripheral CD4+ T_{reg} cell counts are shown in FIG. 12, and peripheral eosinophil cell counts are shown in FIG. 13.

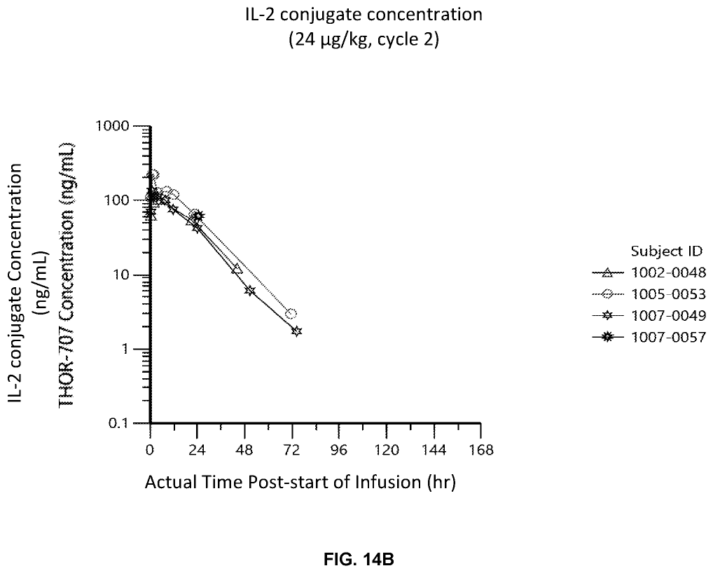

Mean concentrations of the IL-2 conjugate after 1 and 2 cycles are shown in FIG. 14A and FIG. 14B, respectively.

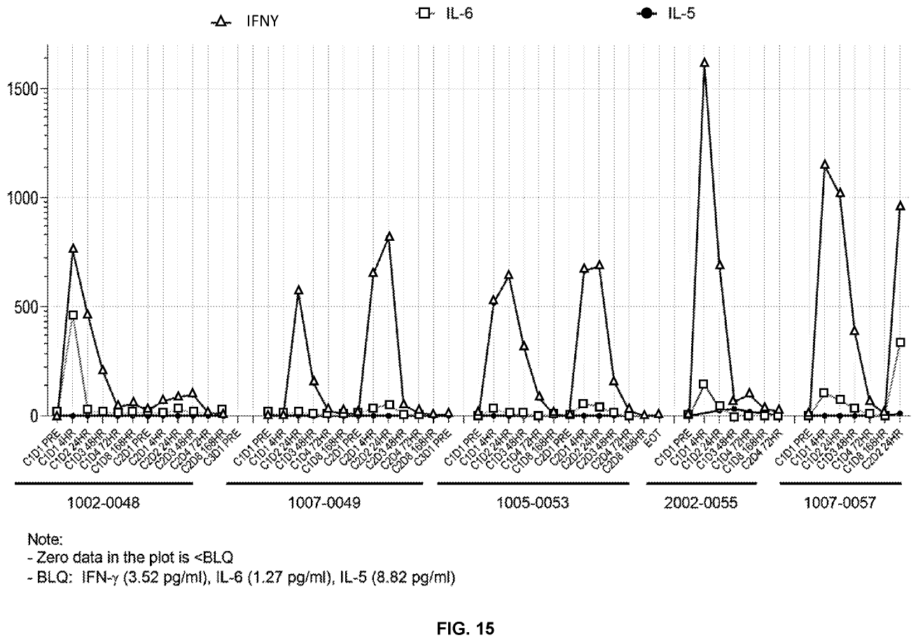

Cytokine levels (IFN-γ, IL-6, and IL-5) are shown in FIG. 15.

Accordingly, the IL-2 conjugate in combination with pembrolizumab demonstrated encouraging PD data and was generally well-tolerated with no discontinuations due to TRAE. Overall, the results are considered to support non-alpha preferential activity of the IL-2 conjugate, with a tolerable safety profile in combination with pembrolizumab as well as encouraging PD and preliminary evidence of activity in patients with immune-sensitive tumors.

Cohort Treated with 32 μg/Kg Dose

Three individuals having advanced or metastatic solid tumors received the IL-2 conjugate at a 32 μg/kg dose Q3W. Tumor types included ovarian carcinoma.

Each subject was treated with a) the IL-2 conjugate administered via IV infusion at a dose of 32 μg/kg for 30 minutes, and b) pembrolizumab administered at a dose of 200 mg IV sequentially. Treatment was given every 3 weeks [Q3W]. Effects on the same biomarkers described above for the 8 μg/kg and 16 μg/kg IL-2 conjugate doses were analyzed as surrogate predictors of safety and/or efficacy. Subjects in these studies met the same criteria as the subjects treated 8 μg/kg and 16 μg/kg doses.

All six (100%) subjects experienced at least one TEAE, and two (33.3%) of 6 subjects experienced at least 1 Grade 3-4 related TEAEs (1 Grade 4). There was one instance of Grade 4 lymphocyte count decrease (subject also had G3 fever). There were two related SAEs of Grade 1 fever and Grade 1 tachycardia requiring hospitalization for 24 hours (C₂D2-C₂D3), which resolved with supportive care. This patient had 3 additional SAEs of fever and tachycardia as well as an episode of G4 CRS on C₅ requiring hospitalization. This was resolved with supportive care. One additional patient developed G2 hypoxia during C1 requiring prolongation of hospitalization, which resolved with supportive care. There were no DLTs. One subject discontinued treatment as a result of the TEAE. TEAEs are detailed in Table 5.

TABLE 5
Treatment Emergent Adverse Events (TEAE) (n = 6)

| | 32 μg/kg IL-2 conjugate | |
|---|---|---|
| | Q3W + | |
| | pembrolizumab | |
| | (N = 6) | |
| Primary system organ class | All | Grade |
| Preferred Term n (%) | Grades | ≥3 |
| Number of Participants with TEAE | 6 (100) | 2 (33.3) |
| INFECTIONS AND INFESTATIONS | 0 | 0 |
| BLOOD AND LYMPHATIC SYSTEM | 0 | 0 |
| DISORDERS | | |
| IMMUNE SYSTEM DISORDERS | 2 (33.3) | 1 (16.7) |
| ENDOCRINE DISORDERS | 2 (33.3) | 0 |
| METABOLISM AND NUTRITION | 2 (33.3) | 0 |
| DISORDERS | | |
| PSYCHIATRIC DISORDERS | 0 | 0 |
| NERVOUS SYSTEM DISORDERS | 2 (33.3) | 0 |
| EYE DISORDERS | 1 (16.7) | 0 |
| CARDIAC DISORDERS | 1 (16.7) | 0 |
| VASCULAR DISORDERS | 1 (16.7) | 0 |
| RESPIRATORY, THORACIC AND | 2 (33.3) | 0 |
| MEDIASTINAL DISORDERS | | |
| GASTROINTESTINAL DISORDERS | 2 (33.3) | 0 |
| HEPATOBILIARY DISORDERS | 1 (16.7) | 0 |
| SKIN AND SUBCUTANEOUS TISSUE | 1 (16.7) | 0 |
| DISORDERS | | |
| MUSCULOSKELETAL AND CONNECTIVE | 0 | 0 |
| TISSUE DISORDERS | | |
| GENERAL DISORDERS AND | 5 (83.3) | 1 (16.7) |
| ADMINISTRATION SITE CONDITIONS | | |
| INVESTIGATIONS | 4 (66.7) | 1 (16.7) |
| INJURY, POISONING AND PROCEDURAL | 0 | 0 |
| COMPLICATIONS | | |

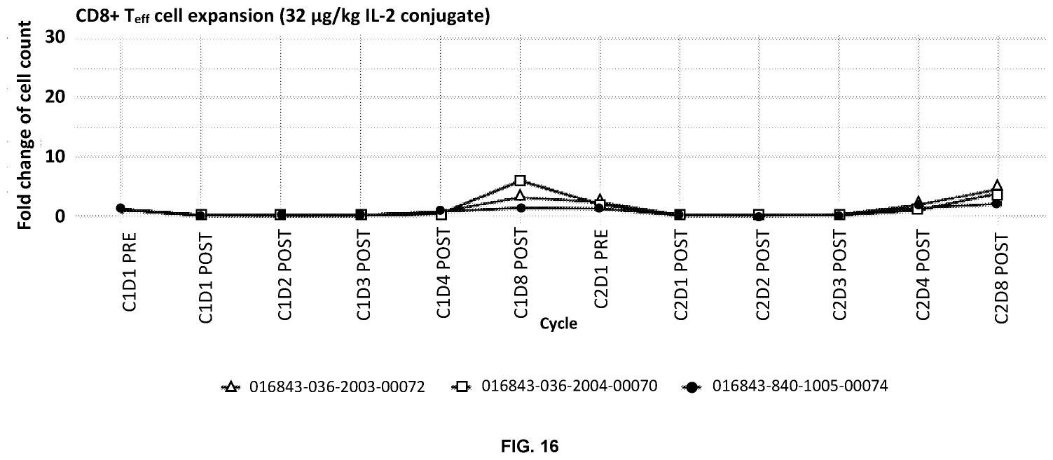

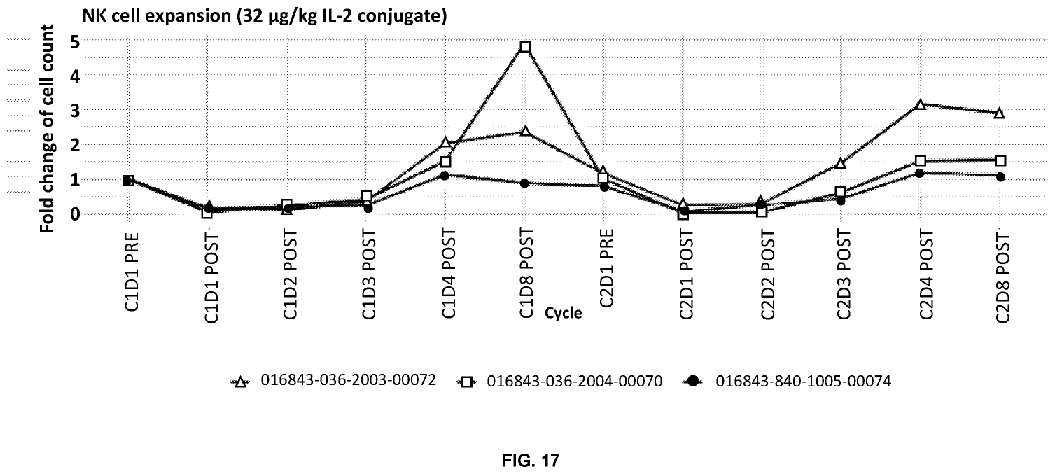

Efficacy biomarkers. Data relating to efficacy biomarkers was based on data available for 3 subjects receiving the IL-2 conjugate at 32 μg/kg. Peripheral CD8+T_{eff} cell counts were measured (FIG. 16). Peripheral CD4+ T_{reg} cell counts are shown in FIG. 17.

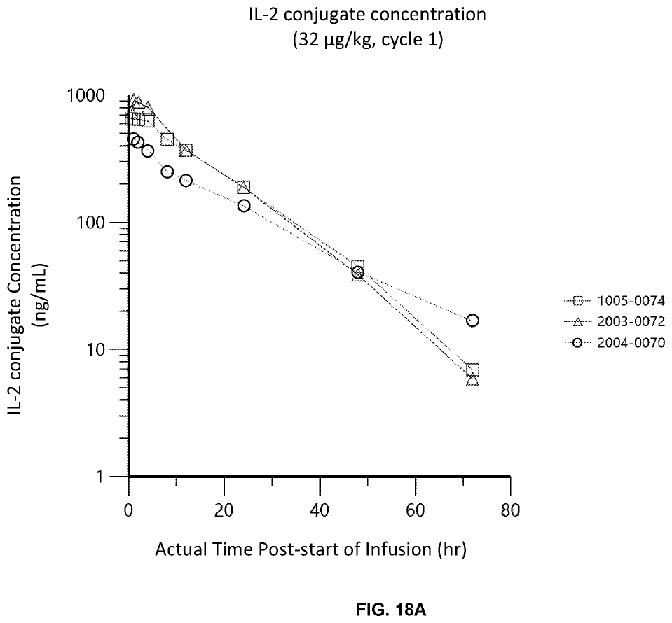

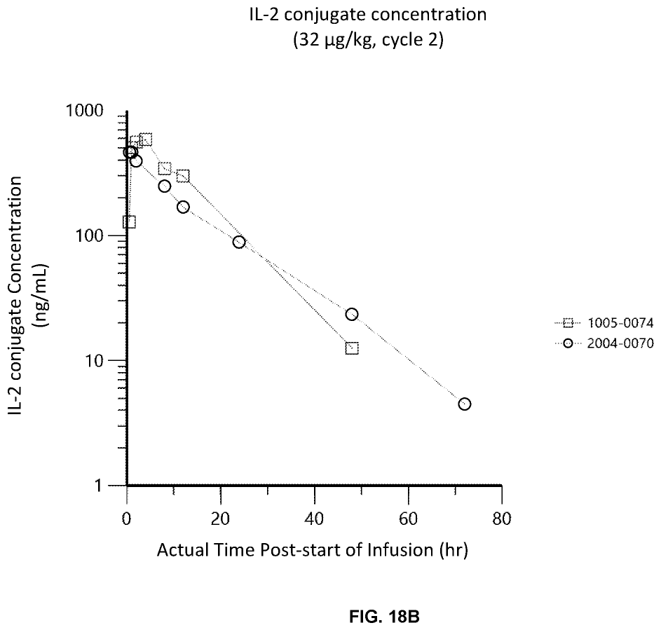

Mean concentrations of the IL-2 conjugate after 1 and 2 cycles are shown in FIG. 18A and FIG. 18B, respectively.

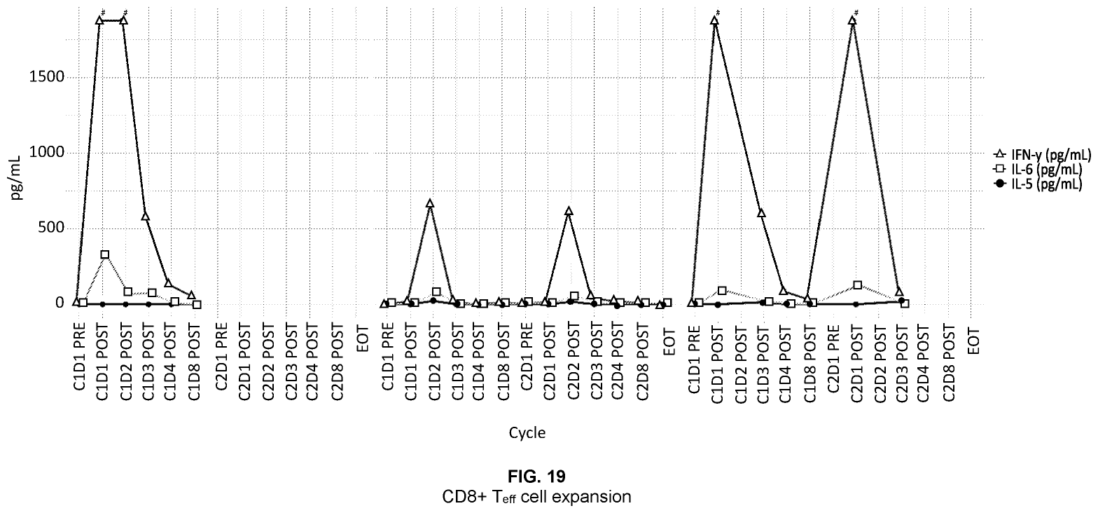

Cytokine levels (IFN-γ, IL-6, and IL-5) are shown in FIG. 19.

Accordingly, the IL-2 conjugate in combination with pembrolizumab demonstrated encouraging PD data and was generally well-tolerated with no discontinuations due to TEAE. Overall, the results are considered to support non-alpha preferential activity of the IL-2 conjugate, with a tolerable safety profile in combination with pembrolizumab as well as encouraging PD and preliminary evidence of activity in patients with immune-sensitive tumors.

### Administration of IL-2 Conjugate to a Subject Having Lung Cancer

### Q3W Dosing

One individual with NSCLC adenocarcinoma received the IL-2 conjugate (16 μg/kg) with pembrolizumab. Here and throughout Example 2, drug mass per kg subject (e.g., 16 μg/kg) refers to IL-2 mass exclusive of PEG and linker mass.

Peripheral CD8+T_{eff} cell counts, peripheral NK cell counts, peripheral CD4+ T_{reg} cell counts, lymphocyte cell counts, and eosinophil cell counts were measured in this subject before and after treatment as shown in Table 6. The subject showed stable disease at target lesions after two treatment cycles, and continued stable disease after five cycles. Non-target lesions showed a complete response.

TABLE 6
Normalized Peripheral CD8+ T_{eff} cell counts, peripheral NK cell counts, peripheral
CD4+ T_{reg} cell counts, lymphocyte cell counts, and eosinophil cell counts.

| | Cycle 1 of treatment | | | | | Cycle 2 of treatment | | | |
|---|---|---|---|---|---|---|---|---|---|
| | time after treatment ^{a} | | | | | time after treatment ^{a} | | | |
| Cell type | 0 hr | 4 hr | 24 hr | 48 hr | 168 hr | 0 hr | 4 hr | 24 hr | 168 hr |
| Peripheral | 1.00 | 0.02 | 0.02 | 0.12 | 3.29 | 0.57 | 0.03 | 0.02 | 1.70 |
| CD8+ T_{eff} cells | | | | | | | | | |
| Peripheral NK | 1.00 | 0.01 | 0.01 | 0.16 | 26.85 | 2.23 | 0.03 | 0.06 | 13.99 |
| cells | | | | | | | | | |
| Peripheral | 1.00 | 0.06 | 0.03 | 0.22 | 3.19 | 0.54 | 0.03 | 0.04 | 1.88 |
| CD4+ T_{reg} cells | | | | | | | | | |
| Lymphocytes | 1.00 | 0.05 | 0.03 | 0.22 | 6.36 | 0.78 | 0.04 | 0.04 | 3.45 |
| Eosinophils | 1.00 | 0.17 | 0.42 | 1.62 | 0.98 | 0.44 | 0.07 | 0.18 | 1.66 |

^{a} 0 hr refers to cell count determination just prior to cycle treatment.

IFN-γ, IL-6, and IL-5 levels were measured in this subject before and after treatment as shown in FIG. 8D (subject 1001-0026). The measured values indicate that IFN-γ was induced, but low amounts of IL-5 and IL-6, cytokines associated with VLS and CRS, respectively, were induced.

### Example 3. Use of an IL-2 Conjugate Plus a Checkpoint Inhibitor in the Treatment of CT-26 Tumor-Bearing Balb/c Mice

An IL-2 conjugate “IL-2 P65[AzK_PEG30kD]” (also referred to herein and in the Figures as “Compound A”) comprising SEQ ID NO: 3 was used in this study:

(SEQ ID NO: 3)
APTSSSTKKTQLQLEHLLLDLQMILNGINNYKNPKLTRMLTFKFYMPKK
ATELKHLQCLEEELK[AzK PEG30kD]LEEVLNLAQSKNFHLRPRDLI
SNINVIVLELKGSETTFMCEYADETATIVEFLNRWITFSQSIISTLT

wherein [AzK_PEG30kD] is N6-((2-azidoethoxy)-carbonyl)-_{L}-lysine stably-conjugated to PEG via DBCO-mediated click chemistry to form compounds comprising a structure of Formula (I), supra, in which Z is CH₂, Y is

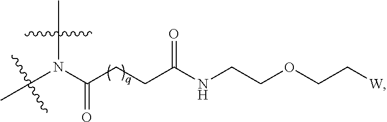

q is 3, and W is a methoxy, linear PEG group having an average molecular weight of 30 kDa and/or compounds comprising a structure of Formula (I) wherein Y is CH₂ and Z is

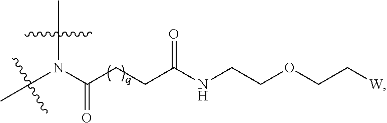

q is 3, and W is a methoxy, linear PEG group having an average molecular weight of 30 kDa. The compound was prepared using methods wherein a protein was first prepared having SEQ ID NO: 4 in which the proline at position 65 was replaced by N6-((2-azidoethoxy)-carbonyl)-_{L}-lysine AzK.

(SEQ ID NO: 4)
APTSSSTKKTQLQLEHLLLDLQMILNGINNYKNPKLTRMLTFKFYMPKK
ATELKHLQCLEEELKPLEEVLNLAQSKNFHLRPRDLISNINVIVLELKG
SETTFMCEYADETATIVEFLNRWITFSQSIISTLT

The AzK-containing protein was then allowed to react under click chemistry conditions with DBCO comprising a methoxy, linear PEG group having an average molecular weight of 30 kDa, followed by purification and formulation employing standard procedures.

Studies of Compound A as monotherapy and in combination with an anti-PD-1 antibody were undertaken in Balb/c female mice. Balb/c female mice, 6-8 weeks of age, with an average weight of 16 g to 21 g were purchased from Jackson Laboratories (Sacramento, CA) for studies 1 and 2. Balb/c female mice, 7-8 weeks of age, with an average weight of 18 to 22 g were purchased from Taconic Biosciences by HD Biosciences for study 3. Cryogenically preserved vials of CT-26 colon cancer cells were purchased from American Tissue Type Collection (ATCC, Manassas, VA). Cells were thawed and cultured according to the manufacturer's protocol. On the day of tumor cell inoculation, cells were washed in serum-free media, counted, and resuspended in cold serum-free media at a concentration of 250,000 (studies 1 and 2) or 300,000 (study 3) viable cells per 0.1 mL. The CT-26 cells (0.1 mL) were injected subcutaneously into the flanks of individual mice and tumors were allowed to grow.

For studies 1 and 2 in which a combination of Compound A and an anti-PD-1 antibody were used, the antibody used was anti-mouse PD-1 (BioXcell; RMP1-14) and the control antibody was IgG1 isotype antibody (BioXcell; catalog #BP0089, lot #2A3). For study 3 in which an anti-PD-1 antibody was used, the antibody used was anti-mouse PD-1 (BioXcell; catalog #BP0146, RMP1-14, lot #695318A1) and the control antibody was IgG1 isotype antibody (BioXcell; catalog #BP0089, lot #2A3).

Lyophilized Compound A was reconstituted into 10 mg/mL stock with 0.1 M acetic acid. It was then further diluted into working concentration with 1x phosphate buffered saline (PBS). The compound was reconstituted and diluted within an hour of dosing of animals and kept on ice until dosing. The lyophilized compound was stored at −80C before use. Vehicle was stored at 4VC.

Three separate efficacy studies were performed using CT-26 tumor-bearing Balb/c mice. The design for study 1, which evaluated Compound A for dose-dependent efficacy as a single agent, is outlined in Table 7. The designs for studies 2 and 3, which evaluated the efficacy of Compound A in combination with an anti-PD-antibody, are outlined in Table 8 and Table 9, respectively. The route of administration for Compound A was intravenous (IV). IV dosing in the mice was done via the tail vein. The antibody was administered intraperitoneally (IP). All agents were administered based on the individual body weight of each animal obtained immediately prior to each dosing. Details on the dosing regimen are described below.

TABLE 7
Study #1: Control and Test Treatment Groups in CT-26
Tumor-Bearing Mice.

| Agent | Dose (mg/kg) | Route, Schedule | No. of mice |
|---|---|---|---|
| Vehicle control | 0 | IV, QWx3 | 10 |
| Compound A | 0.3 | IV, QWx3 | 10 |
| Compound A | 0.3 | IV, Q2Wx2 | 10 |
| Compound A | 1 | IV, QWx3 | 10 |
| Compound A | 1 | IV, Q2Wx2 | 10 |
| Compound A | 3 | IV, QWx3 | 10 |
| Compound A | 3 | IV, Q2Wx2 | 10 |

IV = intravenous;
QWx3 = once a week for a total of 3 doses;
Q2Wx2 = once every 2 weeks for a total of 2 doses.

TABLE 8
Study #2: Control and Test Treatment Groups in CT-26
Tumor-Bearing Mice.

| Agent | Dose (mg/kg) | Route, Schedule | No. of mice |
|---|---|---|---|
| Vehicle control + | 0 + 10 | IV, QWx3 + IP, | 14 |
| IgG isotype control | | BIWx3 | |
| Compound A | 3 | IV, QWx3 | 14 |
| Compound A | 6 | IV, QWx3 | 14 |
| Anti-PD-1 antibody | 10 | IP, BIWx3 | 14 |
| Compound A + | 6 + 10 | IV, QWx3 + IP, | 14 |
| Anti-PD-1 antibody | | BIWx3 | |

BIWx3 = twice a week for 3 weeks with a total of 6 doses;
IP = intraperitoneal;
IV = intravenous;
QWx3 = once a week for a total of 3 doses.

TABLE 9
Study #3: Control and Test Treatment Groups in CT-26
Tumor-Bearing Mice.

| Agent | Dose (mg/kg) | Route, Schedule | No. of mice |
|---|---|---|---|
| Vehicle control + IgG | 0 + 10 | IV, QWx3 + IP, | 14 |
| isotype control | | BIWx3 | |
| Compound A | 1 | IV, QWx3 | 14 |
| Compound A | 3 | IV, QWx3 | 14 |
| Compound A | 6 | IV, QWx3 | 14 |
| Compound A | 9 | IV, QWx3 | 14 |
| Anti-PD-1 antibody | 10 | IP, BIWx3 | 14 |
| Compound A + Anti-PD-1 | 1 + 10 | IV, QWx3 + IP, | 14 |
| antibody | | BIWx3 | |
| Compound A + Anti-PD-1 | 3 + 10 | IV, QWx3 + IP, | 14 |
| antibody | | BIWx3 | |
| Compound A + Anti-PD-1 | 6 + 10 | IV, QWx3 + IP, | 14 |
| antibody | | BIWx3 | |

BIWx3 = twice a week for 3 weeks with a total of 6 doses;
IP = intraperitoneal;
IV = intravenous;
QWx3 = once a week for a total of 3 doses;
Q2Wx2 = once every 2 weeks for a total of 2 doses.

In Study 1, CT-26 tumor-bearing mice were treated with vehicle IV once a week for a total of 3 doses (QWx3) or Compound A at 0.3, 1, or 3 mg/kg IV, either once a week for a total of three doses (QWx3), or once every 2 weeks for a total of 2 doses (Q2Wx2), starting on Day 4 following tumor cell inoculation when the average tumor volume was ˜80 mm³.

In Study 2, CT-26 tumor-bearing mice were treated on Day 5 following tumor cell inoculation when the average tumor volume was ˜80 mm³. Dosing was with vehicle IV QWx3+IgG isotype control IP or Compound A at 3 or 6 mg/kg IV, on a QWx3 dosing schedule, or anti-PD-1 antibody at 10 mg/kg IP, or the combination of Compound A at 6 mg/kg IV QWx3+anti-PD-1 antibody at 10 mg/kg IP. The IP dosing of the antibody in all cases was twice a week for 3 weeks with a total of 6 doses (BIWx3).

In Study 3, CT-26 tumor-bearing mice were treated on Day 7 following tumor cell inoculation when the average tumor volume was ˜70 mm3. Dosing was with vehicle IV QWx3+IgG isotype control IP BIWx3; or Compound A at 1, 3, 6, or 9 mg/kg IV on a QWx3 dosing schedule, or anti-PD-1 antibody at 10 mg/kg IP BIWx3; or the combination of Compound A at 1, 3, or 6 mg/kg IV QWx3+anti-PD-1 antibody at 10 mg/kg IP BIWx3.

A summary of all three studies is shown in Table 10. Animals were observed daily for clinical signs. In accordance with IACUC guidelines, animals were humanely euthanized when tumors grew over 2000 mm³ in volume or they were observed to have a continuing deteriorating condition or showing obvious signs of severe distress and/or pain.

The survival of each mouse was monitored for over 100 days, at which time surviving tumor-free animals in Studies 2 and 3 were included in a re-challenge continuation of the study for two cycles, 2 months apart. Specifically, tumor-free animals were re-challenged via inoculation of the same type of tumor cells (CT-26) in the opposite lower flank. Control animals were age-matched naïve mice that were concurrently inoculated with the same number of CT-26 tumor cells in the opposite lower flank.

Tumor growth was monitored using digital caliper measurements every 3 to 4 days until the end of the study. Tumor volume was calculated as Width²× Length/2, where width is the smallest dimension and length is the largest. Raw tumor volume data are presented in the study reports.

Mean tumor volume data for each group was plotted over time with standard error of the mean (SEM) bars. Additionally, individual tumor volume data for the last day before animal sacrifice was plotted along with mean and SEM bars to examine the distribution of the data.

A statistical analysis of the tumor volume data for the last day before animal sacrifice was performed using the using GraphPad Prism v.7.0. Data was analyzed for significance using a one-way ANOVA. Pairwise comparisons were made using Tukey's test procedures (2-sided). The p-value for each individual comparison was reported.

The percent tumor growth inhibition (% TGI) in each treated group vs. a control group was calculated as:

[(Control-Control baseline)−(Treated-Treated baseline)]/(Control-Control baseline)×100%.

The survival of each mouse was recorded, and a Kaplan-Meir plot was generated to show survival by treatments group and the significance was assessed by log-rank (Mantel-Cox) test. Survival was monitored for over 100 days following treatment initiation in Studies #1, #2, and #3, and over the two re-challenge cycles in surviving tumor-free mice in Studies #2 and #3. Analyses were performed using GraphPad Prism version 7.0.

TABLE 10
Tumor Growth Inhibition in Mice with Compound A as Monotherapy
and in Combination with Anti-Mouse PD-1 Antibody.

| | Compound A | % TGI (Relative to Vehicle Control) | | | |
|---|---|---|---|---|---|
| | Dose [Antibody | Study #1 | | Study #2 | Study #3 |
| | Doseª] (mg/kg) | QWx3 | Q2Wx2 | QWx3 | QWx3 |
| Compound A | 0.3 | 19 | 20 | — | — |
| Monotherapy | 1 | 31 | 27 | — | 30 |
| | 3 | 51* | 45* | 56 | 59*** |
| | 6 | — | — | 36* | 86*** |
| | 9 | — | — | — | 85*** |
| | Antibody [10] | — | — | 44^{#} | 44^{#} |
| Combination | 6 + [10] | — | — | 75** | 84* |
| Therapy^{b} | | | | | |

ªDosed BIW for 3 weeks (6 total doses);
^{b}Data for the 1 and 3 mg/kg Compound A combination groups not shown.
% TGI was calculated on Day 15 (Study 1) and Day 17 (Studies 2 and 3).
Results are mean ± SEM.
QWx3 = once a week for a total of 3 doses;
Q2Wx2 = once every 2 weeks for a total of 2 doses;
TGI = tumor growth inhibition.
*p < 0.05 vs. vehicle control;
**p < 0.05 vs. monotherapies (Compound A or antibody);
***p < 0.001 vs. vehicle or antibody isotype control;
^{#}p < 0.01 vs. antibody isotype control.

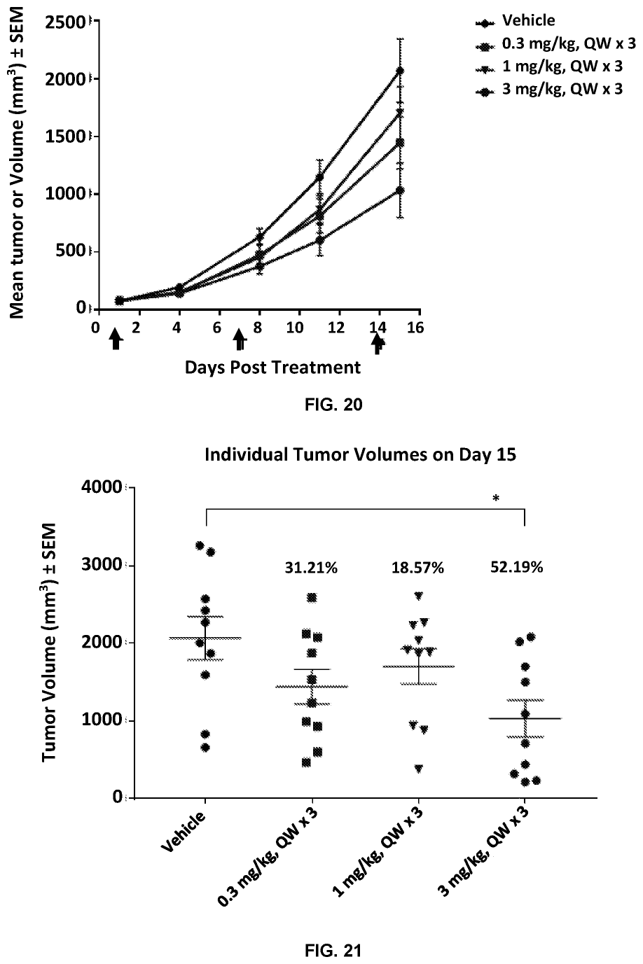

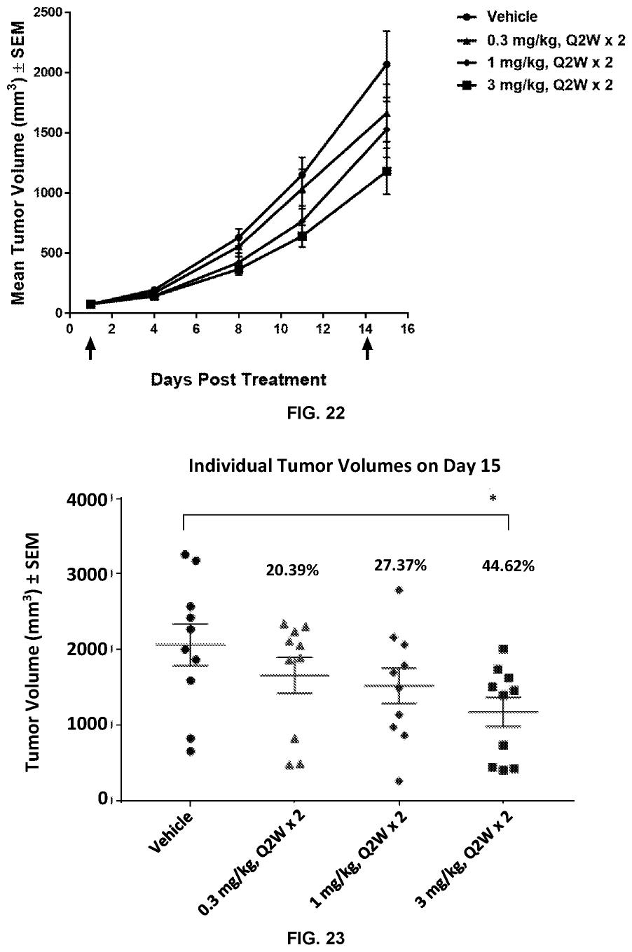

In Study 1, Compound A was evaluated for dose-dependent efficacy as a single agent in female Balb/c mice bearing subcutaneously established CT-26 colon tumors. The study formally ended on Day 15 after treatment initiation according to the humane endpoint set forth by the IACUC when several tumors in the control group reached over 2000 mm3 in volume. FIG. 20 shows mean tumor volume over time for groups treated QWx3 dosing with Compound A. FIG. 21 shows tumor volumes on Day 15 post treatment for each animal treated QWx3 dosing with Compound A. FIG. 22 shows mean tumor volume over time for groups treated Q2Wx2 dosing with Compound A. FIG. 23 shows tumor volumes on Day 15 post treatment for each animal with Q2Wx2 dosing with Compound A.

On the QWx3 dosing schedule, Compound A demonstrated dose-dependent single agent anti-tumor activity resulting in % TGI compared to the vehicle control of 31%, 19%, and 52% for the 0.3, 1, and 3 mg/kg dose groups, respectively. Similarly, on the Q2Wx2 dosing schedule, Compound A demonstrated dose-dependent single agent anti-tumor activity resulting % TGI compared to the vehicle control of 20%, 27%, and 45% for the 0.3, 1, and 3 mg/kg dose groups, respectively. However, on both dosing schedules, only the 3 mg/kg dose was statistically significant (p<0.05) compared to the vehicle control. Both dosing schedules demonstrated comparable anti-tumor activity. Therefore, for the subsequent studies in this mouse model, the QWx3 dosing schedule was chosen.

In FIGS. 20, 22, 24, and 27, black arrows denote days of Compound A dosing. Data in FIGS. 20 and 22 are mean tumor growth curves with QWx3 dosing and Q2Wx2 dosing with Compound A; black arrows denote days of Compound A dosing. Data in FIGS. 21 and 23 represent individual tumor volume and mean tumor volume±standard error of the mean (SEM) (10 mice/group) on day 15 post-treatment with QWx3 and Q2Wx2 dosing with Compound A. Data represent individual tumor volumes; the mean±SEM and % TGI compared to the vehicle control are also displayed.

Data in FIG. 22 represents mean tumor volume±standard error of the mean (SEM) (10 mice/group) in animals with Q2Wx2 dosing with Compound A. Data in FIG. 23 represents individual and mean tumor volume data on Day 15 post treatment with Q2Wx2 dosing with Compound A. * p<0.05 vs. vehicle control on Day 15.

There were two separate studies (Studies 2 and 3) conducted in CT-26 colon tumor-bearing mice to assess Compound A as a single agent and in combination with a murine anti-PD-1 checkpoint inhibitor antibody. The dose ranges for Compound A between the studies overlapped, with Study 3 having a wider dose range. In both studies, Compound A was administered QWx3 and the same dose level of antibody was administered BIWx3.

In Study 2, anti-tumor activity of Compound A was evaluated as a single agent at 3 and 6 mg/kg (QWx3) in female Balb/c mice bearing subcutaneously established CT-26 colon tumors. Additionally, the combination anti-tumor activity was evaluated with IV dosing of Compound A at 6 mg/kg (QWx3) and anti-PD-1 antibody at 10 mg/kg IP (BIWx3). The % TGI was calculated on Day 15 after treatment initiation because several tumors in the vehicle control group reached over 2000 mm³ in volume. However, the animals in treatment groups that demonstrated complete tumor regression were followed with tumor measurements at a frequency of once or twice a week.

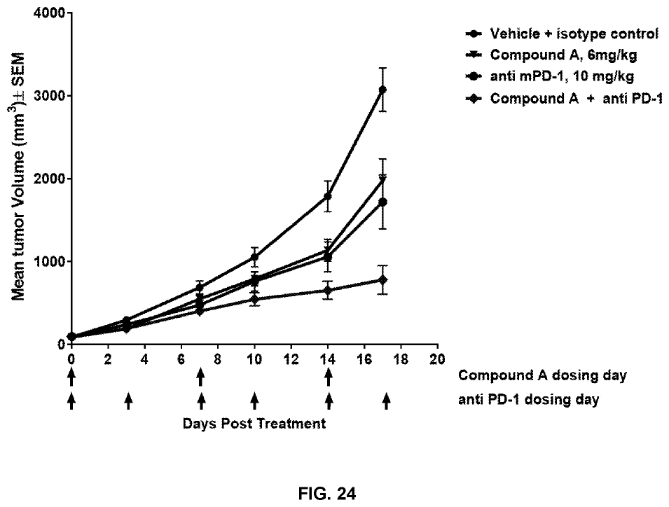

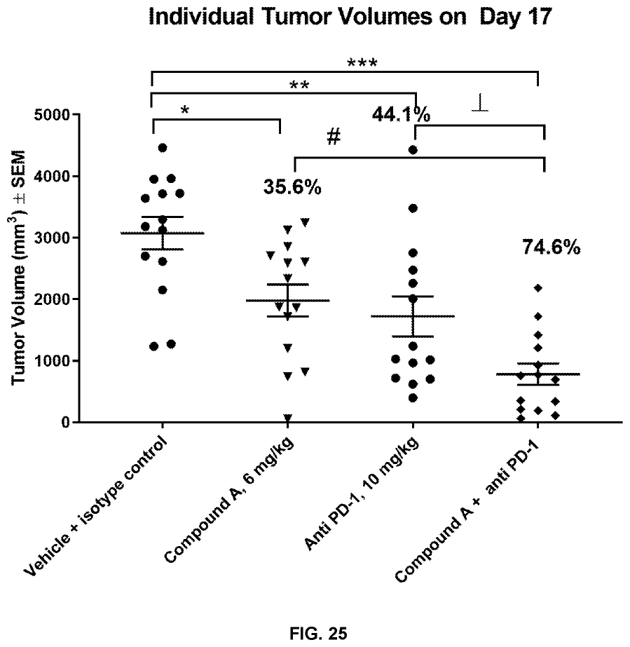

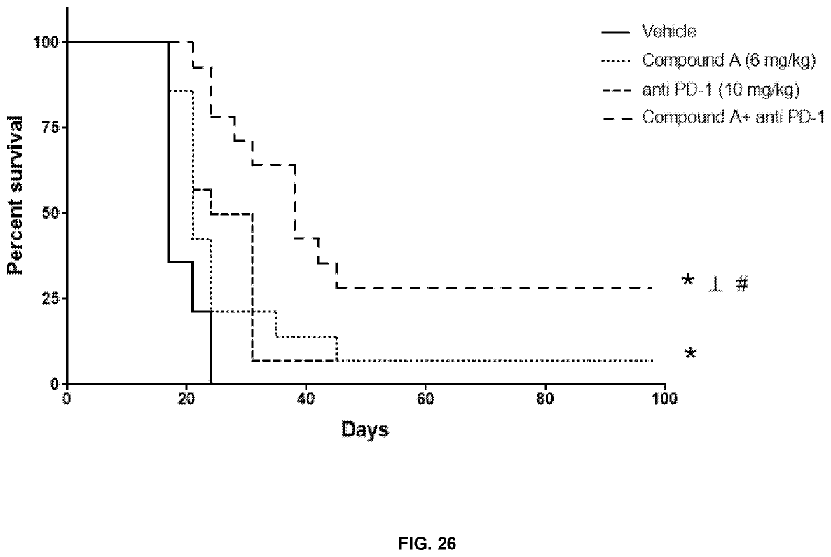

Compound A demonstrated single agent anti-tumor activity resulting in % TGI compared to the vehicle control of 56.3% and 35.6% for the 3 and 6 mg/kg dose groups, respectively. In the combination study, CT-26 tumor-bearing mice were treated IV with Compound A at 6 mg/kg QWx3, or IP with anti-PD-1 antibody BIWx3, or the combination with the same dosing schedules, starting 5 days following tumor cell inoculation when the average tumor volume was ˜80 mm³. Mean tumor growth curves are shown in FIG. 24 for treatment of mice with vehicle, 6 mg/kg Compound A as a single agent, anti-PD-1 antibody as a single agent, and the combination of 6 mg/kg Compound A and anti-PD-1 antibody. Data in FIG. 24 represent mean tumor volume±SEM (14 mice/group). Upper arrows denote days of Compound A dosing and lower arrows denote days of anti-PD-1 antibody dosing. The combination anti-tumor activity was significantly enhanced compared to Compound A or anti-PD-1 antibody alone (p<0.05). The % TGI data is shown in FIG. 25 and shows significant anti-tumor effects on Day 15 post treatment in the group treated with the combination of Compound A and anti-PD-1 antibody, compared to the groups treated with vehicle, Compound A alone or the anti-PD-1 antibody alone (35.6% for the Compound A alone group; 44.1% for the anti-PD-1 antibody alone group; and 74.6% for the group administered the combination of Compound A and anti-PD-1 antibody). Data represent individual tumor volumes; the mean±SEM and % TGI compared to the vehicle control are also displayed. *p<0.05, **p<0.01, and ***p<0.01; vs. vehicle control. ^{⊥}p<0.05 vs. anti-PD-1 antibody. #p<0.05 vs. Compound A. The median survival times of the groups are shown in FIG. 26 and were 17, 27, 27.5, and 38 days for the control, Compound A, anti-PD-1 antibody, and Compound A+anti-PD-1 antibody groups, respectively. The median survival time of the combination group was significantly longer than both the Compound A (p<0.05) and anti-PD-1 antibody (p<0.05) single agent treatment groups. At 98 days post treatment, only 1 out of 14 animals (7%) in each of Compound A and anti-PD-1 antibody dose groups survived tumor-free, while 4 of 14 animals (29%) in the combination group survived tumor-free. Data in FIG. 26 represent Kaplan-Meier survival curves for treatment groups. *p<0.05 vs. vehicle control. ^{⊥}p<0.05 vs. anti-PD-1 antibody. #p<0.05 vs. Compound A.

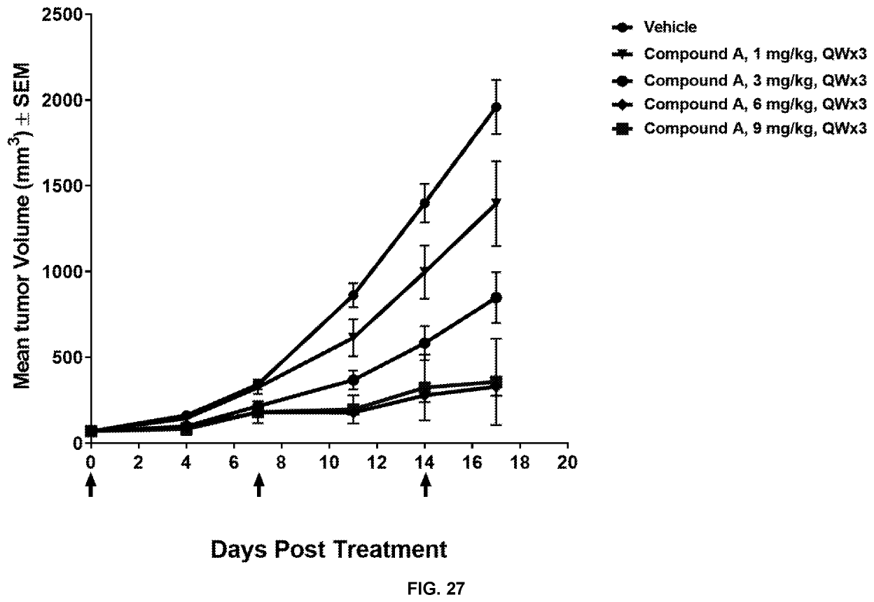

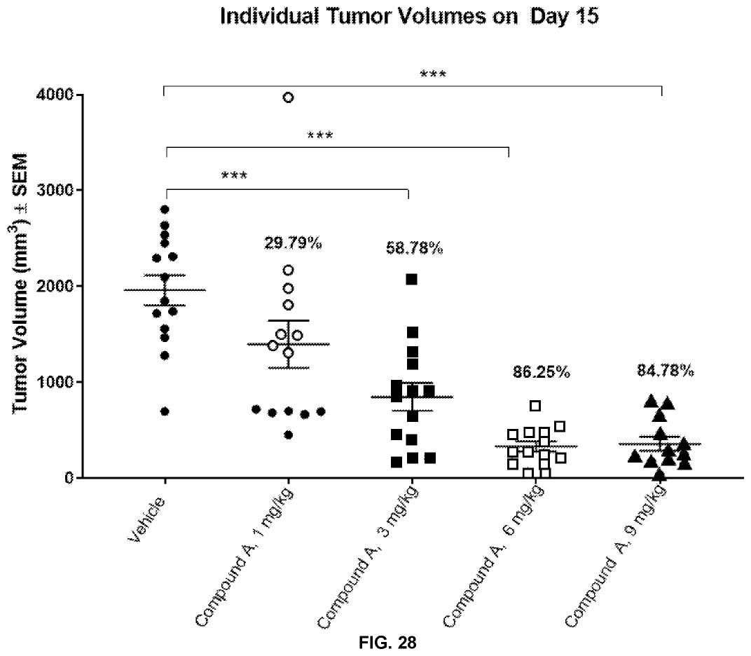

In Study 3, the single agent anti-tumor activity of Compound A was evaluated in female Balb/c mice bearing SC CT-26 colon tumors at a wider dose range (1, 3, 6, and 9 mg/kg) as compared to Study 2 on the same IV QWx3 dosing schedule. Data in FIG. 27 represent mean tumor growth curves when Compound A was dosed a single agent at 1 mg/kg, 3 mg/kg, 6 mg/kg, and 9 mg/kg. Data represent mean tumor volume±SEM (14 mice/group; except 12 mice/group for 9 mg/kg Compound A). Black arrows denote days of Compound A dosing. Compound A dosed alone at 1 mg/kg, 3 mg/kg, 6 mg/kg, and 9 mg/kg also demonstrated dose-dependent anti-tumor activity resulting in % TGI compared to the vehicle control of 29.8%, 58.8%, 86.2%, and 84.8% for 1, 3, 6, and 9 mg/kg dose groups, respectively (FIG. 28). The % TGI was calculated on Day 15 after treatment initiation because several tumors in the vehicle control group reached over 2000 mm³. However, the animals in treatment groups that demonstrated complete tumor regression were followed with tumor measurements at a frequency of once or twice a week. Data in FIG. 28 represent individual tumor volumes on Day 15 post treatment. Data represent individual tumor volumes; the mean±SEM and % TGI compared to the vehicle control are also displayed. ***p<0.01 vs. vehicle control. The lowest dose (1 mg/kg) did not show statistically significant anti-tumor activity while the other 3 dose groups were statistically significant (p<0.001) compared to the vehicle treated group. The data also showed that % TGI for the two high dose groups (6 mg/kg and 9 mg/kg) were similar indicating maximal anti-tumor activity was reached at the 6 mg/kg dose. In the 9 mg/kg dose group, 2 of the 14 animals were found dead following >15% body weight loss due to treatment.

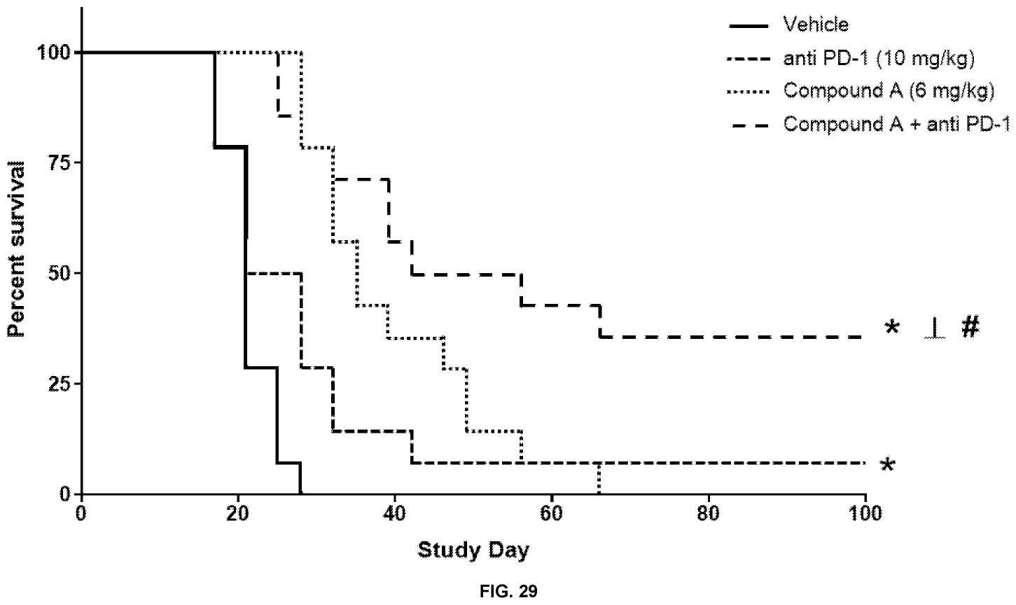

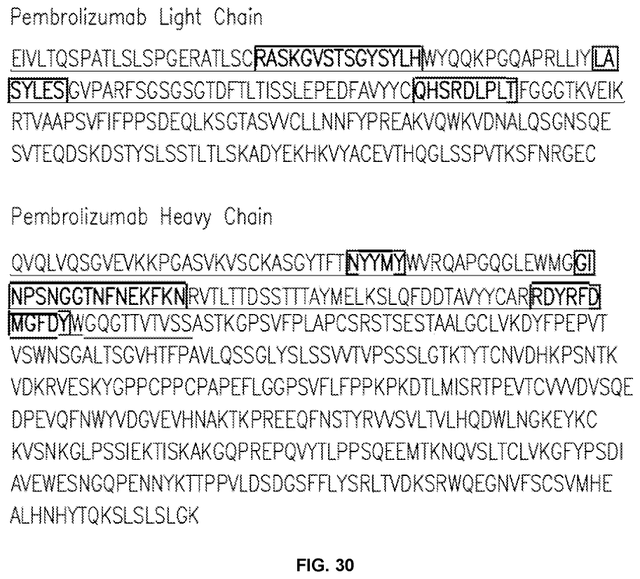

In the combination phase of the study, Compound A at 1, 3, or 6 mg/kg (QWx3) was dosed with anti-PD-1 antibody at 10 mg/kg IP (BIWx3). CT-26 tumor-bearing mice were treated IV with Compound A at 1, 3, 6, or 9 mg/kg QWx3, or IP anti-PD-1 antibody BIWx3, or the combination with the same dosing schedules, starting 7 days following tumor cell inoculation when the average tumor volume was ˜70 mm3. Note that for the 9 mg/kg Compound A single agent group, two animals were found dead after >15% body weight loss and are not included in the analysis. With the combination of 1 mg/kg Compound A+anti-PD-1 antibody, no additive anti-tumor activity was observed based on survival data. At Compound A days post treatment, 1 of 14 animals (7%) in the anti-PD-1 antibody group survived, while 0 of the animals in the 3 mg/kg single agent group survived. However, in the 3 mg/kg Compound A +anti-PD-1 antibody group, 2 of 14 animals (14%) survived up to Compound A days. As shown in FIG. 29, the combination of 6 mg/kg Compound A+anti-PD-1 antibody resulted in prolonged survival compared to each single agent alone. The median survival times were 21, 35, 24.5, and 49 days for the vehicle control, Compound A (6 mg/kg), anti-PD-1 antibody (10 mg/kg), and Compound A+anti-PD-1 antibody groups (6 mg/kg Compound A and 10 mg/kg anti-PD-1 antibody), respectively. The median survival time of the combination group was significantly longer than the Compound A and anti-PD-1 antibody (p<0.05) single agent treatment groups. Specifically, at Compound A days post treatment, 0 of the animals in the 6 mg/kg Compound A group survived while only 1 of 14 animals (7%) in the anti-PD-1 antibody group survived tumor-free. However, in the combination group, 5 of 14 (36%) animals survived tumor-free (p<0.05). Data in FIG. 29 represent Kaplan-Meier survival curves for treatment groups. *p<0.05 vs. vehicle control. ^{⊥}p<0.05 vs. anti-PD-1 antibody. #p<0.05 vs. Compound A.

### Example 4. Clinical Study of Combination Therapy Using an IL-2 Conjugate and Pembrolizumab

A Phase 2 non-randomized, open-label, multi-cohort, multi-center study assessing the clinical benefit of the IL-2 conjugate described in Example 2 in combination with pembrolizumab for the treatment of participants with advanced lung cancer or pleural mesothelioma is undertaken. Cohort A1 participants are patients with stage IV NSCLC having a PD-L1 tumor proportion score (TPS) greater than or equal to 50% who have not received prior treatment (i.e., the IL-2 conjugate treatment is 1L or first-line therapy; the subject is treatment-naïve). Cohort A2 participants are patients with stage IV NSCLC having a PD-L1 tumor proportion score (TPS) of 1-49% who have not received prior treatment (i.e., the IL-2 conjugate treatment is 1L or first-line therapy; the subject is treatment-naïve). Cohort A3 participants are patients with stage IV non-squamous NSCLC who have not received prior treatment (i.e., the IL-2 conjugate treatment is 1L or first-line therapy; the subject is treatment-naïve). Cohort B1 participants are patients with stage IV NSCLC who have received one or two prior lines of therapy (i.e., the IL-2 conjugate treatment is 2/3L, or second- or third-line therapy) and who have progressed on a checkpoint inhibitor (CPI)-based therapy, such as PD-1/PD-L1, and for whom based on investigator judgment, either docetaxel or pemetrexed is not the best treatment. Cohort B2 participants are patients with stage IV NSCLC who have received one or two prior lines of therapy (i.e., the IL-2 conjugate treatment is 2/3L, or second- or third-line therapy) and who have progressed on a checkpoint inhibitor (CPI)-based therapy, such as PD-1/PD-L1, and for whom based on investigator judgment, either docetaxel or pemetrexed is not the best treatment. Cohort C1 participants are patients with unresectable malignant pleural mesothelioma who have received one or two prior lines of therapy that include pemetrexed-based regimen in combination with a platinum agent (i.e., the IL-2 conjugate treatment is 2/3L, or second- or third-line therapy) and are CPI naïve.

Cohort B1 and B2 participants previously received one anti-PD-1/PD-L1 containing regimen, which may have included chemotherapy agents as part of the regimen, to treat stage IV NSCLC which progressed, after documented benefit, on an anti-PD-1/PD-L1 containing regimen per RECIST 1.1. The documentation of benefit from an anti-PD-1/PD-L1 containing regimen is defined as SD at >1 radiographic imaging scan, CR, or partial response (PR). An anti-PD-1/PD-L1 containing regimen is defined as either an anti-PD-1/PD-L1 monotherapy or an anti-PD-1/PD-L1 agent administered in the same cycle as another systemic anticancer therapy. If PD-1/PD-L1 was used beyond initial radiological progression while continuing treatment with the same PD-1/PD-L1 agent used before PD, it is considered to be the same regimen. Cohort B1 and B2 participants progressed on or after one platinum-based chemotherapy which was given as part of the anti-PD-1/PD-L1 containing regimen, or was given as a separate regimen, or declined or could not tolerate platinum-based chemotherapy. Participants received no more than one previous chemotherapy regimen unless the prior anti-PD-1/PD-L1 containing regimen to treat stage IV NSCLC did not include platinum-based chemotherapy. In any event, participants of Cohorts B1 and B2 received no more than 2 prior chemotherapy treatments.

Participants of Cohorts A1, A2, B1, and C₁ will receive the IL-2 conjugate (24 μg/kg dose) and pembrolizumab (200 mg) by intravenous infusion once every 3 weeks. Participants of Cohort A3 will receive the IL-2 conjugate (24 μg/kg dose) and pembrolizumab (200 mg) by intravenous infusion once every 3 weeks and will also receive pemetrexed (administered on Day 1 of each 21-day cycle as a 500 mg/m² IV infusion) and one of carboplatin or cisplatin. Participants of Cohort B2 will receive the IL-2 conjugate (24 μg/kg dose), pembrolizumab (200 mg) by intravenous infusion once every 3 weeks and will also receive nab-paclitaxel (administered as a 100 mg/m² IV infusion on Days 1 and 8 of each cycle for 6 cycles). The supervising physician may decide to administer acetaminophen, diphenhydramine, folic acid, vitamin B12, and/or ondansetron if determined to be medically necessary or suitable. Participants of all Cohorts will receive the IL-2 conjugate treatment until disease progression, unacceptable toxicity, or completion of 35 cycles (1 cycle is 21 days).

Key Inclusion Criteria. All participants are males or females 18 years of age or older. Participants in Cohorts A1, A2, A3, B1, and B2 have at least one measurable lesion per RECIST v 1.1. Participants in Cohort C1 have at least one measurable lesion per modified RECIST. All participants have adequate cardiovascular, hematological, liver, renal function, and laboratory parameters. Participants in Cohorts A1, A2, B1, and B2 have histologically or cytologically confirmed diagnosis of Stage IV NSCLC. Participants in A3 have histologically or cytologically confirmed diagnosis of Stage IV NSCLC. Participants in Cohort C1 have histologically confirmed unresectable malignant pleural mesothelioma (MPM). Participants in Cohort A1 have PD-L1 expression TPS ≥50%. Participants in Cohort A2 have PD-L1 expression TPS 1%-49%. Females are eligible to participate if they are not pregnant or breastfeeding, not a woman of childbearing potential (WOCBP) or are a WOCBP that agrees: to use approved contraception method and submit to regular pregnancy testing prior to treatment and for at least 420 days (Cohort A3) or 150 days (Cohorts A1, A2, B1, B2, C₁) after discontinuing study treatment to refrain from donating or cryopreserving eggs for 150 days after discontinuing study treatment. Males are eligible to participate if they agree to refrain from donating or cryopreserving sperm, and either abstain from heterosexual intercourse OR use approved contraception during study treatment and for at least 330 days (Cohort A3) or 210 days (Cohorts A1, A2, B1, B2, C₁) and after discontinuing study treatment. Participants are capable of giving signed informed consent.

Key Exclusion Criteria. All participants have an ECOG performance status of less than 2. Participants do not have a history of allogenic tissue/solid organ transplant. Participants do not have immune-mediated/related toxicity from prior immuno-oncology therapy of Grade 4 or leading to discontinuation. Participants do not have ongoing AEs caused by any prior anti-cancer therapy ≥Grade 2. Participants do not have baseline oxygen saturation (SpO2)≤92% (without oxygen therapy). Participants have not received prior IL-2-based anticancer treatment. Participants are able to temporarily (for at least 36 hours) withhold antihypertensive medications prior to each IL-2 conjugate dosing. Participants have not received a live-virus or live-attenuated vaccination within 28 days of starting treatment with the IL-2 conjugate. Participants do not have any medical or clinical condition, laboratory abnormality, or any specific situation as judged by the supervising physician that would preclude protocol therapy or would make the subject inappropriate for the study. Participants of Cohorts A1, A2, A3, B1, and B2 do not have known driver alterations, such as epidermal growth factor receptor (EGFR), anaplastic lymphoma kinase (ALK), proto-oncogene tyrosine-protein kinase (ROS)1, or BRAF mutation. for participants with non squamous NSCLC. Participants of Cohort A3 do not have uncontrolled pleural/peritoneal effusion, pericardial effusion or ascites requiring recurrent drainage procedures; predominantly squamous cell histology NSCLC; inability to interrupt aspirin or other nonsteroidal anti-inflammatory drugs (NSAIDs), other than an aspirin dose ≤1.3 g per day, for a 5-day period. Participants of Cohorts A1, A2, A3, and C1 have not received prior treatment with an agent that blocks the PD-1/PD-L1 pathway.

The progression of disease can be monitored in patients according to various criteria. The objective response rate (ORR) can be evaluated in patients up to approximately 6 months following administration of the first dose of the IL-2 conjugate and pembrolizumab combination treatment per RECIST 1.1 for Cohort A1-A3 and B1-B2 participants and, per modified RECIST for Cohort C1 participants, up to approximately 6 months following administration of the first dose of the IL-2 conjugate and pembrolizumab combination treatment.

The type, frequency, severity, seriousness, and relationship to study therapy of any adverse event (AE) or laboratory tests or other investigations; drug discontinuation due to AEs can also be evaluated from first administration of the IL-2 conjugate to 30 days after last dose.

Time to response (TTR), defined as the time from the first administration of the IL-2 conjugate to the first tumor assessment at which the overall response was recorded as partial response (PR) or complete response (CR) that is subsequently confirmed and determined per RECIST 1.1 (for NSCLC) or mRECIST (for mesothelioma), can be evaluated.

Duration of response (DoR), defined as the time from first tumor assessment at which the overall response was recorded as partial response (PR) or complete response (CR) that is subsequently confirmed until progressive disease (PD) determined per RECIST 1.1 (for NSCLC) or mRECIST (for mesothelioma) or death from any cause, whichever occurs first, can be evaluated.

Clinical benefit rate (CBR) including CR or PR at any time plus stable disease (SD) of at least 6 months from the first administration of the IL-2 conjugate until PD (per RECIST 1.1 [for NSCLC] or mRECIST [for mesothelioma]), or death from any cause, whichever occurs first, can be evaluated.

Progression free survival (PFS), defined as the time from the date of first the IL-2 conjugate administration to the date of the first documented disease progression as per RECIST 1.1 for NSCLC) or mRECIST (for mesothelioma) or death due to any cause, whichever occurs first, can be evaluated.

Pharmacokinetic parameters, such as concentration of IL-2 conjugate, and the incidence of anti-drug antibodies (ADAs) against the IL-2 conjugate, can also be evaluated in patients at various time points throughout the study.

In some embodiments, the individual receiving treatment (i.e., a participant in Cohort A1, A2, A3, B1, B2, or C₁) shows a decrease in the size of target lesions in response to treatment. In some embodiments, the individual shows a complete response (CR) following treatment. In some embodiments, the individual shows a partial response (PR) following treatment. In some embodiments, the individual shows stable disease (SD) following treatment. In some embodiments, the individual shows PR, CR, or SD after the first tumor assessment (such as after 3 cycles). In some embodiments, the individual shows PR, CR, or SD after the second tumor assessment (such as after 4 or more cycles). In some embodiments, the individual shows PR, CR, or SD after the third, fourth, or subsequent tumor assessment.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby. The disclosures of all patent and scientific literature cited herein are expressly incorporated herein in their entirety by reference.

What is claimed is:

1. A method of treating lung cancer in a subject in need thereof, comprising administering to the subject (a) an IL-2 conjugate, and (b) an anti-PD-1 antibody or antigen-binding fragment thereof, wherein:
the IL-2 conjugate comprises the amino acid sequence of SEQ ID NO: 1 wherein the amino acid at position P64 is replaced by the structure of Formula (I): 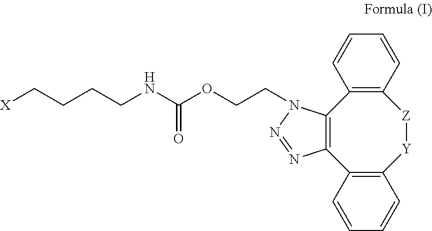
wherein:
Z is CH₂ and Y is 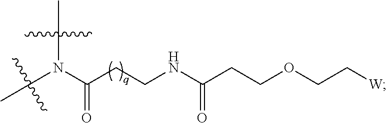
Y is CH₂ and Z is 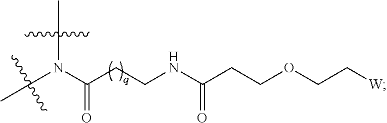
Z is CH₂ and Y is; 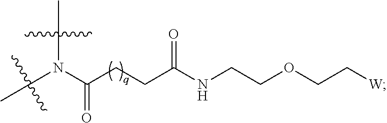
or
Y is CH₂ and Z is 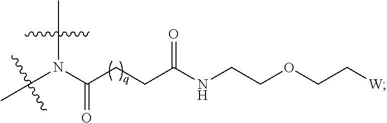
W is a PEG group having an average molecular weight of about 25 kDa-35 kDa;
q is 1, 2, or 3;
X is an L-amino acid having the structure: 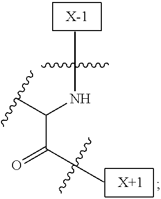 [SMILES: "X-1" ; *NC(*)C(*)=O ; "X+1"]
X−1 indicates the point of attachment to the preceding amino acid residue; and
X+1 indicates the point of attachment to the following amino acid residue; and
wherein the anti-PD-1 antibody or antigen-binding fragment thereof comprises light chain complementarity determining regions (CDRs) comprising a sequence of amino acids as set forth in SEQ ID NOs: 11, 12 and 13 and heavy chain CDRs comprising a sequence of amino acids as set forth in SEQ ID NOs: 16, 17 and 18.

2. A method of treating lung cancer in a subject in need thereof, comprising administering to the subject (a) an IL-2 conjugate, and (b) an anti-PD-1 antibody or antigen-binding fragment thereof, wherein:
the lung cancer is non-squamous non-small cell lung cancer (NSCLC), pleural mesothelioma, unresectable lung cancer, stage IV lung cancer, NSCLC having a PD-L1 tumor proportion score greater than or equal to 50%, or NSCLC having a PD-L1 tumor progression score of less than 50% or of 1-49%; and
the IL-2 conjugate comprises the amino acid sequence of SEQ ID NO: 1 wherein the amino acid at position P64 is replaced by the structure of Formula (I): 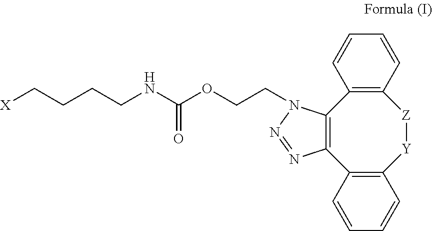
wherein:
Z is CH₂ and Y is 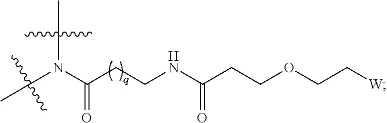
Y is CH₂ and Z is 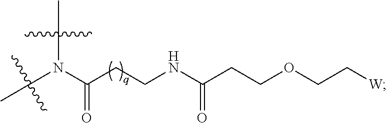
Z is CH₂ and Y is; 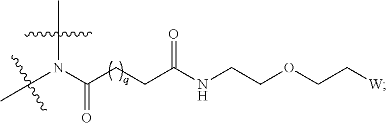
or
Y is CH₂ and Z is 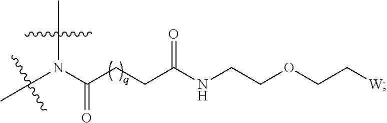
W is a PEG group having an average molecular weight of about 25 kDa-35 kDa;
q is 1, 2, or 3;
X is an L-amino acid having the structure: 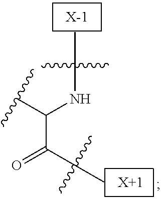 [SMILES: "X-1" ; *NC(*)C(*)=O ; "X+1"]
X−1 indicates the point of attachment to the preceding amino acid residue; and
X+1 indicates the point of attachment to the following amino acid residue,
wherein the anti-PD-1 antibody or antigen-binding fragment thereof comprises light chain complementarity determining regions (CDRs) comprising a sequence of amino acids as set forth in SEQ ID NOs: 11, 12 and 13 and heavy chain CDRs comprising a sequence of amino acids as set forth in SEQ ID NOs: 16, 17 and 18.

3. A method of treating lung cancer in a subject in need thereof, comprising:
selecting a subject having lung cancer, wherein the subject is selected on the basis of one or more attributes comprising (i) the lung cancer being non-squamous non-small cell lung cancer (NSCLC); (ii) the lung cancer being pleural mesothelioma; (iii) the lung cancer being unresectable lung cancer; (iv) the lung cancer being stage IV lung cancer; (v) the lung cancer being NSCLC having a PD-L1 tumor proportion score greater than or equal to 50%; (vi) the lung cancer being NSCLC having a PD-L1 tumor progression score of less than 50% or of 1-49%; and
administering to the subject (a) an IL-2 conjugate, and (b) an anti-PD-1 antibody or antigen-binding fragment thereof, wherein:
the IL-2 conjugate comprises the amino acid sequence of SEQ ID NO: 1 wherein the amino acid at position P64 is replaced by the structure of Formula (I): 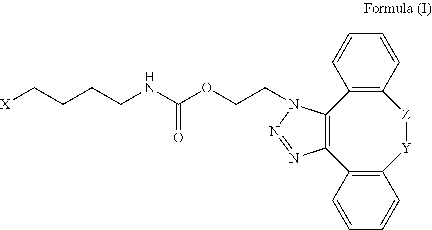
wherein:
Z is CH₂ and Y is 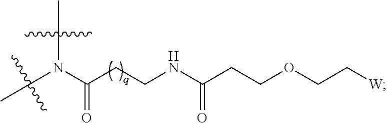
Y is CH₂ and Z is 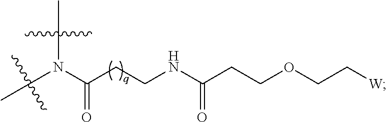
Z is CH₂ and Y is 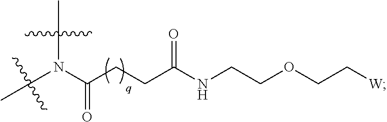
or
Y is CH₂ and Z is 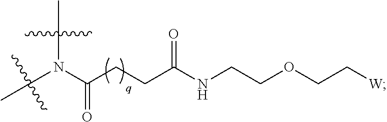
W is a PEG group having an average molecular weight of about 25 kDa-35 kDa;
q is 1, 2, or 3;
X is an L-amino acid having the structure: 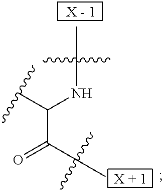 [SMILES: "X - 1" ; *NC(*)C(*)=O ; "X + 1"]
X−1 indicates the point of attachment to the preceding amino acid residue; and
X+1 indicates the point of attachment to the following amino acid residue,
wherein the anti-PD-1 antibody or antigen-binding fragment thereof comprises light chain complementarity determining regions (CDRs) comprising a sequence of amino acids as set forth in SEQ ID NOs: 11, 12 and 13 and heavy chain CDRs comprising a sequence of amino acids as set forth in SEQ ID NOs: 16, 17 and 18.

4. The method of any one of claims 1-3, further comprising administering cisplatin to the subject.

5. A method of treating lung cancer in a subject in need thereof, comprising administering to the subject (a) an IL-2 conjugate, (b) an anti-PD-1 antibody or antigen-binding fragment thereof, and (c) cisplatin, wherein:
the IL-2 conjugate comprises the amino acid sequence of SEQ ID NO: 1 wherein the amino acid at position P64 is replaced by the structure of Formula (I): 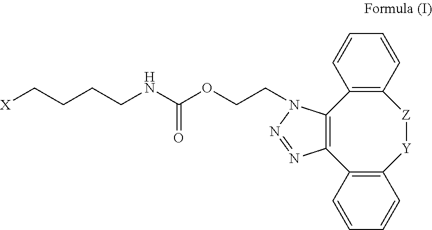
wherein:
Z is CH₂ and Y is 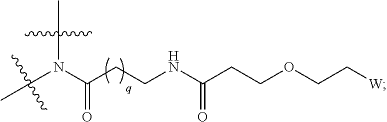
Y is CH₂ and Z is 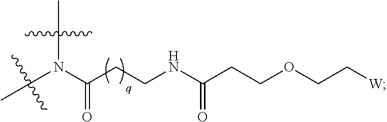
Z is CH₂ and Y is 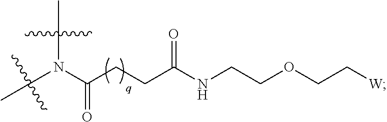
or
Y is CH₂ and Z is 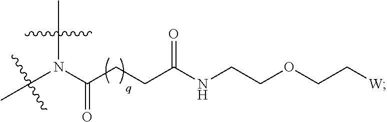
W is a PEG group having an average molecular weight of about 25 kDa-35 kDa;
q is 1, 2, or 3;
X is an L-amino acid having the structure: 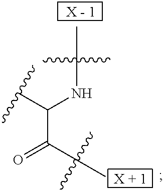 [SMILES: "X - 1" ; *NC(*)C(*)=O ; "X + 1"]
X−1 indicates the point of attachment to the preceding amino acid residue; and
X+1 indicates the point of attachment to the following amino acid residue,
wherein the anti-PD-1 antibody or antigen-binding fragment thereof comprises light chain complementarity determining regions (CDRs) comprising a sequence of amino acids as set forth in SEQ ID NOs: 11, 12 and 13 and heavy chain CDRs comprising a sequence of amino acids as set forth in SEQ ID NOs: 16, 17 and 18.

6. The method of any one of claims 1-5, wherein the lung cancer is NSCLC.

7. The method of any one of claims 1-6, wherein the lung cancer is unresectable.

8. The method of any one of claims 1-7, wherein the lung cancer is stage IV.

9. The method of any one of claims 1-8, wherein the lung cancer is non-squamous NSCLC.

10. The method of any one of claims 1-9, wherein the lung cancer is pleural mesothelioma.

11. The method of any one of claims 1-10, comprising administering to the subject about 8 μg/kg of the IL-2 conjugate.

12. The method of any one of claims 1-10, comprising administering to the subject about 16 μg/kg of the IL-2 conjugate.

13. The method of any one of claims 1-10, comprising administering to the subject about 24 μg/kg of the IL-2 conjugate.

14. The method of any one of claims 1-10, comprising administering to the subject about 32 μg/kg of the IL-2 conjugate.

15. The method of any one of claims 1-14, further comprising administering pemetrexed to the subject.

16. The method of any one of claims 1-15, further comprising administering carboplatin to the subject.

17. The method of any one of claims 1-16, further comprising administering nab-paclitaxel to the subject.

18. The method of any one of claims 1-17, wherein in the IL-2 conjugate the PEG group has an average molecular weight of about 30 kDa.

19. The method of any one of claims 1-18, wherein in the IL-2 conjugate Z is CH₂ and Y is

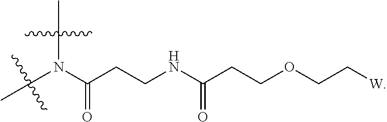

20. The method of any one of claims 1-18, wherein in the IL-2 conjugate Y is CH₂ and Z is

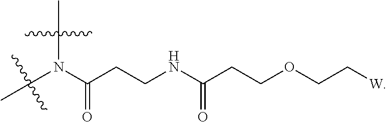

21. The method of any one of claims 1-18, wherein in the IL-2 conjugate Z is CH₂ and Y is

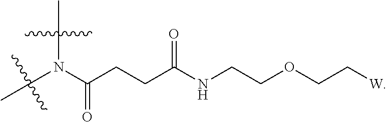

22. The method of any one of claims 1-18, wherein in the IL-2 conjugate Y is CH₂ and Z is

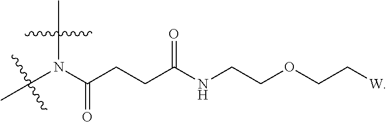

23. The method of any one of claims 1-18, wherein the structure of Formula (I) has the structure of Formula (IV) or Formula (V), or is a mixture of Formula (IV) and Formula (V): 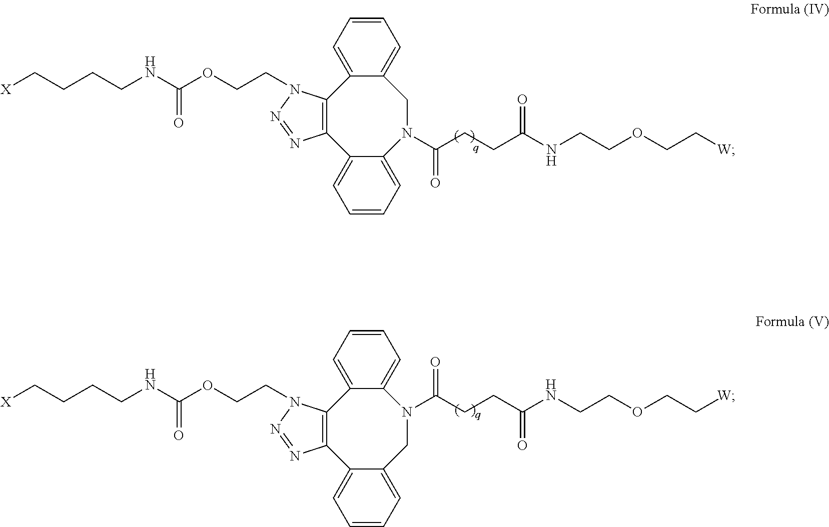
wherein:
q is 1, 2, or 3;
X is an L-amino acid having the structure: 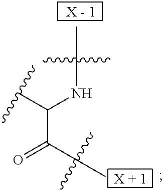 [SMILES: "X - 1" ; *NC(*)C(*)=O ; "X + 1"]
X−1 indicates the point of attachment to the preceding amino acid residue; and
X+1 indicates the point of attachment to the following amino acid residue.

24. The method of any one of claims 1-18, wherein the structure of Formula (I) has the structure of Formula (XII) or Formula (XIII), or is a mixture of Formula (XII) and Formula (XIII): 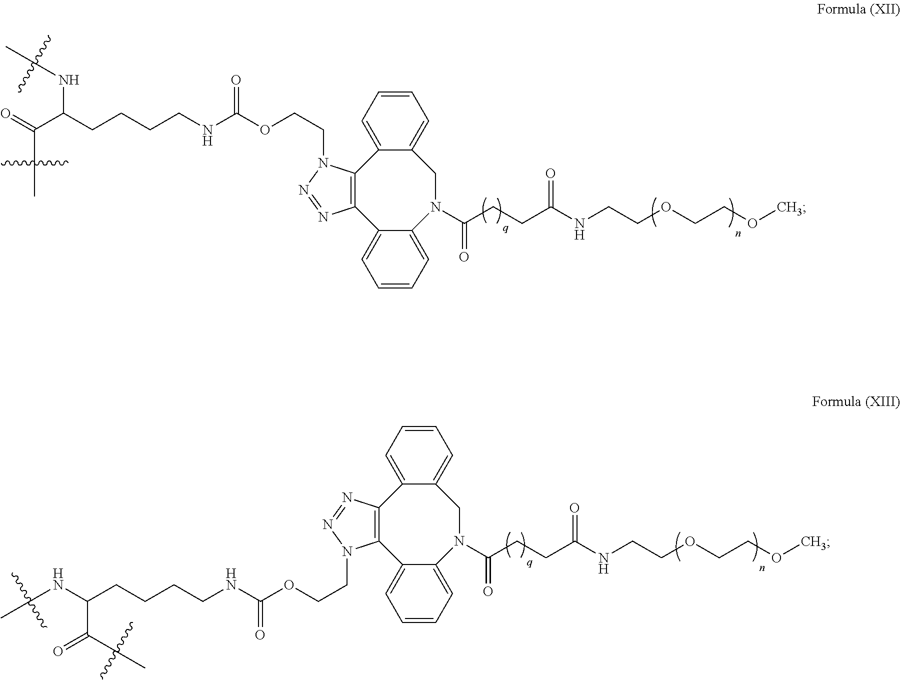
wherein:
n is an integer such that —(OCH₂CH₂)ₙ—OCH₃ has a molecular weight of about 30 kDa;
q is 1, 2, or 3; and
the wavy lines indicate covalent bonds to amino acid residues within SEQ ID NO: 1 that are not replaced.

25. The method of any one of claims 1-24, wherein q is 1.

26. The method of any one of claims 1-24, wherein q is 2.

27. The method of any one of claims 1-24, wherein q is 3.

28. The method of any one of claims 1-27, wherein the IL-2 conjugate is administered to the subject about once every two weeks, about once every three weeks, or about once every 4 weeks.

29. The method of any one of claims 1-28, wherein the IL-2 conjugate and the anti-PD-1 antibody or antigen-binding fragment thereof are administered to the subject about once every two weeks, about once every three weeks, or about once every 4 weeks.

30. The method of any one of claims 1-29, wherein the IL-2 conjugate is a pharmaceutically acceptable salt, solvate, or hydrate.

31. The method of any one of claims 1-30, wherein the method comprises administering:
(iii) about 200 mg of an anti-PD-1 antibody, or antigen binding fragment thereof to the patient every approximately three weeks; or
(iv) about 400 mg of an anti-PD-1 antibody, or antigen binding fragment thereof, to the patient every approximately six weeks.

32. The method of any one of claims 1-31, wherein the IL-2 conjugate and the anti-PD-1 antibody or antigen-binding fragment thereof are administered separately.

33. The method of claim 32, wherein the IL-2 conjugate and the anti-PD-1 antibody or antigen-binding fragment thereof are administered sequentially.

34. The method of claim 32 or 33, wherein the IL-2 conjugate is administered before the anti-PD-1 antibody or antigen-binding fragment thereof.

35. The method of claim 32 or 33, wherein the IL-2 conjugate is administered after the anti-PD-1 antibody or antigen-binding fragment thereof.

36. The method of any one of claims 1-35, wherein the IL-2 conjugate is administered to the subject by subcutaneous administration.

37. The method of any one of claims 1-36, wherein the IL-2 conjugate and the anti-PD-1 antibody or antigen-binding fragment thereof are administered to the subject by subcutaneous administration.

38. The method of any one of claims 1-35, wherein the IL-2 conjugate is administered to the subject by intravenous administration.

39. The method of any one of claims 1-35, wherein the IL-2 conjugate and the anti-PD-1 antibody or antigen-binding fragment thereof are administered to the subject by intravenous administration.

40. The method of any one of claims 1-39, further comprising administering acetaminophen to the subject.

41. The method of any one of claims 1-40, further comprising administering diphenhydramine to the subject.

42. The method of claim 40 or 41, wherein the acetaminophen and/or diphenhydramine is administered to the subject before administering the IL-2 conjugate.

43. The method of any one of claims 1-42, further comprising selecting the subject to whom the IL-2 conjugate and the anti-PD-1 antibody or antigen-binding fragment thereof are administered at least in part on the basis of the lung cancer being non-squamous NSCLC.

44. The method of any one of claims 1-43, further comprising selecting the subject to whom the IL-2 conjugate and the anti-PD-1 antibody or antigen-binding fragment thereof are administered at least in part on the basis of the lung cancer being pleural mesothelioma.

45. The method of any one of claims 1-44, further comprising selecting the subject to whom the IL-2 conjugate and the anti-PD-1 antibody or antigen-binding fragment thereof are administered at least in part on the basis of the lung cancer being unresectable lung cancer.

46. The method of any one of claims 1-45, further comprising selecting the subject to whom the IL-2 conjugate and the anti-PD-1 antibody or antigen-binding fragment thereof are administered at least in part on the basis of the lung cancer being stage IV lung cancer.

47. The method of any one of claims 1-46, further comprising selecting the subject to whom the IL-2 conjugate and the anti-PD-1 antibody or antigen-binding fragment thereof are administered at least in part on the basis of the lung cancer being NSCLC having a PD-L1 tumor proportion score greater than or equal to 50%.

48. The method of any one of claims 1-47, further comprising selecting the subject to whom the IL-2 conjugate and the anti-PD-1 antibody or antigen-binding fragment thereof are administered at least in part on the basis of the lung cancer being NSCLC having a PD-L1 tumor progression score of less than 50% or of 1-49%.

49. An IL-2 conjugate for use in the method of any one of claims 1-48.

50. Use of an IL-2 conjugate for the manufacture of a medicament for the method of any one of claims 1-49.

51. The method, IL-2 conjugate for use, or use of any of the preceding claims, wherein the anti-PD-1 antibody or antigen-binding fragment thereof comprises:
(a) a heavy chain variable region comprising a sequence of amino acids as set forth in SEQ ID NO:19, or a variant of SEQ ID NO:19, and
(b) a light chain variable region comprising a sequence of amino acids as set forth in SEQ ID NO:14, or a variant of SEQ ID NO:14.

52. The method, IL-2 conjugate for use, or use of any of the preceding claims, wherein the anti-PD-1 antibody or antigen-binding fragment thereof is a monoclonal antibody comprising a heavy chain comprising a sequence of amino acids as set forth in SEQ ID NO:20 and a light chain comprising a sequence of amino acids as set forth in SEQ ID NO:15.


SEQUENCE LISTING (20 sequences)

SEQ ID NO: 1 - protein, 132 aa, synthetic construct
  REGION 1..132: note = IL-2 conjugate
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 2 - protein, 132 aa, synthetic construct
  REGION 1..132: note = IL-2 conjugate
  SITE 64: note = misc_feature - N6-((2-azidoethoxy)-carbonyl)-L-lysine stably-conjugated to PEG via DBCO-mediated click chemistry
PTSSSTKKTQLQLEHLLLDLQMILNGINNYKNPKLTRMLTFKFYMPKKATELKHLQCLEEELKXLEEVLNLAQSKNFHLRPRDLISNINVIVLELKGSETTFMCEYADETATIVEFLNRWITFSQSIISTLT

SEQ ID NO: 3 - protein, 133 aa, synthetic construct
  REGION 1..133: note = IL-2 conjugate
  SITE 65: note = misc_feature - N6-((2-azidoethoxy)-carbonyl)-L-lysine stably-conjugated to PEG via DBCO-mediated click chemistry
APTSSSTKKTQLQLEHLLLDLQMILNGINNYKNPKLTRMLTFKFYMPKKATELKHLQCLEEELKXLEEVLNLAQSKNFHLRPRDLISNINVIVLELKGSETTFMCEYADETATIVEFLNRWITFSQSIISTLT

SEQ ID NO: 4 - protein, 133 aa, synthetic construct
  REGION 1..133: note = IL-2 conjugate
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 5-10 - skipped (6 entries)

SEQ ID NO: 11 - protein, 15 aa, synthetic construct
  REGION 1..15: note = hPD-1.09A light chain CDR1
RASKGVSTSGYSYLH

SEQ ID NO: 12 - protein, 7 aa, synthetic construct
  REGION 1..7: note = hPD-1.09A light chain CDR2
LASYLES

SEQ ID NO: 13 - protein, 9 aa, synthetic construct
  REGION 1..9: note = hPD-1.09A light chain CDR3
QHSRDLPLT

SEQ ID NO: 14 - protein, 111 aa, synthetic construct [sequence omitted]

SEQ ID NO: 15 - protein, 218 aa, synthetic construct
  REGION 1..218: note = light chain of pembrolizumab
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 5 aa, synthetic construct
  REGION 1..5: note = hPD-1.09A heavy chain CDR1
NYYMY

SEQ ID NO: 17 - protein, 17 aa, synthetic construct
  REGION 1..17: note = hPD-1.09A heavy chain CDR2
GINPSNGGTNFNEKFKN

SEQ ID NO: 18 - protein, 11 aa, synthetic construct
  REGION 1..11: note = hPD-1.09A heavy chain CDR3
RDYRFDMGFDY

SEQ ID NO: 19 - protein, 120 aa, synthetic construct [sequence omitted]

SEQ ID NO: 20 - protein, 447 aa, synthetic construct
  REGION 1..447: note = heavy chain of pembrolizumab
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK